SEQUENCE LISTING (88 sequences)

SEQ ID NO: 1 - DNA, 1961 bp, Artificial
  antisense DNA sequence of LEREPO4
ttttttttttttttttttggattttaatgtatgatggtctttatttacaactttattggcaaaaaagaaaagggaaacttttaaacaatttaacacagggcattgtagctgatcctgtcagataaaagaagttccattttaaatgtccatctaattgtccaaagatacacaatactgaatctgcatacgcagtttcctttatgaagtacagtgctcatgttttaggcagtcttctaaacatacaaatacagaggaaattaaatcactcattaaaaactgtgtcaaatgaagggtattttaaatataagtttgttgtttctggtaatagcacatgcccaaatgaaaaccaaaagatggaaaggcagatacacttcttacttgggtggacagttacaacatcaatcattttctgcaatgaccattatattttcaatttatcatgaactactctgaacttactatgagatgtagccaaagtcagaagagaagatgtagggacaagatgccttttttgcaggaggttaactcctccagaatcagcataggaaataaacatcctgttgattctaggtggaaagaaattaatgtagtcaatttcaactcgtgctgagctgacttaattttttcagcgatgtgtttggtgtcattcttctaaatcaagtgtatttaattcttcttctagttcatccaaatcctctccagtgaaaagattttcatcaacaggaacagcatcaatggctccattttccgttccctccctctcgttgtcctcttccaagtcacttctttcaccattttcagccctacctccagaagcttcacttaatttgttttcatctttatctgaagtatatgtgctgaatctttcaagactggctacagtaatacctgtttcatctacatctcttgggatgtacaggcttaaatctatgtcatttacactcactgaatcatcaacctcatcaccacctgttccctgggtgtagcgggtatcatctgcttcctcatcatcatcattgaccagttcaggacgaaattcaaacacttcacgaccactgatcactagtgctttccctgctttgaagtcagctttccttctttccatatcttgttcaagtttatcaatcttttcttgtcttttccttttcttccaggcaagaaaagattctagagtgattttggtaacatttggacctagggcagaacgctctctctcaattagatcttctaatgaaatttcatcttctttctcttctttctttttatcttttttcaacacaaatccaggaggaagtgcatgacgatacatgcaaatatcaccccctccagggcatacccaaaaccagccatacttgttgttttcaatagcttccaggaaatgcttgcacactatttgagtttttggttttttcttttccgcctcaccgtgcttcttgttcactacttcttccagctttttctcatcccaattatccatagtatctttttcaagttcttcatctcttgcatcaatgtaaacacttcgcttttcacattttctctccagagtcaagtcatgggagaacttacacttatctcctttagtacactgtccttgcttgaagaatgcacatactacagacttgggatctgcacctttacttattttttgagcagcaactacaggtttgaacagctcatttagctcctgcaattctttcttcttgtcatccttcttcaatttcttttcagcttcactctgtgctacctgacgtggattttgttgaccaaatttaacttgatgtgtgacagccttgataaacttctgttgctttgctcctttcttattcttcaaaccgaaagttttgtcttcgataatcttctccttctttttttgctccgcctttttgctgcccccggcctgagcctgtttcttggggggcattgcggagacagatggcaccggccagacttacgcgcag

SEQ ID NO: 2 - DNA, 1061 bp, Artificial
  antisense DNA sequence of GliPR
gtaagagtattgctataacatatatttttttctgaaatgttttaaaatagaagtacacctaattgttattggttaaaaaaaagccatatgtgaatgaggttggtttttgggttttttctttcctgaattgtattagtccaaaagaactaaattagggtacttgagctgtaccaaaatggtaattataacagacagtattagaattactgaattaacaatgagaaagagagaagtgtatctgttacgtggatatatgggccagcctggatatacaacagagtagtaacgtttcacttggtctcgctgtcggttaacacagagattgtccaaacacttgtcattattggggcaggcactgcaggtggctcctctcttatatggccaagttgggtaattccctcctggtccgtagttgcatataaaatgtgctccattggaaagagcgtcaaagccagaaactttagggcaaaattgaactgcgcagccaactttgtaactatctgcccaaacaacctgagtgtagtggccacagacttttttgcatatccgagtcttgaagtcatagtcctggatttcgtcataccagtttgtgatggcggaagacacagaaaaaatgggcacagacccagtccagatgttctctcccagtgaagtgaagtttgggtgcagcttgtggggtggcttcagccgtgtattatgtgaaaactggcaattgctggcccatgcttttgcaatttgggctagtgctgggtcccaagtcatgtatagcatatcactggctgttggtttcacctctgatcggaacttgttatggattcgaacgcagtctttgatgaaatcttcattttcgatatctggcaaaatatttgctgtgtgtgaataattggagacaaaagaaaccatccaggctattgtagcaagtgtgacacgcatgctttgtctggcatggagccttgggaggctgcagtccagagccgccgcagtttctgagagtgtgattgcctgagtgctcagcccacagctttgcaaacctagccgacttcagctttgagaaaacagag

SEQ ID NO: 3 - DNA, 1961 bp, Homo sapiens
ctgcgcgtaagtctggccggtgccatctgtctccgcaatgccccccaagaaacaggctcaggccgggggcagcaaaaaggcggagcaaaaaaagaaggagaagattatcgaagacaaaactttcggtttgaagaataagaaaggagcaaagcaacagaagtttatcaaggctgtcacacatcaagttaaatttggtcaacaaaatccacgtcaggtagcacagagtgaagctgaaaagaaattgaagaaggatgacaagaagaaagaattgcaggagctaaatgagctgttcaaacctgtagttgctgctcaaaaaataagtaaaggtgcagatcccaagtctgtagtatgtgcattcttcaagcaaggacagtgtactaaaggagataagtgtaagttctcccatgacttgactctggagagaaaatgtgaaaagcgaagtgtttacattgatgcaagagatgaagaacttgaaaaagatactatggataattgggatgagaaaaagctggaagaagtagtgaacaagaagcacggtgaggcggaaaagaaaaaaccaaaaactcaaatagtgtgcaagcatttcctggaagctattgaaaacaacaagtatggctggttttgggtatgccctggagggggtgatatttgcatgtatcgtcatgcacttcctcctggatttgtgttgaaaaaagataaaaagaaagaagagaaagaagatgaaatttcattagaagatctaattgagagagagcgttctgccctaggtccaaatgttaccaaaatcactctagaatcttttcttgcctggaagaaaaggaaaagacaagaaaagattgataaacttgaacaagatatggaaagaaggaaagctgacttcaaagcagggaaagcactagtgatcagtggtcgtgaagtgtttgaatttcgtcctgaactggtcaatgatgatgatgaggaagcagatgatacccgctacacccagggaacaggtggtgatgaggttgatgattcagtgagtgtaaatgacatagatttaagcctgtacatcccaagagatgtagatgaaacaggtattactgtagccagtcttgaaagattcagcacatatacttcagataaagatgaaaacaaattaagtgaagcttctggaggtagggctgaaaatggtgaaagaagtgacttggaagaggacaacgagagggagggaacggaaaatggagccattgatgctgttcctgttgatgaaaatcttttcactggagaggatttggatgaactagaagaagaattaaatacacttgatttagaagaatgacaccaaacacatcgctgaaaaaattaagtcagctcagcacgagttgaaattgactacattaatttctttccacctagaatcaacaggatgtttatttcctatgctgattctggaggagttaacctcctgcaaaaaaggcatcttgtccctacatcttctcttctgactttggctacatctcatagtaagttcagagtagttcatgataaattgaaaatataatggtcattgcagaaaatgattgatgttgtaactgtccacccaagtaagaagtgtatctgcctttccatcttttggttttcatttgggcatgtgctattaccagaaacaacaaacttatatttaaaatacccttcatttgacacagtttttaatgagtgatttaatttcctctgtatttgtatgtttagaagactgcctaaaacatgagcactgtacttcataaaggaaactgcgtatgcagattcagtattgtgtatctttggacaattagatggacatttaaaatggaacttcttttatctgacaggatcagctacaatgccctgtgttaaattgtttaaaagtttcccttttcttttttgccaataaagttgtaaataaagaccatcatacattaaaatccaaaaaaaaaaaaaaaaaa

SEQ ID NO: 4 - protein, 426 aa, Homo sapiens
MPPKKQAQAGGSKKAEQKKKEKIIEDKTFGLKNKKGAKQQKFIKAVTHQVKFGQQNPRQVAQSEAEKKLKKDDKKKELQELNELFKPVVAAQKISKGADPKSVVCAFFKQGQCTKGDKCKFSHDLTLERKCEKRSVYIDARDEELEKDTMDNWDEKKLEEVVNKKHGEAEKKKPKTQIVCKHFLEAIENNKYGWFWVCPGGGDICMYRHALPPGFVLKKDKKKEEKEDEISLEDLIERERSALGPNVTKITLESFLAWKKRKRQEKIDKLEQDMERRKADFKAGKALVISGREVFEFRPELVNDDDEEADDTRYTQGTGGDEVDDSVSVNDIDLSLYIPRDVDETGITVASLERFSTYTSDKDENKLSEASGGRAENGERSDLEEDNEREGTENGAIDAVPVDENLFTGEDLDELEEELNTLDLEE

SEQ ID NO: 5 - DNA, 1061 bp, Homo sapiens
ctctgttttctcaaagctgaagtcggctaggtttgcaaagctgtgggctgagcactcaggcaatcacactctcagaaactgcggcggctctggactgcagcctcccaaggctccatgccagacaaagcatgcgtgtcacacttgctacaatagcctggatggtttcttttgtctccaattattcacacacagcaaatattttgccagatatcgaaaatgaagatttcatcaaagactgcgttcgaatccataacaagttccgatcagaggtgaaaccaacagccagtgatatgctatacatgacttgggacccagcactagcccaaattgcaaaagcatgggccagcaattgccagttttcacataatacacggctgaagccaccccacaagctgcacccaaacttcacttcactgggagagaacatctggactgggtctgtgcccattttttctgtgtcttccgccatcacaaactggtatgacgaaatccaggactatgacttcaagactcggatatgcaaaaaagtctgtggccactacactcaggttgtttgggcagatagttacaaagttggctgcgcagttcaattttgccctaaagtttctggctttgacgctctttccaatggagcacattttatatgcaactacggaccaggagggaattacccaacttggccatataagagaggagccacctgcagtgcctgccccaataatgacaagtgtttggacaatctctgtgttaaccgacagcgagaccaagtgaaacgttactactctgttgtatatccaggctggcccatatatccacgtaacagatacacttctctctttctcattgttaattcagtaattctaatactgtctgttataattaccattttggtacagctcaagtaccctaatttagttcttttggactaatacaattcaggaaagaaaaaacccaaaaaccaacctcattcacatatggcttttttttaaccaataacaattaggtgtacttctattttaaaacatttcagaaaaaaatatatgttatagcaatactcttac

SEQ ID NO: 6 - protein, 266 aa, Homo sapiens
MRVTLATIAWMVSFVSNYSHTANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRYTSLFLIVNSVILILSVIITILVQLKYPNLVLLD

SEQ ID NO: 7 - protein, 400 aa, Artificial
  trans-dominant negative mutant of LEREPO4 lacking amino acids 100 to 125
MPPKKQAQAGGSKKAEQKKKEKIIEDKTFGLKNKKGAKQQKFIKAVTHQVKFGQQNPRQVAQSEAEKKLKKDDKKKELQELNELFKPVVAAQKISKGADTLERKCEKRSVYIDARDEELEKDTMDNWDEKKLEEVVNKKHGEAEKKKPKTQIVCKHFLEAIENNKYGWFWVCPGGGDICMYRHALPPGFVLKKDKKKEEKEDEISLEDLIERERSALGPNVTKITLESFLAWKKRKRQEKIDKLEQDMERRKADFKAGKALVISGREVFEFRPELVNDDDEEADDTRYTQGTGGDEVDDSVSVNDIDLSLYIPRDVDETGITVASLERFSTYTSDKDENKLSEASGGRAENGERSDLEEDNEREGTENGAIDAVPVDENLFTGEDLDELEEELNTLDLEE

SEQ ID NO: 8 - protein, 11 aa, Artificial
  CRISP family signature 1 in GliPR
GHYTQVVWADS

SEQ ID NO: 9 - protein, 12 aa, Artificial
  CRISP family signature 2 in GliPR
HFICNYGPGGNY

SEQ ID NO: 10 - protein, 29 aa, Artificial
  consensus motif of the CRISP family signature 1
  misc_feature (26)..(26): Xaa can be any naturally occurring amino acid
GDERHRFYWHTVSQALIVMLIVMAWXSTN

SEQ ID NO: 11 - protein, 39 aa, Artificial
  consensus motif of the CRISP family signature 2
  misc_feature (14)..(14): Xaa can be any naturally occurring amino acid
  misc_feature (22)..(22): Xaa can be any naturally occurring amino acid
  misc_feature (27)..(27): Xaa can be any naturally occurring amino acid
LIVMFYHLIVMFYXCNQRHSYXPARHXGLNLIVMFYWDN

SEQ ID NO: 12 - protein, 21 aa, Artificial
  N-terminal signal sequence of GliPR
MRVTLATIAWMVSFVSNYSHT

SEQ ID NO: 13 - protein, 23 aa, Artificial
  putative transmembrane domain of GliPR
YTSLFLIVNSVILILSVIITILV

SEQ ID NO: 14 - DNA, 21 bp, Artificial
  siRNA oligonucleotide si-LEREPO4-1 antisense
  siRNA oligonucleotide si-LEREPO4-1 antisense (1)..(21)
acuucuguugcuuugcucctt

SEQ ID NO: 15 - DNA, 21 bp, Artificial
  siRNA oligonucleotide si-LEREPO4-2 antisense
aacuugaugugugacagcctt

SEQ ID NO: 16 - DNA, 21 bp, Artificial
  siRNA oligonucleotide si-LEREPO4-3 antisense
aucuuucuucuugucaucctt

SEQ ID NO: 17 - DNA, 21 bp, Artificial
  siRNA oligonucleotide si-GliPR-1 antisense
acuggcuguugguuucacctc

SEQ ID NO: 18 - DNA, 21 bp, Artificial
  siRNA oligonucleotide si- GliPR-2 antisense
agucuugaagucauagucctg

SEQ ID NO: 19 - DNA, 21 bp, Artificial
  siRNA oligonucleotide si- GliPR-3 antisense
acuaucugcccaaacaacctg

SEQ ID NO: 20 - DNA, 21 bp, Artificial
  siRNA oligonucleotide si-nons-Rho antisense
acgugacacguucgaagaatt

SEQ ID NO: 21 - DNA, 23 bp, Artificial
  siRNA oligonucleotide HIV-1 p24 antisense
ccugucucucaguacaaucdtdt

SEQ ID NO: 22 - DNA, 23 bp, Artificial
  siRNA oligonucleotide HIV-1 p24 sense
gauuguacugagagacaggdtdt

SEQ ID NO: 23 - DNA, 21 bp, Artificial
  siRNA oligonucleotide si-LEREPO4-1 sense
ggagcaaagcaacagaagutt

SEQ ID NO: 24 - DNA, 21 bp, Artificial
  siRNA oligonucleotide si-LEREPO4-2 sense
ggcugucacacaucaaguutt

SEQ ID NO: 25 - DNA, 21 bp, Artificial
  siRNA oligonucleotide si-LEREPO4-3 sense
ggaugacaagaagaaagautt

SEQ ID NO: 26 - DNA, 21 bp, Artificial
  siRNA oligonucleotide si-GliPR-1 sense
ggugaaaccaacagccagutt

SEQ ID NO: 27 - DNA, 21 bp, Artificial
  siRNA oligonucleotide si-GliPR-2 sense
ggacuaugacuucaagacutt

SEQ ID NO: 28 - DNA, 21 bp, Artificial
  siRNA oligonucleotide si-GliPR-3 sense
gguuguuugggcagauagutt

SEQ ID NO: 29 - DNA, 21 bp, Artificial
  siRNA oligonucleotide si-nons-Rho sense
uucuucgaacgugucacgutt

SEQ ID NO: 30 - DNA, 18 bp, Artificial
  oligonucleotide LEREPO4 forward
ggtgccatctgtctccgc

SEQ ID NO: 31 - DNA, 25 bp, Artificial
  oligonucleotide LEREPO4 backward
tctgttgctttgctcctttcttatt

SEQ ID NO: 32 - DNA, 22 bp, Artificial
  oligonucleotide LEREPO4 TaqMan
tgccccccaagaaacaggctca

SEQ ID NO: 33 - DNA, 19 bp, Artificial
  oligonucleotide GliPR forward
tgccagacaaagcatgcgt

SEQ ID NO: 34 - DNA, 25 bp, Artificial
  oligonucleotide GliPR backward
gctgtgtgtgaataattggagacaa

SEQ ID NO: 35 - DNA, 31 bp, Artificial
  oligonucleotide GliPR TaqMan
tcacacttgctacaatagcctggatggtttc

SEQ ID NO: 36 - DNA, 26 bp, Artificial
  oligonucleotide HIV-pol forward
aatttcaccagtactacggttaaggc

SEQ ID NO: 37 - DNA, 36 bp, Artificial
  oligonucleotide HIV-pol backward
ctttaattctttattcatagattctactactccttg

SEQ ID NO: 38 - DNA, 22 bp, Artificial
  oligonucleotide HIV-pol TaqMan
tgttggtgggcgggaatcaagc

SEQ ID NO: 39 - DNA, 19 bp, Artificial
  oligonucleotide GAPDH forward
gaaggtgaaggtcggagtc

SEQ ID NO: 40 - DNA, 20 bp, Artificial
  oligonucleotide GAPDH backward
gaagatggtgatgggatttc

SEQ ID NO: 41 - DNA, 20 bp, Artificial
  oligonucleotide GAPDH TaqMan
caagcttcccgttctcagcc

SEQ ID NO: 42 - DNA, 31 bp, Artificial
  F-SacII-GliPR-EGFP
ccgcggatgcgtgtcacacttgctacaatag

SEQ ID NO: 43 - DNA, 36 bp, Artificial
  B-BamHI-GliPR-EGFP
ggatccgtccaaaagaactaaattagggtacttgag

SEQ ID NO: 44 - protein, 15 aa, Artificial
  LEREPO4-C-Term
CDELEEELNTLDLEE

SEQ ID NO: 45 - protein, 426 aa, Homo sapiens; identical to SEQ ID NO: 4
  ref <308>: DATABASE ACCESSION NUMBER: TrEMBL/Q8WU90
  ref <309>: DATABASE ENTRY DATE: 2002-03-01
  ref <313>: RELEVANT RESIDUES IN SEQ ID NO: (1)..(426)

SEQ ID NO: 46 - protein, 426 aa, Homo sapiens [sequence omitted]

SEQ ID NO: 47 - protein, 413 aa, Homo sapiens
  ref <308>: DATABASE ACCESSION NUMBER: TrEMBL/Q5QTQ4
  ref <309>: DATABASE ENTRY DATE: 2005-01-04
  ref <313>: RELEVANT RESIDUES IN SEQ ID NO: (1)..(413)
MPPKKQAQAGGSKKAEQKKKEKIIEDKTFGLKNKKGAKQQKFIKAVTHQVKFGQQNPRQVAQSEAEKKLKKDDKKKELQELNELFKPVVAAQKISKGADPKSVVCAFFKQGQCTKGDKCKFSHDLTLERKCEKRSVYIDARDEELEKDTMDNWDEKKLEEVVNKKHGEAEKKKPKTQIVCKHFLEAIENNKYGWFWVCPGGGDICMYRHALPPGFVLKKDKKKEEKEDEISLEDLIERERSALGPNVTKITLESFLAWKKRKRQEKIDKLEQDMERRKADFKAGKALVISGREVFEFRPELVNDDDEEADDTRYTQGTGGDEVDDSVSVNDIDLSLYIPRDVDETGITVASLERFSTYTSDKDENKLSEASGGRAENGERSDLEEDNEREGTENGAIDAVPVDEKSFHWRGFG

SEQ ID NO: 48 - protein, 397 aa, Homo sapiens
  ref <308>: DATABASE ACCESSION NUMBER: TrEMBL/Q9NZ37
  ref <309>: DATABASE ENTRY DATE: 2000-10-01
  ref <313>: RELEVANT RESIDUES IN SEQ ID NO: (1)..(397)
MPPKKQAQAGGSKKADEKKKEKIIEDKTFGLKNKKGAKQQKFIKAVTHQVKFGQQNPRQVAQSEAEKKLKKDDKKKDLQELNELFKPVVAAQKISKGADPKSVVCAFFKQGQCTKGDKCKFSHDLTLERKCEKRSVYIDARDEELEKDTMDNWDEKKLEEVVNKKHGEAEKKKPKTQIVCKHFLEAIENNKYGWFWVCPGGGDICMYRHALPPGFVLKKDKKKEEKEDEISLEDLIERERSALGPNVTKITLESFLAWKKRKRQEKIDKLEQDMERRKADFKAGKALVISGREVFEFRPELVNDDDEEADDTRYTQGTGGDEVDDSVSVNDIDLSLYIPRDVDETGITVASLERFSTYTSDKDENKLSEASGGRAENGERSDLEEDNEREGTGKWSH

SEQ ID NO: 49 - protein, 426 aa, Rattus norvegicus
  ref <308>: DATABASE ACCESSION NUMBER: UniProtKB/Q6U6G5
  ref <309>: DATABASE ENTRY DATE: 2004-07-05
  ref <313>: RELEVANT RESIDUES IN SEQ ID NO: (1)..(426)
MPPKKQAQAGGSKKAEQKKKEKIIEDKTFGLKNKKGAKQQKFIKAVTHQVKFGQQNPRQVAQSEAEKKLKKDDKKKELQELNELFKPVVAAQKISKGADPKSVVCAFFKQGQCTKGDKCKFSHDLTLERKCEKRSVYIDARDEELEKDTMDNWDEKKLEEVVNKKHGEAEKKKPKTQIVCRHFLEAIENNKYGWFWVCPGGGDNCMYRHALPPGFVLKKDKKKEEKEDEISLEDLIERERSALGPNVTKITLESFLAWKKRKRQEKIDKLEQDMERRKADFKAGKALVISGREVFEFRPELVNDDDEEADDTRYIQGTGGDEVDDSVGVNDIDLSLYVPRDVEETGITVASLERFSTYASDKDENKLSEASGGLAENGERSDLDEDSGGGGQENGSIDAVPVDENLFTGEDLDELEEELNTLDLEE

SEQ ID NO: 50 - protein, 426 aa, Mus musculus [sequence omitted]

SEQ ID NO: 51 - protein, 426 aa, Mus musculus
  ref <308>: DATABASE ACCESSION NUMBER: UniProtKB/Q3TWQ0
  ref <309>: DATABASE ENTRY DATE: 2005-10-11
  ref <313>: RELEVANT RESIDUES IN SEQ ID NO: (1)..(426)
MPPKKQAQAGGSKKAEQKKKEKIIEDKTFGLKNKKGAKQQKFIKAVTHQVKFGQQNPRQVAQSEAEKKLKKDDKKKELQELNELFKPVVAAQKISKGADPKSVVCAFFKQGQCTKGDKCKFSHDLTLERKCEKRSVYIDAREEELEKDTMDNWDEKKLEEVVNKKHGEAEKKKPKTQIVCRHFLEAIENNKYGWFWVCPGGGDNCMYRHALPPGFVLKKDKKKEEKEDEISLEDLIERERSALGPNVTKITLESFLAWKKRKRQEKIDKLEQDMERRKADFKAGKALVISGREVFEFRPELVNDDDEEADDTRYIQGTGGDEVDDSMGVNDIDISLYVPRDVEETGITVASVERFSTYAPDKDENKLSEASGGLAENGERSDLDEDSGGGGQENGSIDAVPVDENLFTGEDLDELEEELNTLDLEE

SEQ ID NO: 52 - protein, 426 aa, Mus musculus
  ref <308>: DATABASE ACCESSION NUMBER: UniProtKB/Q3TIV5
  ref <309>: DATABASE ENTRY DATE: 2005-10-11
  ref <313>: RELEVANT RESIDUES IN SEQ ID NO: (1)..(426)
MPPKKQAQAGGSKKAEQKKKEKIIEDKTFGLKNKKGAKQQKFIKAVTHQVKFGQQNPRQVAQSEAEKKLKKDDKKKELQELNELFKPVVAAQKISKGADPKSVVCAFFKQGQCTKGDKCKFSHDLTLERKCEKRSVYIDARDEEPEKDTMDNWDEKKLEEVVNKKHGEAEKKKPKTQIVCRHFLEAIENNKYGWFWVCPGGGDNCMYRHALPPGFVLKKDKKKEEKEDEISLEDLIERERSALGPNVTKITLESFLAWKKRKRQEKIDKLEQDMERRKADFKAGKALVISGREVFEFRPELVNDDDEEADDTRYIQGTGGDEVDDSMGVNDIDISLYVPRDVEETGITVASVERFSTYAPDKDENKLSEASGGLAENGERSDLDEDSGGGGQENGSIDAVPVDENLFTGEDLDELEEELNTLDLEE

SEQ ID NO: 53 - protein, 401 aa, Mus musculus
  ref <308>: DATABASE ACCESSION NUMBER: UniProtKB/Q3U855
  ref <309>: DATABASE ENTRY DATE: 2005-10-11
  ref <313>: RELEVANT RESIDUES IN SEQ ID NO: (1)..(401)
MPPKKQAQAGGSKKAEQKKKEKIIEDKTFGLKNKKGAKQQKFIKAVTHQVKFGQQNPRQVAQSEAEKKLKKDDKKKELQELNELFKPVVAAQKISKGADPKSVVCAFFKQGQCTKGDKCKFSHDLTLERKCEKRSVYIDARDEELEKDTMDNWDEKKLEEVVNKKHGEAEKKKPKTQIVCRHFLEAIENNKYGWFWVCPGGGDNCMYRHALPPGFVLKKDKKKEEKEDEISLEDLIERERSALGPNVTKITLESFLAWKKRKRQEKIDKLEQDMERRKADFKAGKALVISGREVFEFRPELVNDDDEEADDTRYIQGTGGDEVDDSMGVNDIDISLYVPRDVEETGITVASVERFSTYAPDKDENKLSEASGGLAENGERSDLDEDSGGGGQKWIHRCCSC

SEQ ID NO: 54 - protein, 429 aa, Gallus gallus
  ref <308>: DATABASE ACCESSION NUMBER: UniProtKB/Q5H7N8
  ref <309>: DATABASE ENTRY DATE: 2005-03-01
  ref <313>: RELEVANT RESIDUES IN SEQ ID NO: (1)..(429)
MPPKKQQQPAGSSKKADQKKKEKIIEDKTFGLKNKKGAKQQKFFKAVTHLVKFGQQYPRQAAQTESEKKLKKEDKKKELQELNELFKPVVAAQKISKGADPKSVVCAFFKQGQCTKGDKCKFSHDLSLERKCEKRSVYIDARDEDLEKDTMDNWDEKKLEEVVNKKHGEAEKKKPKTQIVCKYFLDAIENNKYGWFWVCPGGGDNCMYRHALPPGFVLKKDKKKEEKQDEISLEDLIEKERAALGPNVTKITLECFIAWKRRKRQEKIDKAEQDMERRKADFKAGKALVISGREVFEFRPELVDADDEEADDTRYTQGTGEDDEVEDPVCVNDVDLSLYVPKAVDETGITVASPERFSTYASIEKDDNKLSEASGGEINSSEQNDLEDRDGDEELENGVMDAVPVDENLFTGEDLDELEEELNTLDLEE

SEQ ID NO: 55 - protein, 151 aa, Mus musculus
  ref <308>: DATABASE ACCESSION NUMBER: TrEMBL/Q9DAP2
  ref <309>: DATABASE ENTRY DATE: 2001-06-01
  ref <313>: RELEVANT RESIDUES IN SEQ ID NO: (1)..(151)
MPPKKQAQAGGSKKAEQKKKEKIIEDKTFGLKNKKGAKQQKFIKAVTHQVKFGQQNPRQVAQSEAEKKLKKDDKKKELQELNELFKPVVAAQKISKGADPKSVVCAFFKQGQCTKGDKCKFSHDLTLERKCEKRSVYIDARDEELEKGISS

SEQ ID NO: 56 - protein, 272 aa, Bos taurus
  ref <308>: DATABASE ACCESSION NUMBER: UniProtKB/Q3T0Q9
  ref <309>: DATABASE ENTRY DATE: 2005-10-11
  ref <313>: RELEVANT RESIDUES IN SEQ ID NO: (1)..(272)
MSDSLSRRVTFAVTAWMLSVVSSSSAPANTLPDIENEDFIKDCVRMHNKFRSSVTPAASDMLYMTWDPLLAQIAKAWASHCEFAHNKRLKPPYKLHPNFTSLGENLWTGSLSIFSVSSAITAWYDEVKYYDFKTRKCNKVCGHYTQVVWADSYKVGCAVHFCPRVSGFGALLNGAHFICNYGPPGNYPTWPYKKGSTCSACPRNDKCLDNLCTNPQRDKVTRYYSTVFPDWPIFPRDRYTSLFLIIIPPILILSVIIIIWAKGKYPHLFTLK

SEQ ID NO: 57 - protein, 255 aa, Mus musculus
  ref <308>: DATABASE ACCESSION NUMBER: GenBank/Q9CWG1
  ref <309>: DATABASE ENTRY DATE: 2005-01-04
  ref <313>: RELEVANT RESIDUES IN SEQ ID NO: (1)..(255)
MQVILAVIVWMASSVSSSSFTASTLPDITNEDFIKECVQVHNQLRSKVSPPARNMLYMSWDPKLAQIAKAWTKSCEFKHNPQLHSRIHPNFTALGENIWLGSLSIFSVSSAISAWYEEIKHYDFSTRKCRHVCGHYTQVVWADSYKLGCAVQLCPNGANFICDYGPAGNYPTWPYKQGATCSDCPKDDKCLNSLCINPRRDQVSRYYSVDYPDWPIYLRNRYTSLFLIAKSVLLLLSVIITIWVKHKYPNLVLLD

SEQ ID NO: 58 - protein, 255 aa, Mus musculus; identical to SEQ ID NO: 57
  ref <308>: DATABASE ACCESSION NUMBER: TrEMBL/Q4QQK5
  ref <309>: DATABASE ENTRY DATE: 2005-07-19
  ref <313>: RELEVANT RESIDUES IN SEQ ID NO: (1)..(255)

SEQ ID NO: 59 - protein, 251 aa, Rattus norvegicus
  ref <308>: DATABASE ACCESSION NUMBER: TrEMBL/Q5FVN7
  ref <309>: DATABASE ENTRY DATE: 2005-03-01
  ref <313>: RELEVANT RESIDUES IN SEQ ID NO: (1)..(251)
MQVLLAVMVWMASSASGFSYTASTLPKITNEDFIEECVEVHNHFRSKAYPPAGNMLYMSWDPKLAQIAKAWAQSCVFQHNPQLHSRIHPNFTGLGENIWLGSLSLFSVRAAILAWFEESQYYDFSTGKCKKVCGHYTQIVWADSYKIGCAVQLCPRGANFICNYGPAGNYPTWPYKQGATCSACPKDDKCLNNLCTNPQRDQVSRHSADYPKYLRNRYTSLYLIAKSVLLLLSVIITIWVKHKYPNLVLLD

SEQ ID NO: 60 - protein, 241 aa, Bos taurus
  ref <308>: DATABASE ACCESSION NUMBER: GenBank/Q32LB5
  ref <309>: DATABASE ENTRY DATE: 2007-01-13
  ref <313>: RELEVANT RESIDUES IN SEQ ID NO: (1)..(241)
MILRKKLSYLWTLGLCLVASKSPPKAPSITNDRFIEECLRLHNEARTNVSPPAADMKYMSWDEALAKTAEAWAKKCKFIHNSCSSKSFKCHPTFQYAGENLWLGPLTISAAKFAINMWYDERKFYDFNTRSCSQVCGHYTQVVWAYSYKVGCAVAVCPNLGSPDSALLVCNYAPAGNYPNMSPYTNGTPCSMCQGDTCENNLCRNKERDKSQRYPNWNPSGTRQLIACNPLYLISVLLTIF

SEQ ID NO: 61 - protein, 179 aa, Gallus gallus
  ref <308>: DATABASE ACCESSION NUMBER: TrEMBL/Q5F3D6
  ref <309>: DATABASE ENTRY DATE: 2005-03-15
  ref <313>: RELEVANT RESIDUES IN SEQ ID NO: (1)..(179)
MISRLFTCVLPLLHFCLSSGLYQPKTLPDIGDAEFIEECVRTHNGFRSGVNPPASNMLYMSWDPDLAKTARGWAKRCEFKHNIYLQEPGQAHPRFTSVGENLWTGSLSIFSVQDAITSWYKEVRNYTYTTNSCSRICGHYTQVVWAQSYKVGCAVHFCPIVSYFSGTNAAHFVCNYGPA

SEQ ID NO: 62 - protein, 312 aa, Homo sapiens
  ref <308>: DATABASE ACCESSION NUMBER: TrEMBL/Q4G1C9
  ref <309>: DATABASE ENTRY DATE: 2005-08-30
  ref <313>: RELEVANT RESIDUES IN SEQ ID NO: (1)..(312)
MEAARPFAREWRAQPLPLAVGGVLKLRLCELWLLLLGSSLNARFLPDEEDVDFINEYVNLHNELRGDVIPRGSNLRFMTWDVALSRTARAWGKKCLFTHNIYLQDVQMVHPKFYGIGENMWVGPENEFTASIAIRSWHAEKKMYNFENGSCSGDCSNYIQLVWDHSYKVGCAVTPCSKIGHIIHAAIFICNYAPGGTLTRRPYEPGIFCTRCGRRDKCTDFLCSNADRDQATYYRFWYPKWEMPRPVVCDPLCTFILLLRILCFILCVITVLIVQSQFPNILLEQQMIFTPEESEAGNEEEEKEEEKKKKKK

SEQ ID NO: 63 - protein, 242 aa, Homo sapiens
  ref <308>: DATABASE ACCESSION NUMBER: GenBank/Q6UWM5
  ref <309>: DATABASE ENTRY DATE: 2007-01-23
  ref <313>: RELEVANT RESIDUES IN SEQ ID NO: (1)..(242)
MALKNKFSCLWILGLCLVATTSSKIPSITDPHFIDNCIEAHNEWRGKVNPPAADMKYMIWDKGLAKMAKAWANQCKFEHNDCLDKSYKCYAAFEYVGENIWLGGIKSFTPRHAITAWYNETQFYDFDSLSCSRVCGHYTQLVWANSFYVGCAVAMCPNLGGASTAIFVCNYGPAGNFANMPPYVRGESCSLCSKEEKCVKNLCRTPQLIIPNQNPFLKPTGRAPQQTAFNPFSLGFLLLRIF

SEQ ID NO: 64 - protein, 310 aa, Mus musculus
  ref <308>: DATABASE ACCESSION NUMBER: TrEMBL/Q3TTM5
  ref <309>: DATABASE ENTRY DATE: 2005-10-11
  ref <313>: RELEVANT RESIDUES IN SEQ ID NO: (1)..(310)
MKASLPWSVVWRAQSNYVRLRRVLKLCELWLLLVGSGLNAKLPLEEDVDFINEYVGLHNELRGTVFPPGVNLRFMTWDVALSRTARAWGKKCMYSRNTHLDKLHESHPVFTEIGENMWVGPVEDFTVTTAIRSWHEERKSYSYLNDTCVEDQNCSHYIQLVWDSSYKVGCAVTSCARAGGFTHAALFICNYAPGGTLTRRPYQAGQFCSRCGPGDQCTDYLCSNTVRDEATYYQFWYPPWEKPRPVVCNPMCIFILFLRVASLLLCVIVVLIVQSRFPVILMETPTIISAEEEGKTEVEIVMEEGEGEGE

SEQ ID NO: 65 - protein, 332 aa, Mus musculus
  ref <308>: DATABASE ACCESSION NUMBER: TrEMBL/Q9CQ35
  ref <309>: DATABASE ENTRY DATE: 2001-06-01
  ref <313>: RELEVANT RESIDUES IN SEQ ID NO: (1)..(332)
MKASLPWSVVWRAQSNYVRLRRVLKLCELWLLLVGSGLNAKLPLEEDVDFINEYVGLHNELRGTVFPPGVNLRFMTWDVALSRTARAWGKKCMYSRNTHLDKLHESHPVFTEIGENMWVGPVEDFTVTTAIRSWHEERKSYSYLNDTCVEDQNCSHYIQLVWDSSYKVGCAVTSCARAGGFTHAALFICNYAPGGTLTRRPYQAGQFCSRCGPGDQCTDYLCSNTVRDEATYYQFWYPPWEKPRPVVCNPMCIFILFLRVASLLLCVIVVLIVQSRFPVILMETPTIISAEEEGKTEVEIVMEEGEGEGEGGEGEGEGEEKEEEEMLEEDEQ

SEQ ID NO: 66 - protein, 229 aa, Homo sapiens
  ref <308>: DATABASE ACCESSION NUMBER: TrEMBL/Q8NA43
  ref <309>: DATABASE ENTRY DATE: 2002-10-01
  ref <313>: RELEVANT RESIDUES IN SEQ ID NO: (1)..(229)
MEAARPFAREWRAQSLPLAVGGVLKLRLCELWLLLLGSSLNARFLPDEEDVDFINEYVNLHNELRGDVIPRGSNLRFMTWDVALSRTARAWGKKCLFTHNIYLQDVQMVHPKFYGIGENMWVGPENEFTASIAIRSWHAEKKMYNFENGSCSGDCSNYIQLVWDHSYKVGCAVTPCSKIGHIIHAAIFICNYAPGGTLTRRPYEPGIFCTRCGRRDKCTDFLCSNADRD

SEQ ID NO: 67 - protein, 253 aa, Homo sapiens
  ref <308>: DATABASE ACCESSION NUMBER: TrEMBL/Q8N6N0
  ref <309>: DATABASE ENTRY DATE: 2002-10-01
  ref <313>: RELEVANT RESIDUES IN SEQ ID NO: (1)..(253)
MEAARPFAREWRAQSLPLAVGGVLKLRLCELWLLLLGSSLNARFLPDEEDVDFINEYVNLHNELRGDVIPRGSNLRFMTWDVALSRTARAWGKKCLFTHNIYLQDVQMVHPKFYGIGENMWVGPENEFTASIAIRSWHAEKKMYNFENGSCSGDCSNYIQLVWDHSYKVGCAVTPCSKIGHIIHAAIFICNYAPGGTLTRRPYEPGIFCTRCGRRDKCTDFLCSKIKKINMKKMHNGLDKKNKRLNTSFLWSC

SEQ ID NO: 68 - protein, 236 aa, Mus musculus
  ref <308>: DATABASE ACCESSION NUMBER: TrEMBL/Q9DAG6
  ref <309>: DATABASE ENTRY DATE: 2001-06-01
  ref <313>: RELEVANT RESIDUES IN SEQ ID NO: (1)..(236)
MALKKKLNFLWTLVLYLIASRLPKAFGKDLPRVPTITDPKFIDAFLNIHNELRRKVQPPAADMNQLFWDQQLAKLAKAWTRECKLAHNPCIKQRYECLEDYDFIGENIYLGRIETQPEDVVINWYNESKYFNFDFNTCSEMCGHYTQVVWAKTVKIGCAVSNCPNLKGFSAGLFVCNYSPAGNFIGFRPYTRGDSCSMCGQKTCENSLCRPMNRKTPHHKAACHVLVLGFILQSLL

SEQ ID NO: 69 - protein, 371 aa, Homo sapiens
  ref <308>: DATABASE ACCESSION NUMBER: TrEMBL/Q96K61
  ref <309>: DATABASE ENTRY DATE: 2001-12-01
  ref <313>: RELEVANT RESIDUES IN SEQ ID NO: (1)..(371)
MSCVLGGVIPLGLLFLVCGSQGYLLPNVTLLEELLSKYQHNESHSRVRRAIPREDKEEILMLHNKLRGQVQPQASNMEYMTWDDELEKSAAAWASQCIWEHGPTSLLVSIGQNLGAHWGRYRSPGFHVQSWYDEVKDYTYPYPSECNPWCPERCSGPMCTHYTQIVWATTNKIGCAVNTCRKMTVWGEVWENAVYFVCNYSPKGNWIGEAPYKNGRPCSECPPSYGGSCRNNLCYREETYTPKPETDEMNEVETAPIPEENHVWLQPRVMRPTKPKKTSAVNYMTQVVRCDTKMKDRCKGSTCNRYQCPAGCLNHKAKIFGTLFYESSSSICRAAIHYGILDDKGGLVDITRNGKVPFFVKSERHGVQSLR

SEQ ID NO: 70 - protein, 497 aa, Homo sapiens [sequence omitted]

SEQ ID NO: 71 - protein, 449 aa, Homo sapiens [sequence omitted]

SEQ ID NO: 72 - protein, 500 aa, Homo sapiens
  ref <308>: DATABASE ACCESSION NUMBER: SwissProt/Q9H336
  ref <309>: DATABASE ENTRY DATE: 2006-09-05
  ref <313>: RELEVANT RESIDUES IN SEQ ID NO: (1)..(500)
MKCTAREWLRVTTVLFMARAIPAMVVPNATLLEKLLEKYMDEDGEWWIAKQRGKRAITDNDMQSILDLHNKLRSQVYPTASNMEYMTWDVELERSAESWAESCLWEHGPASLLPSIGQNLGAHWGRYRPPTFHVQSWYDEVKDFSYPYEHECNPYCPFRCSGPVCTHYTQVVWATSNRIGCAINLCHNMNIWGQIWPKAVYLVCNYSPKGNWWGHAPYKHGRPCSACPPSFGGGCRENLCYKEGSDRYYPPREEETNEIERQQSQVHDTHVRTRSDDSSRNEVISAQQMSQIVSCEVRLRDQCKGTTCNRYECPAGCLDSKAKVIGSVHYEMQSSICRAAIHYGIIDNDGGWVDITRQGRKHYFIKSNRNGIQTIGKYQSANSFTVSKVTVQAVTCETTVEQLCPFHKPASHCPRVYCPRNCMQANPHYARVIGTRVYSDLSSICRAAVHAGVVRNHGGYVDVMPVDKRKTYIASFQNGIFSESLQNPPGGKAFRVFAVV

SEQ ID NO: 73 - protein, 489 aa, Mus musculus
  ref <308>: DATABASE ACCESSION NUMBER: SwissProt/Q9JJ56
  ref <309>: DATABASE ENTRY DATE: 2001-10-01
  ref <313>: RELEVANT RESIDUES IN SEQ ID NO: (1)..(489)
MLPPPLLLLLLLIATGPTTALTEDEKQTMVDLHNQYRAQVSPPASDMLQMRWDDELAAFAKAYAQKCVWGHNKERGRRGENLFAITDEGMDVPLAVGNWHEEHEYYNFSTATCDPNQMCGHYTQVVWSKTERIGCGSHFCETLQGVEEANIHLLVCNYEPPGNVKGRKPYQEGTPCSQCPLGYSCENSLCEPMRNPEKAQDSPPRVTEVPSTRATEAPSSRETGTPSLATSETLHFSVIKVSDSLATESSPAVETKAPSSLATEGPSSMATEAQAFVTEVPLVSARHMQPSVDEGPVNFLTSTHIPVPKSMDEEASKSSATSVSPKKSLYPKMSLTESGESVPQIQEEAEPKDELSEPEAILPEAEAAPTEAEVELREPEAESPKAESPEAEAESPLSSEALVPVLPAQERGGQKASLEHSGHPASPSLPTFPSASGNATGGRTLALQSSWTGAENPEKADWDLKNSAHVWGPFLGLLLPSLLLLAGMV

SEQ ID NO: 74 - protein, 371 aa, Artificial
  putative dominant negative mutant of LEREPO4
MPPKKQAQAGGSKKAEQKKKEKIIEDKTFGLKNKKGAKQQKFIKAVTHQVKFGQQNPRQVAQSEAEKKLKKDDKKKELQELNELFKPVVAAQKISKGADTLERKCEKRSVYIDARDEELEKDTMDNWDEKKLEEVVNKKHGEAEKKKPKTQIVCKHFLEAIENNKYGWFWVCPGGGDICMYRHALPPGFVLKKDKKKEEKEDEISLEDLIERERSALGPNVTKITLESFLAWKKRKRQEKIDKLEQDMERRKADFKAGKALVISGREVFEFRPELVNDDDEEADDTRYTQGTGGDEVDDSVSVNDIDLSLYIPRDVDETGITVASLERFSTYTSDKDENKLSEASGGRAENGERSDLEEDNEREGTENGAI

SEQ ID NO: 75 - protein, 397 aa, Artificial
  putative dominant negative mutant of LEREPO4
  misc_feature (105)..(105): Xaa can be any naturally occurring amino acid
  misc_feature (113)..(113): Xaa can be any naturally occurring amino acid
  misc_feature (119)..(119): Xaa can be any naturally occurring amino acid
MPPKKQAQAGGSKKAEQKKKEKIIEDKTFGLKNKKGAKQQKFIKAVTHQVKFGQQNPRQVAQSEAEKKLKKDDKKKELQELNELFKPVVAAQKISKGADPKSVVXAFFKQGQXTKGDKXKFSZDLTLERKCEKRSVYIDARDEELEKDTMDNWDEKKLEEVVNKKHGEAEKKKPKTQIVCKHFLEAIENNKYGWFWVCPGGGDICMYRHALPPGFVLKKDKKKEEKEDEISLEDLIERERSALGPNVTKITLESFLAWKKRKRQEKIDKLEQDMERRKADFKAGKALVISGREVFEFRPELVNDDDEEADDTRYTQGTGGDEVDDSVSVNDIDLSLYIPRDVDETGITVASLERFSTYTSDKDENKLSEASGGRAENGERSDLEEDNEREGTENGAI

SEQ ID NO: 76 - protein, 397 aa, Artificial
  putative dominant negative mutant of LEREPO4
  misc_feature (105)..(105): Xaa can be any naturally occurring amino acid
MPPKKQAQAGGSKKAEQKKKEKIIEDKTFGLKNKKGAKQQKFIKAVTHQVKFGQQNPRQVAQSEAEKKLKKDDKKKELQELNELFKPVVAAQKISKGADPKSVVXAFFKQGQCTKGDKCKFSHDLTLERKCEKRSVYIDARDEELEKDTMDNWDEKKLEEVVNKKHGEAEKKKPKTQIVCKHFLEAIENNKYGWFWVCPGGGDICMYRHALPPGFVLKKDKKKEEKEDEISLEDLIERERSALGPNVTKITLESFLAWKKRKRQEKIDKLEQDMERRKADFKAGKALVISGREVFEFRPELVNDDDEEADDTRYTQGTGGDEVDDSVSVNDIDLSLYIPRDVDETGITVASLERFSTYTSDKDENKLSEASGGRAENGERSDLEEDNEREGTENGAI

SEQ ID NO: 77 - protein, 397 aa, Artificial
  putative dominant negative mutant of LEREPO4
  misc_feature (113)..(113): Xaa can be any naturally occurring amino acid
MPPKKQAQAGGSKKAEQKKKEKIIEDKTFGLKNKKGAKQQKFIKAVTHQVKFGQQNPRQVAQSEAEKKLKKDDKKKELQELNELFKPVVAAQKISKGADPKSVVCAFFKQGQXTKGDKCKFSHDLTLERKCEKRSVYIDARDEELEKDTMDNWDEKKLEEVVNKKHGEAEKKKPKTQIVCKHFLEAIENNKYGWFWVCPGGGDICMYRHALPPGFVLKKDKKKEEKEDEISLEDLIERERSALGPNVTKITLESFLAWKKRKRQEKIDKLEQDMERRKADFKAGKALVISGREVFEFRPELVNDDDEEADDTRYTQGTGGDEVDDSVSVNDIDLSLYIPRDVDETGITVASLERFSTYTSDKDENKLSEASGGRAENGERSDLEEDNEREGTENGAI

SEQ ID NO: 78 - protein, 397 aa, Artificial
  putative dominant negative mutant of LEREPO4
  misc_feature (119)..(119): Xaa can be any naturally occurring amino acid
MPPKKQAQAGGSKKAEQKKKEKIIEDKTFGLKNKKGAKQQKFIKAVTHQVKFGQQNPRQVAQSEAEKKLKKDDKKKELQELNELFKPVVAAQKISKGADPKSVVCAFFKQGQCTKGDKXKFSHDLTLERKCEKRSVYIDARDEELEKDTMDNWDEKKLEEVVNKKHGEAEKKKPKTQIVCKHFLEAIENNKYGWFWVCPGGGDICMYRHALPPGFVLKKDKKKEEKEDEISLEDLIERERSALGPNVTKITLESFLAWKKRKRQEKIDKLEQDMERRKADFKAGKALVISGREVFEFRPELVNDDDEEADDTRYTQGTGGDEVDDSVSVNDIDLSLYIPRDVDETGITVASLERFSTYTSDKDENKLSEASGGRAENGERSDLEEDNEREGTENGAI

SEQ ID NO: 79 - protein, 397 aa, Artificial
  putative dominant negative mutant of LEREPO4
MPPKKQAQAGGSKKAEQKKKEKIIEDKTFGLKNKKGAKQQKFIKAVTHQVKFGQQNPRQVAQSEAEKKLKKDDKKKELQELNELFKPVVAAQKISKGADPKSVVCAFFKQGQCTKGDKCKFSZDLTLERKCEKRSVYIDARDEELEKDTMDNWDEKKLEEVVNKKHGEAEKKKPKTQIVCKHFLEAIENNKYGWFWVCPGGGDICMYRHALPPGFVLKKDKKKEEKEDEISLEDLIERERSALGPNVTKITLESFLAWKKRKRQEKIDKLEQDMERRKADFKAGKALVISGREVFEFRPELVNDDDEEADDTRYTQGTGGDEVDDSVSVNDIDLSLYIPRDVDETGITVASLERFSTYTSDKDENKLSEASGGRAENGERSDLEEDNEREGTENGAI

SEQ ID NO: 80 - protein, 245 aa, Artificial
  putative dominant negative mutant of GLIPR
ANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRYTSLFLIVNSVILILSVIITILVQHKYPNLVLLD

SEQ ID NO: 81 - protein, 266 aa, Artificial
  putative dominant negative mutant of GLIPR
MRVTLATIAWMVSFVSNYSHTANILPDIENEDFIKDCVRIZNKFRSEVKPTASDMLYMTWDPALAQIAKAWASNCQFSZNTRLKPPHKLHPNFTSLGENIWTGSVPIFSVSSAITNWYDEIQDYDFKTRICKKVCGZYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRYTSLFLIVNSVILILSVIITILVQHKYPNLVLLD

SEQ ID NO: 82 - protein, 266 aa, Artificial
  putative dominant negative mutant of GLIPR
MRVTLATIAWMVSFVSNYSHTANILPDIENEDFIKDCVRIZNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRYTSLFLIVNSVILILSVIITILVQHKYPNLVLLD

SEQ ID NO: 83 - protein, 266 aa, Artificial
  putative dominant negative mutant of GLIPR
MRVTLATIAWMVSFVSNYSHTANILPDIENEDFIKDCVRIHNKFRSEVKPTASDMLYMTWDPALAQIAKAWASNCQFSZNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRYTSLFLIVNSVILILSVIITILVQHKYPNLVLLD

SEQ ID NO: 84 - protein, 266 aa, Artificial
  putative dominant negative mutant of GLIPR
MRVTLATIAWMVSFVSNYSHTANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRYTSLFLIVNSVILILSVIITILVQHKYPNLVLLD

SEQ ID NO: 85 - protein, 266 aa, Artificial
  putative dominant negative mutant of GLIPR
MRVTLATIAWMVSFVSNYSHTANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGZYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRYTSLFLIVNSVILILSVIITILVQHKYPNLVLLD

SEQ ID NO: 86 - protein, 255 aa, Artificial
  putative dominant negative mutant of GLIPR
MRVTLATIAWMVSFVSNYSHTANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCYKVGCAVQFCPKVSGFDALSNGAHFICNYGPGGNYPTWPYKRGATCSACPNNDKCLDNLCVNRQRDQVKRYYSVVYPGWPIYPRNRYTSLFLIVNSVILILSVIITILVQHKYPNLVLLD

SEQ ID NO: 87 - protein, 254 aa, Artificial
  putative dominant negative mutant of GLIPR
MRVTLATIAWMVSFVSNYSHTANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAPTWPYKRGATCSACPNNDKCLDNLCVNRQRDQVKRYYSVVYPGWPIYPRNRYTSLFLIVNSVILILSVIITILVQHKYPNLVLLD

SEQ ID NO: 88 - protein, 232 aa, Artificial
  putative dominant negative mutant of GLIPR
MRVTLATIAWMVSFVSNYSHTANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNR

(19) United States
(12) Patent Application Publication
(10) Pub. No.: US 2009/0233985 A1
(43) Pub. Date: Sep. 17, 2009

(54) COMPOSITIONS AND METHODS FOR INHIBITING HIV INFECTIONS BY INHIBITING LEREPO4 AND GLIPR

(76) Inventor: Urban Scheuring, Siegburg (DE)

Correspondence Address: KNOBBE MARTENS OLSON & BEAR LLP, 2040 MAIN STREET, FOURTEENTH FLOOR, IRVINE, CA, 92614, US

(21) Appl. No.: 12/373,282

(22) PCT Filed: Jul. 11, 2007
(86) PCT No.: PCT/EP07/57107

§ 371 Date: Jan. 9, 2009

(30) Foreign Application Priority Data
Jul. 11, 2006 (EP) 06116962.9

(51) Int. Cl. A61K 31/7105; C07H 21/02; C07H 21/04; A61P 31/18; C12N 5/06; C12Q 1/02

(52) U.S. Cl. 514/44 A; 536/24.5; 536/23.5; 435/375; 435/29

(57) ABSTRACT

The present invention relates to inhibitor molecules of LEREPO4 or GliPR or respective functional homologues thereof including siRNAs, shRNAs, antisense RNAs, antisense DNA and dominant negative proteinaceous mutants of LEREPO4 or functional homologues thereof. The present invention also relates to pharmaceutical compositions and methods for preventing and/or inhibiting HIV infections by inhibiting the function of LEREPO4 or GliPR or respective functional ho mo logues thereof in vivo. Furthermore, the present invention relates to methods of treating, preventing or diagnosing AIDS and/or HIV infections in an individual. Moreover, the present invention relates to diagnostic methods to determine the susceptibility of HIV strains and isolates for such LEREPO4 or GliPR directed treatment.

### FIELD OF THE INVENTION

The present invention relates to inhibition and diagnostic characterisation of human immunodeficiency virus (HIV) infections and thus to the diagnosis, prevention and treatment of acquired immunodeficiency syndrome (AIDS).

The present invention relates in particular to inhibitor molecules of the genes or gene products of LEREPO4 or GliPR or their respective functional homologues including siRNAs, shRNAs, antisense RNAs, antisense DNA and dominant negative proteinaceous mutants of LEREPO4 or GliPR or functional homologues thereof, respectively.

The present invention also relates to pharmaceutical compositions and methods for diagnosing, preventing and/or inhibiting HIV infections or HIV replication by inhibiting the function of LEREPO4 or GliPR or their respective functional homologues in vivo.

Furthermore, the present invention relates to methods of treating, preventing or diagnosing AIDS and/or HIV infections in an individual.

In addition, the invention also relates to methods of identifying further inhibitory molecules of LEREPO4 or GliPR or their respective homologues.

Moreover, the invention relates to diagnosing, preventing, treating and/or inhibiting other retroviral infections and associated diseases, including, but not limited to human T-lymphotropic virus I (HTLV-I) and HTLV-II.

### BACKGROUND OF THE INVENTION

The primary cause of Acquired Immunodeficiency Syndrome (AIDS) has been shown to be human immunodeficiency virus (HIV) (Barre-Sinoussi et al., Science, 220, 868-870, 1983; Gallo et al., Science, 224, 500-503, 1984). HIV causes immunodeficiency in an individual by infecting important cell types of the immune system which results in their ultimate depletion. This, in turn leads to opportunistic infections, neoplastic growth and death.

HIV is a member of the lentivirus family of retroviruses (Teich et al., in “RNA tumor viruses”, Weiss et al. (eds.) Cold Spring Harbour Press, 949-956, 1984). Retroviruses are small enveloped viruses that contain a diploid, single-stranded RNA genome and replicate via a DNA intermediate produced by a virally encoded reverse transcriptase, nRNA-dependent DNA polymerase (Varmus, Science, 240, 1427-1439, 1988). There are at least two distinct subtypes of HIV, namely HIV-1 (Barre-Sinoussi et al. vide supra.; Gallo et al., vide supra.) and HIV-2 (Clavel et al., Science, 233, 343-346, 1986; Guyader et al., Nature, 326, 662-669, 1987). There is genetic heterogeneity within each of these HIV subtypes.

Attempts to treat HIV infections have focussed on the development of drugs that disrupt the viral infection and replication cycle (Mitsuya et al., Faseb J., 5, 2369-2381, 1991). Such intervention approaches inhibit the binding of HIV to cell membranes, the entry of HIV into the host cell, the reverse transcription of the HIV RNA genome into DNA, the processing of the virus-specific factors and the exit of the virus from the host cell (see e.g. U.S. Pat. No. 6,475,491).

Thus, virally encoded reverse transcriptase has been a major focus of drug development and a number of reverse-transcriptase-targeted drugs including 2′,3′-deoxynucleotide analogues such as AZT, ddI, ddC and ddT have been shown to be active against HIV (Mitsuya et al., Science, 249, 1533-1544, 1990). Further, more virus-specific drugs than the aforementioned compounds have become available in the meantime.

However, although a considerable amount of effort has also been expanded on the design of effective therapeutics, no curative anti-retroviral drugs against AIDS currently exist. Moreover, most available therapies are hampered by substantial adverse side-effects and HIV's capacity to rapidly mutate into forms that can escape treatment schedules.

Thus, there is a continuing need for safe and effective approaches to prevent and/or treat HIV infections and the development of AIDS.

The object of the present invention is thus to provide further compounds, pharmaceutical compositions and methods for diagnosing, treating and/or preventing HIV infections and the development of AIDS.

### SUMMARY OF THE INVENTION

The experiments set out below reveal that HIV infections lead to increased expression of LEREPO4 and GliPR. A subsequent analysis surprisingly revealed that down-regulation of the host-specific factors LEREPO4 and/or GliPR significantly reduces HIV's replication and/or infective potential.

This invention thus relates to methods and compositions for the prevention, treatment and/or diagnosis of HIV replication in host cells or HIV infection of host cells. In view of the causative dependency of AIDS on HIV infection, the invention also relates to methods and compositions for the prevention, treatment and/or diagnosis of AIDS.

One object of the invention is an inhibitor molecule of LEREPO4 function or functional homologues thereof selected from the group of molecules comprising
(i) recombinant nucleic acid molecules comprising a nucleic acid sequence being complementary and/or specific to the complete coding sequence or parts thereof of LEREPO4 or functional homologues thereof,
(ii) a recombinant nucleic acid molecule encoding a dominant-negative proteinaceous mutant of LEREPO4 or part of it or functional homologues thereof.

In some embodiments, the present invention thus relates to recombinant nucleic acid molecules which are, or encode for, small inhibitory RNA (siRNA) microRNAs (miRNA), antisense mRNA, targeted ribonuclease P molecules, aptamers, antisense DNA or other nucleic acid molecules including those with modified backbone that due to their complementarity or specificity for (parts of) the coding sequence of LEREPO4 or functional homologues thereof or for (parts of) the mRNA sequence of LEREPO4 or functional parts thereof have the capability of interfering with and/or reducing the expression of endogenous LEREPO4 within cells.

One object of the present invention thus pertains to recombinant nucleic acid molecules comprising SEQ ID No. 1 or parts thereof.

In another embodiment of the present invention, the above-mentioned nucleic acid molecules will comprise a contiguous stretch of 10, 11, 12, 13, 14, 15, 16, 17, 18, 19, 20, 21, 22, 23, 24, 25, 26, 27, 28, 29, 30, 50, 70, 100, 150, 200, 300 or more nucleotides of SEQ ID No. 1.

Yet another embodiment of the present invention relates to recombinant nucleic acid comprising nucleic acid molecules which show a degree of identity of at least 30%, at least 35%, at least 40%, at least 45%, at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% with SEQ ID No. 1 over a contiguous stretch of at least 10, 11, 12, 13, 14, 15, 16, 17, 18, 19, 20, 21, 22, 23, 24, 25, 26, 27, 28, 29, 30, 50, 70, 100, 150, 200, 300 or more nucleotides.

In a preferred embodiment, the present invention relates to recombinant nucleic acid molecules comprising SEQ ID Nos. 14, 15 or 16. If these sequences form part of an shRNA, the person skilled in the art is aware that the shRNA will be a double stranded RNA comprising in addition sequences that are fully complementary to the aforementioned sequences, respectively. Another embodiment relates to the nucleic acid molecules encoding for the aforementioned nucleic acid molecules.

One embodiment of the invention relates to recombinant nucleic acid molecules encoding the aforementioned nucleic acid molecule according to (i) as well as the preferred embodiments thereof. Such molecules may e.g. vectors that can be used to transduce mammalian cells and to express the afore-mentioned molecules.

One object of the invention is an inhibitor molecule of GliPR function or functional homologues thereof selected from the group of molecules comprising
(i) recombinant nucleic acid molecules comprising a nucleic acid sequence being complementary and/or specific to the complete coding sequence or parts thereof of GliPR or functional homologues thereof,
(ii) a recombinant nucleic acid molecule encoding a dominant-negative proteinaceous mutant of GliPR or part of it or functional homologues thereof.

In some embodiments, the present invention thus relates to recombinant nucleic acid molecules which are, or encode for, small inhibitory RNA (siRNA) microRNAs (miRNA), antisense mRNA, targeted ribonuclease P molecules, aptamers, antisense DNA or other nucleic acid molecules including those with modified backbone that due to their complementarity or specificity for (parts of) the coding sequence of GliPR or functional homologues thereof or for (parts of) the mRNA sequence of GliPR or functional parts thereof have the capability of interfering with and/or reducing the expression of endogenous GliPR within cells.

One object of the present invention thus pertains to recombinant nucleic acid molecules comprising SEQ ID No. 2 or parts thereof.

In another embodiment of the present invention, the above-mentioned nucleic acid molecules will comprise a contiguous stretch of 10, 11, 12, 13, 14, 15, 16, 17, 18, 19, 20, 21, 22, 23, 24, 25, 26, 27, 28, 29, 30, 50, 70, 100, 150, 200, 300 or more nucleotides of SEQ ID No. 2.

Yet another embodiment of the present invention relates to recombinant nucleic acid comprising nucleic acid molecules which show a degree of identity of at least 30%, at least 35%, at least 40%, at least 45%, at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% with SEQ ID No. 2 over a contiguous stretch of at least 10, 11, 12, 13, 14, 15, 16, 17, 18, 19, 20, 21, 22, 23, 24, 25, 26, 27, 28, 29, 30, 50, 70, 100, 150, 200, 300 or more nucleotides.

In a preferred embodiment, the present invention relates to recombinant nucleic acid molecules comprising SEQ ID Nos. 17, 18 or 19. If these sequences form part of an shRNA, the person skilled in the art is aware that the shRNA will be a double stranded RNA comprising in addition sequences that are fully complementary to the aforementioned sequences, respectively.

One embodiment of the invention relates to recombinant nucleic acid molecules encoding the aforementioned nucleic acid molecule according to (i) as well as the preferred embodiments thereof. Such molecules may e.g. vectors that can be used to transduce mammalian cells and to express the afore-mentioned molecules.

One embodiment of the present invention, as far as recombinant nucleic acid molecules encoding for dominant-negative proteinaceous mutants of LEREPO4 or parts of it or functional homologues thereof are concerned, relate to recombinant nucleic acid molecules comprising SEQ ID No. 3, or parts thereof which carry additional mutations.

Yet another embodiment of the present invention relates to inhibitor molecules of LEREPO4 or parts of it or functional homologues thereof with the inhibitor molecule comprising a polypeptide sequence encoded by a recombinant nucleic acid molecule encoding a dominant-negative proteinaceous mutant of LEREPO4 or functional homologues thereof. Examples of this embodiment can be found in FIGS. 21 and 22.

One embodiment of the present invention, as far as recombinant nucleic acid molecules encoding for dominant-negative proteinaceous mutants of GliPR or parts of it or functional homologues thereof are concerned, relate to recombinant nucleic acid molecules comprising SEQ ID No. 5, or parts thereof which carry additional mutations.

Yet another embodiment of the present invention relates to inhibitor molecules of GliPR or functional homologues thereof with the inhibitor molecule comprising a polypeptide sequence encoded by a recombinant nucleic acid molecule encoding a dominant-negative proteinaceous mutant of GliPR or parts of it or functional homologues thereof. Examples of this embodiment can be found in FIG. 23.

Yet another object of the present invention relates to recombinant nucleic acid molecules such as (virus-based) vectors that can be used to disrupt the endogenous genes of LEREPO4 or functional homologues thereof.

Yet another object of the present invention relates to recombinant nucleic acid molecules such as (virus-based) vectors that can be used to disrupt the endogenous genes of GliPR or functional homologues thereof.

A further object of the present invention concerns pharmaceutical compositions which comprise the above-described inhibitor molecules of LEREPO4 function or functional homologues thereof.

A further object of the present invention concerns pharmaceutical compositions which comprise the above-described inhibitor molecules of GliPR function or functional homologues thereof.

Yet another object of the invention concerns a method of attenuating, reducing or preventing the transmission and/or infection and/or replication of HIV into/in a cell which comprises the provision of the above-described inhibitor molecules and/or pharmaceutical compositions to said cells.

The invention also concerns a method of treating or preventing AIDS in an individual, wherein the method of treatment comprises administering the aforementioned inhibitory molecules of pharmaceutical compositions to HIV-infected, HIV-susceptible or bystander cells of an individual.

A further aspect of the present invention relates to the use of at least one of the aforementioned inhibitor molecules for the manufacture of a medicament in the treatment or prevention of HIV infections and/or AIDS.

The invention also concerns a method of diagnosing AIDS and/or HIV infection in an individual or isolated cells, wherein the method comprises the steps of:
a) obtaining a cellular sample from an individual being potentially afflicted with AIDS and/or a HIV infection;
b) determining the expression level of LEREPO4 and/or GliPR or of their respective functional homologues in said sample outside the individual's body;
c) comparing said expression level of LEREPO4 and/or GliPR or of their respective functional homologues with the expression level of LEREPO4 and/or GliPR or of their respective functional homologues in a sample obtained from an individual not afflicted with AIDS or a HIV infection; and
d) determining the occurrence of AIDS or HIV infection by observing an increased expression level of LEREPO4 and/or GliPR or of their respective functional homologues compared to the sample obtained from an individual not afflicted with AIDS or a HIV infection.
The above method of diagnosis may also be used to determine the susceptibility of an individual to the aforementioned methods of treating and preventing HIV infection/AIDS with said inhibitory molecules of pharmaceutical compositions specific to LEREPO4 or GliPR.
The above method of diagnosis may also be used to determine the susceptibility of a specific HIV type, strain or isolate to the aforementioned methods of treating and preventing HIV infection/AIDS with said inhibitory molecules of pharmaceutical compositions specific to LEREPO4 or GliPR.
The above method of diagnosis may also be used to determine the susceptibility of a specific HIV isolate from a specific individual to the aforementioned methods of treating and preventing HIV infection/AIDS with said inhibitory molecules of pharmaceutical compositions specific to LEREPO4 or GliPR.
The above method of diagnosis may also be used to assess the prognosis or prediction of the therapeutic outcome to the aforementioned methods of treating and preventing HIV infection/AIDS with said inhibitory molecules of pharmaceutical compositions specific to LEREPO4 or GliPR by determining LEREPO4 or GliPR expression or by testing HIV isolate, strain, susceptibility to said therapy in vitro.

The invention similarly relates to a method of data acquisition comprising the steps of:
a) obtaining a cellular sample from an individual being potentially afflicted with AIDS and/or a HIV infection;
b) determining the expression level of LEREPO4 and/or GliPR or of their respective functional homologues in said sample;
c) comparing said expression level of LEREPO4 and/or GliPR or of their respective functional homologues with the expression level of LEREPO4 and/or GliPR or of their respective functional homologues in a sample obtained from an individual not afflicted with AIDS or a HIV infection.

As such, the invention relates to the use of the expression of LEREPO4 and/or GliPR or of their respective functional homologues as an indication of AIDS or HIV infection in an individual or as an indication of the susceptibility of the HIV strain/isolate of this individual to the aforementioned methods of treating and preventing HIV infection/AIDS with said inhibitory molecules of pharmaceutical compositions specific to LEREPO4 or GliPR.

Yet a further object of the present invention concerns a method of identifying inhibitors of LEREPO4 or at least one of its functional homologues which comprises the steps of
a) identifying physiological binding partners of LEREPO4 or at least one of its functional homologues;
b) providing at least one complex of LEREPO4 or at least one of its functional homologues and at least one of its physiological binding partners;
c) screening said at least one complex against compounds; and
d) identifying compounds that disrupt the interaction(s) between LEREPO4 or at least one of its functional homologues and at least one of its physiological binding partners within the complex.

Yet a further object of the present invention concerns a method of identifying inhibitors of GliPR or at least one of its functional homologues which comprises the steps of
a) identifying physiological binding partners of GliPR or at least one of its functional homologues;
b) providing at least one complex of GliPR or at least one of its functional homologues and at least one of its physiological binding partners;
c) screening said at least one complex against compounds; and
d) identifying compounds that disrupt the interaction(s) between GliPR or at least one of its functional homologues and at least one of its physiological binding partners within the complex.

### BRIEF DESCRIPTION OF THE FIGURES

FIG. 1 shows the DNA sequence (a, SEQ ID No. 3) and amino acid sequence (b, SEQ ID No. 4) of LEREPO4. The DNA sequence comprises up- and downstream regions. The start codon atg is underlined.

FIG. 2 shows the DNA sequence (a, SEQ ID No. 5) and amino acid sequence (b, SEQ ID No. 6) of GliPR. The DNA sequence comprises up- and downstream regions. The start codon atg is underlined.

FIG. 3 shows a schematic representation of an expression plasmid as it may be used for expression of shRNAs.

FIG. 4 shows further schematic representations of specific expression vectors for shRNAs such as pSHH, PEGFP-shRNA, pREV-shRNA and L1-shRNA.

FIG. 5 shows a sequence alignment of LEREPO4 and potential functional homologues of LEREPO4 (a). The numbers on the left are the GenBank accession numbers (http://www.ncbi.nlm.nih.gov/). The CCCH zinc finger motifes are indicated.

FIG. 6 shows a sequence alignment of GliPR and potential functional homologues of GliPR (a). The Genbank accession nos. of the homologues (http://www.ncbi.nlm.nih.gov/) are indicated on the left. The CRISP motifes are indicated.

FIG. 7 shows the results of TaqMan PCR analysis of P4-CCR5-cells after infection with HIV-1Bru at a MOI of 0.01 in comparison to a non-infected control. Errors bars indicate standard deviations as calculated from three independent experiments.

FIG. 8 (a) shows percentage change of expression of LEREPO4 and GliPR following infection with HIV-1Bru (MOI 0.01); FIG. 8 (b) shows the course of HIV infection in P4-CCR5-cells. HIV-RNA copy number was determined by TaqMan PCR and normalized against GAPDH (n=3).

FIG. 9 shows change of cellular gene expression for LEREPO4 as a function of HIV infection in Jurkat cells (Infection with HIV-1Bru, MOI of 0.01). Expression of LEREPO4 was determined using TaqMan PCR and normalized against GAPDH.

FIG. 10 shows efficiency of siRNA transfection in HeLa cells. A FACS Analysis was performed 18 h after transfection. Overlay of histograms shows a transfection efficiency of approximately 90% (a) for nonsilencing siRNA labelled with rhodamine (si-nons-Rho). HeLa cells were investigated for fluorescence after transfection with si-nons-Rho. Cell nucleus appears blue because of DAPI staining, siRNAs appear red (b).

FIG. 11 shows effect of transfection on proliferation in the WST-1 assay. Cells transfected with transfection reagent but without siRNAs were used as control compared to transfected (si-nons-Rho) and untransfected (unbehandelt) cells. WST-1 turn over of these cells was taken as 100% value. Error bars represent standard deviation as calculated from 5 experiments.

FIG. 12 shows the extent of gene supression mediated by LEREPO4- and GliPR-specific siRNAs. TaqMan analysis of transcription of (a) LEREPO4 or (b) GliPR) as determined for three different siRNAs indicated, respectively. Si-nons-Rho was used as a negative control in both panels. Transfection efficiency was approximately 90% and RNA was isolated 24 h after transfection. Error bars represent standard deviation calculated from 3 independent experiments.

FIG. 13 shows the reduction of LEREPO4 expression on the protein level. Total protein of transfected and control HeLa cells was harvested 48 h and 72 h after transfection. LEREPO4 expression was determined by western blotting. Tubulin detection served as an internal standard.

FIG. 14 shows the reduction of GliPR expression on the protein level. (a) HeLa cells were transfected with si-nons-Rho and transfection efficiency determined 24 h after transfection by FACS. (b) 24 h after co-transfection of si-nons-Rho and the expression plasmid pGLIPR-EGFP the transfection efficiency was determined. Compared to (a) transfection efficiency of siRNA transfected cells was reduced by approximately 20%. (c) Determination of EGFP expression 24 h after transfection of expression plasmid pGLIPR-EGFP (green) and co-transfection of pGLIPR-EGFP with si-nons-Rho (red). Co-transfection had no significant effect on the transfection of the expression plasmid. (d) Comparison of GLIPR-EGFP expression after co-transfection of GliPR-specific siRNA (blue) and si-nons-Rho (green). siGliPR transfection resulted in decreased GliPR expression.

FIG. 15 shows the effect of transfection of LEREPO4- and GliPR-specific siRNAs on cell proliferation in the WST-1 assay. Cells transfected with transfection agent but without siRNAs were used as control. WST-1 turn over of these cells was taken as 100% value. Error bars represent standard deviation as calculated from 5 experiments.

FIG. 16 shows the effect of repression of LEREPO4 and GliPR on HIV replication. Determination of the relative HIV-RNA copy number was done using real time TaqMan PCR after siRNA-induced repession of LEREPO4 and GliPR and subsequent HIV infection (HIV-1Bru, MOI of 0.01). As a control, cells were infected that had been transfected with si-nons-Rho. Cells which had been transfected with p24 specific siRNAs served as a positive control. The intracellular HIV-RNA copy numbers of days 0 to 12 after infection which have been normalized by GAPDH expression are shown. Error bars represent standard deviation of 3 experiments. In (a) representation on the y-axis is linear while a logarithmic scale is used in (b). The effect of si-GliPR is significantly stronger than the positive control with si-p24 inhibiting HIV directly. The inhibition by si-LEREPO4 shows also a significant effect compared to the negative control.

FIG. 17 shows the change of HIV-RNA copy number as percentage in reference to the control over time. The relative HIV-RNA copy number of si-nons-Rho transfected cells was taken as 0% change. The relative HIV-RNA copy numbers of si-LEREPO4 and si-GliPR transfected cells were then related to this control.

FIG. 18 shows the β-Galactosidase activity 4 days after infection of LEREPO4— and GliPR-specific siRNAs, si-nonsRho (negative control) and si-p24 (positive control) transfected cells. P4-CCR5 cells were infected with HIV-1Bru at a MOI of 0.01. The effect of repression of LEREPO4 and GliPR expression on HIV replication was measured using β-Galactosidase activity in these P4-CCR5-cells. The values were normalized using the WST-1-assay. Error bars represent standard deviation of 3 experiments.

FIG. 19 shows X-Gal staining of P4-CCR5 cells on day 7 after HIV infection. After transfection of LEREPO4- and GliPR-specific siRNAs, si-nonsRho (negative control) and si-p24 (positive control), cells were infected with HIV-1Bru at a MOI of 0.01. The reduced β-Galactosidase activity in the cultures with the LEREPO4— and GliPR-specific siRNAs compared to the negative control (si-nons-Rho) indicates the supression of HIV-1 replication.

FIG. 20 shows HIV p24 concentrations in cell culture supernatants as determined by ELISA. P4-CCR5-cells were transfected with LEREPO4- and GliPR-specific siRNAS, si-non-Rho (negative control) and si-p24 (positive control). Subsequently cells were infected with HIV-1Bru at an MOI of 0.01. p24 concentration was determined 0, 2 and 4 days after HIV infection (n=3).

FIG. 21 depicts a sequence in which the Z-finger domain of LEREPO4 is deleted.

FIG. 22 depicts putative dominant negative mutants of LEREPO4. Mutations are highlighted in red. X: represents a mutation from a conserved cystein into arginine, alanine, tyrosine or glutamate. Z: represents a mutation from histidine into aspartate, arginine or tyrosine.

FIG. 23 depicts putative dominant negative mutants of GLIPR. The CRISP family signatures are depicted in red fond. Mutations are highlighted in yellow. Z: represents a mutation from histidine into aspartate, arginine or tyrosine. ε: represents a mutation from glutamate into lysine or arginine.

FIG. 24 depicts the antisense sequence of LEREPO4 (SEQ ID No. 1).

FIG. 25 depicts the antisense sequence of GliPR (SEQ ID No. 2).

FIG. 26 depicts the amount of LEREPO4- and GliPR-specific mRNAs, 48 h after transfection of HeLa cells with the respective siRNA-construct. The mRNA-levels were quantified using Taqman realtime PCR. To calculate the relative expression levels, GAPDH mRNA levels were employed as a control. All measurements were performed in duplicate.

FIG. 27 shows the Co-Immunoprecipitation of LEREPO4 and TRAF2. LEREPO4-specific polyclonal antibodies were covalently linked to agarosebeads and columns were prepared. Total cell extracts from Jurkat cells were passed through the columns and after several washing steps eluates 1-4 (E1-4) were obtained by a shift in buffer-pH. LEREPO4 and TRAF2 co-eluted in E1 through E3 as was demonstrated by immunoblotting, using specific antibodies against LEREPO4 and TRAF2, respectively.

FIG. 28 shows the cytoplasmic localization of LEREPO4. HeLa cells were stained with LEREPO4-specific antibodies and Alexa-488-coupled secondary antibody. Nuclei were stained using DAPI.

FIG. 29 depicts induction of NF-κB activity by functional cooperation of LEREPO4 with TRAF2 and TRAF6. HeLa cells were transfected with different combinations of expression plasmids encoding for LEREPO4, TRAF2 and -6 (see below). NF-κB activity was measured using an ELISA-based method to detect binding of NF-κB to its consensus DNA-binding site.

FIG. 30 shows apoptosis-induction after transfection of cells with a GliPR-EGFP fusion protein. Depicted is the EGFP-fluorescence as a control of transfection (a, c), and apoptosis detection by Annexin V-staining and TUNEL-staining (b and d, respectively).

FIG. 31 shows apoptosis-induction in HeLa cells measured by TUNEL and Annexin V-staining (a). Also shown are characteristical changes in cell morphology, indicative of apoptotic cell death (b).

FIG. 32 depicts cell death elicited by expression of different GliPR-fusion proteins. The before-mentioned EGFP-fusion and a Strep-tag-fusion of GliPR induce apoptosis just as the GliPR open reading frame does.

FIG. 33 shows the location of a GliPR-EGFP fusion protein in the endoplasmic reticulum (ER). HeLa cells were transfected with GliPR-EGFP and stained with an antibody directed against proteindisulfide-isomerase (PDI), an ER-resident protein. Panel e) shows the co-localization of PDI and GliPR-EGFP in a fluorescence-overlay of the PDI-channel (red) and the EGFP-channel (green).

### DETAILED DESCRIPTION OF THE INVENTION

The present invention is based on the surprising finding that HIV infection of human cells leads to increased expression of the protein factors LEREPO4 and GliPR. These findings open the possibility of establishing methods of diagnosing HIV infections and/or occurrence of AIDS on the basis of increased LEREPO4 and/or GliPR expression in human cells.

Additionally, it was surprisingly found in the context of the present invention that down-regulation of LEREPO4's and/or GliPR's activity within human cells renders these cells less susceptible to HIV infection or less permissible for HIV replication.

This latter finding enables the design of inhibitor molecules, pharmaceutical compositions comprising these inhibitor molecules and methods of treatment which can be used to reduce, attenuate and/or prevent infection of cells with HIV, HIV replication in cells and/or development of AIDS.

It is also possible to use these findings to determine the susceptibility of a specific HIV isolate from a specific individual to the aforementioned methods of treating and preventing HIV infection/AIDS with said inhibitory molecules of pharmaceutical compositions specific to LEREPO4 or GliPR.

In addition, these findings open the possibility of assessing the prognosis or predicting the likelihood of therapeutic success to the aforementioned methods of treating and preventing HIV infection/AIDS with said inhibitory molecules of pharmaceutical compositions specific to LEREPO4 or GliPR by determining LEREPO4 or GliPR expression or by testing HIV isolates' strains' susceptibility to said therapy in vitro or in vivo.

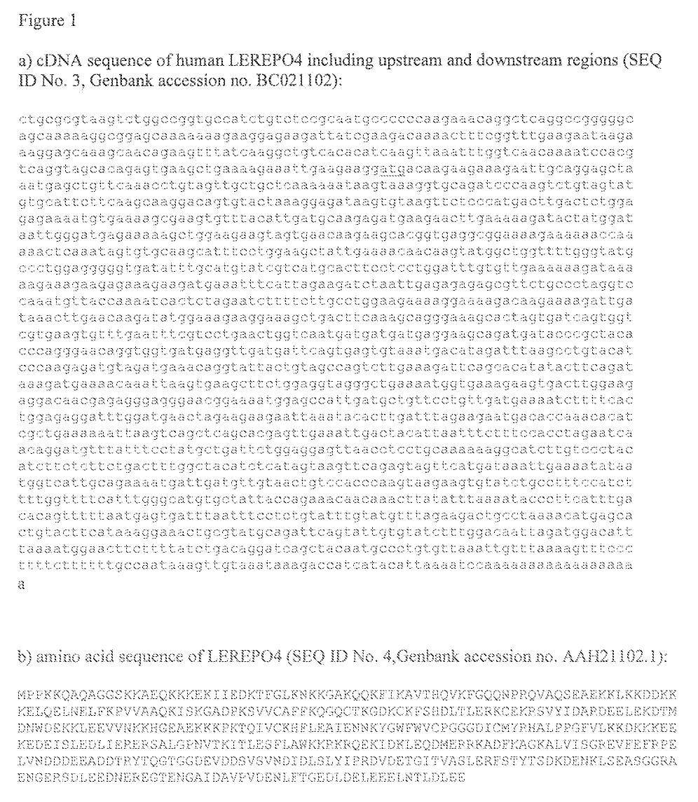

LEREPO4 is a protein found inter alia in human cells. The LEREPO4 protein may be encoded by a nucleic acid sequence molecule of SEQ ID No. 3 which depicts the coding sequence of LEREPO4 as found in the NCBI gene bank under Accession Code No. BC021102 (see also FIG. 1). The GeneID is: 55854. The LEREPO4 protein thus comprises the amino acid sequence of SEQ ID No. 4 which is also depicted in FIG. 1 and can be found in the NCBI gene bank under Accession No. AAH21102.1.

If in the context of the present invention reference is made to the function of LEREPO4, this designates the ability of LEREPO4 to influence HIV infectivity/replication of/in human cells. Thus, the function of LEREPO4 is considered to be measurable by down-regulating expression of LEREPO4 within cells and preferably human cells and measuring a decrease of HIV infectivity/replication. Experiment 3 below gives an example how different approaches can be used to determine whether repression of LEREPO4 leads to reduced HIV infectivity.

A functional homologue of LEREPO4 is defined by and may be identified using the following procedure. Typically, functional homologues of proteins are characterized by a high degree of sequence similarity, i.e. sequence identity. For the purposes of the present invention, a protein X (e.g. a LEREPO4 or GliPR homologue) is considered to be homologous to a protein Y (e.g. LEREPO4 or GliPR) if both proteins share a level of identity on the amino acid level of at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% over a contiguous stretch of at least 10, 12, 12, 13, 14, 15, 16, 17, 18, 19, 20, 21, 22, 23, 24, 25, 26, 27, 28, 29, 30, 50, 70, 100, 150, 200, 300 amino acids or their complete sequence lengths.

Homologies may be routinely determined using certain software programs, such as e.g. BLASTN, ScanProsite, the Laser gene software etc. Another possibility is to use the BLAST program package which can be found on the Internet homepage of the National Center for Biotechnology Information (http://www.ncbi.nlm.nih.gov/). Two proteins will also be considered to be homologous by way of sequence similarity if their respective encoding nucleic acid sequences provide the above mentioned identity grades.

If mentioned in the context of the present invention, BLAST searches will be performed using the program's default parameter settings at the filing date of this application.

However, while two proteins may e.g. share 90% homology, it may still be possible that they are not functional homologues, simply because one protein comprises e.g. an amino acid substitution in the amino acid sequence that leads to a loss of function of the protein.

For the purposes of the present invention, a protein will therefore only be considered to be a functional homologue of another protein if it shares the above specified degree of homology with the other protein and carries out essentially at least partially the same physiological function.

As a function of LEREPO4 is a decrease in HIV infectivity/replication, when LEREPO4's expression is suppressed, a functional homologue of LEREPO4 will thus be characterized by its sequence similarity to LEREPO4 and its capability of decreasing HIV infectivity/replication after repression of its expression.

Determining a decrease of HIV infectivity as measurable by decreased HIV replication as a consequence of the down-regulation of expression of LEREPO4 or one of its functional homologues may be carried out as described in Experiment 3.

Thus, if a nucleic acid or protein are suspected of encoding for or being a functional homologue of LEREPO4 because of their sequence homology to SEQ ID No. 3 and/or SEQ ID No. 4, expression of this protein may be down regulated e.g. by a method comprising the following steps:
designing an siRNA or antisenseRNA that specifically interferes with expression of a suspected functional homologue in mammalian cells, preferably human cells, most preferably HeLa (CD4+) cells;
infecting these cells with HIV; analyzing HIV replication
comparing these results with HIV replication rate in wild-type cells in which expression of the suspected homologue of LEREPO4 has not been interfered with.

Analysis of HIV replication may be performed using real-time PCR as described in Experiments 1 and 3; other methods may of course also be used, such as analysis of HIV replication by determining expression of a marker indicative of HIV replication such as described for a β-galactosidase expression system in Experiment 3.

Another possibility of determining HIV infectivity of mammalian cells is to determine the change in concentration of the HIV protein p24 as described in Experiment 3.

Further methods comprise e.g. the use of FACS analysis of other HIV proteins gp120, Tat etc., ELISA analysis of HIV proteins gp120, Tat etc., quantitative PCR of HIV-DNA or HIV-RNA (cDNA), tiration of virus copies.

Selection of suitable mammalian cell systems and infection thereof with HIV is well-known to the person skilled in the art. Suitable mammalian cell systems comprise e.g. P4-CCR5 cells, Hela-CD4 cells, T cells, monocytic/macrophage cells, astrocytic cells, GHOST cells, peripheral mononuclear cells etc.

Down-regulation of the expression of a putative functional homologue of LEREPO4 may be achieved as is described below for LEREPO4 itself. Thus, repression of expression of a functional LEREPO4 homologue may be achieved using antisense RNA strategies, siRNA inhibition, use of specific aptamers for the messenger RNA of the respective putative functional homologue of LEREPO4, homologous recombination using selective markers, etc.

If repression of the expression of putative functional LEREPO4 homologues is to be performed using antisense RNA or siRNA strategies, it is crucial to use an antisense or siRNA molecule that specifically interferes only with the expression of the putative functional homologue of LEREPO4 but not with the expression of another unrelated cellular factor. The selection, identification and production of such RNA-based inhibitors is well-known to the person skilled in the art and will be performed using the same line of reasoning that has been used when designing the siRNA inhibitors for expression of LEREPO4 as described in Example 2.

Typically, one will identify sequences within the coding sequence of the putative functional homologue of LEREPO4 that may be accessible for an antisense strategy or an siRNA-based approach in view of the absence of e.g. secondary structural elements which would likely be prohibitive for in vivo interaction between the inhibitory RNA-based molecules and the respective mRNA. Such stretches of nucleic acids may be identified using computer programs such as SFOLD.

In a second step such sequences will then be compared with other sequences in order to determine whether they are unique for the respective putative functional homologue of LEREPO4. This may be done be, e.g. performing BLAST searches at the National Center for Biotechnology Information (http://www.ncbi.nlm.nih.gov/).

Once one has identified unique sequences, antisense or siRNA inhibitors may be selected by using a complementary sequence that preferably exactly matches the identified sequences. Of course, lower degrees of complementarity may be used, provided that complementarity is not reduced to a level where interaction between antisense or siRNA-based inhibitors and messenger RNAs of other cellular factors than the putative functional homologue of LEREPO4 are likely to occur.

Typically, complementarity grades between the inhibitory RNA-based molecules and the identified specific sequences will be at least 90%, at least 91%, at least 92%, at least 93%, at least 94%, at least 95%, at least 96%, at least 97%, at least 98% or at least 99%.

Thus, if one has been able to show that down-regulation of expression of a putative functional homologue of LEREPO4 leads to reduced HIV replication in mammalian cell systems, and if the putative homologue of LEREPO4 provides the above-mentioned homology levels on the amino acid level, the putative homologue of LEREPO4 will be considered as a functional homologue of LEREPO4.

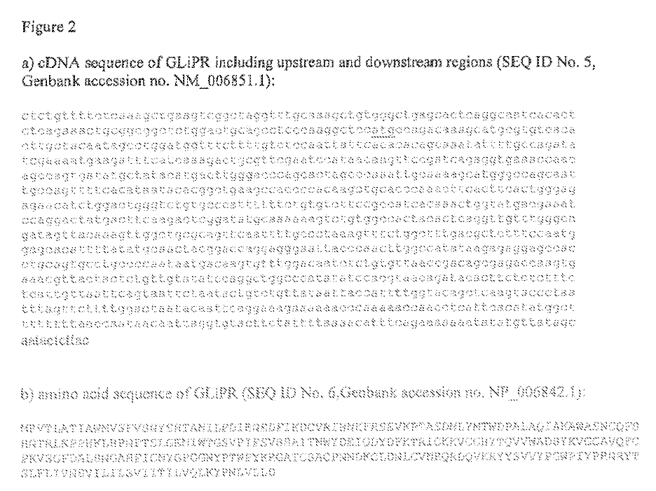

GliPR is a protein found inter alia in human cells. The GliPR protein may be encoded by a nucleic acid sequence molecule of SEQ ID No. 5 which depicts the coding sequence of GliPR as found in the NCBI gene bank under Accession Code No. NM_{—}006851.1 (see also FIG. 2). The GeneID is: 11010. The GliPR protein thus comprises the amino acid sequence of SEQ ID No. 6 which is also depicted in FIG. 2 and can be found in the NCBI gene bank under Accession No. NP_{—}006842.1.

If in the context of the present invention reference is made to the function of GliPR, this designates the ability of GliPR (as is the case for LEREPO4) to influence HIV infectivity of human cells. Thus, the function of GliPR is considered to be measurable by down-regulating expression of GliPR within cells and preferably human cells and measuring a decrease of HIV infectivity/replication. Experiment 3 gives an example how different approaches can be used to determine whether repression of GliPR leads to reduced HIV infectivity.

Functional homologues of GliPR are defined as for LEREPO4. They thus have to provide the above cited sequence identity to GliPR and provide the same function as GliPR. The function of GliPR and its functional homologues is measured using the same approach as laid out for LEREPO4.

As set out above, one object of the present invention relates to inhibitor molecules which are capable of interfering with the function of LEREPO4 or functional homologues thereof, meaning that these inhibitor molecules are able to down-regulate the expression of LEREPO4 or its functional homologues, thereby inducing a reduced infectivity of the respective cells for HIV, measurable by a reduced replication of the virus within a cellular system.

One class of such inhibitor molecules may be molecules comprising a recombinant nucleic acid molecule having a nucleic acid sequence which is complementary (and/or specific) to the complete coding sequences or parts thereof of LEREPO4 or its functional homologues. It is to be noticed that a definition of LEREPO4 and the meaning of the term “functional homologue” has been specified above. It is further to be noticed that in the context of these recombinant nucleic acid molecules, the term “complementary” or “complementarity” is to be understood as indicating that a nucleic acid sequence within the recombinant nucleic acid molecule which forms part of or is identical with the inhibitor molecule is able to specifically interact with the coding sequence or parts of LEREPO4 or its functional homologues by way of base complementarity. This, of course, applies mutatis mutandis in the context of GliPR.

The term “complementarity” is of course well-known to the person skilled in the art as it relates to the basic properties of nucleic acid molecules hybridizing with each other in view of the ability of adenosine to pair with thymine and uracil and guanidine to pair with cytidine.

If, in the context of the referenced inhibitory molecule, the nucleic acid sequence is said to be complementary to the coding sequence of e.g. LEREPO4, this does not mean that the nucleic acid sequence has to be 100% complementary, i.e. there is no requirement that the coding sequence of LEREPO4 and the reference inhibitory molecule have a 100% match. Rather, the term “complementary” means that the inhibitory molecule comprising the recombinant nucleic acid molecule in view of the complementary nucleic acid sequence is capable of hybridizing under in vivo conditions specifically with the complete coding sequence or parts thereof of LEREPO4 or its functional homologues. In a preferred embodiment, the inhibitory molecules comprise a recombinant nucleic acid molecule with a complementary nucleic acid sequence that is capable of hybridizing under stringent in vitro conditions with the complete coding sequence or parts thereof of LEREPO4 or functional homologues thereof.

According to the present invention, hybridization is carried out in vivo or in vitro under conditions that are stringent enough to ensure a specific hybridization.

Stringent in vitro hybridization conditions are known to the person skilled in the art and can be taken from the literature (see e.g. Sambrook et al. A Laboratory Manual, Cold Spring Harbour Laboratory Press, 2001).

The term “specific hybridization” refers to the case wherein a molecule preferentially binds to a certain nucleic acid sequence under stringent conditions, if this nucleic acid sequence is part of a complex mixture of e.g. DNA or RNA molecules.

The term “stringent conditions” therefore refers to conditions, under which a nucleic acid sequence preferentially binds to a target sequence, but not, or at least to a significantly reduced extent, to other sequences.

Stringent conditions are dependent on the circumstances. Longer sequences specifically hybridize at higher temperatures. In general, stringent conditions are chosen in such a way that the hybridization temperature lies about 5° C. below the melting point (Tm) of the specific sequence with a defined ionic strength and a defined pH value. Tm is the temperature (with a defined pH value, a defined ionic strength and a defined nucleic acid concentration), at which 50% of the molecules, which are complementary to a target sequence, hybridize with said target sequence. Typically, stringent conditions comprise salt concentrations between 0.01 and 1.0 M sodium ions (or ions of another salt) and a pH value between 7.0 and 8.3. The temperature is at least 30° C. for short molecules (e.g. for such molecules comprising between 10 and 50 nucleotides). In addition, stringent conditions can comprise the addition of destabilizing agents like e.g. formamide. Typical hybridization and washing buffers are of the following composition.

| | |
|---|---|
| Pre-hybridization solution: | 0.5% SDS |
| | 5x SSC |
| | 50 mM NaPO₄, pH 6.8 |
| | 0.1% Na-pyrophosphate |
| | 5x Denhardt's reagent |
| | 100 μg/salmon sperm |
| Hybridization solution: | Pre-hybridization solution |
| | 1 × 10⁶ cpm/ml probe (5-10 min 95° C.) |
| 20x SSC: | 3 M NaCl |
| | 0.3 M sodium citrate |
| | ad pH 7 with HCl |
| 50x Denhardt's reagent: | 5 g Ficoll |
| | 5 g polyvinylpyrrolidone |
| | 5 g Bovine Serum Albumin |
| | ad 500 ml A. dest. |

A typical procedure for the hybridization is as follows:

| Optional: | wash Blot 30 min in 1x SSC/0.1% SDS at 65° C. | | |
|---|---|---|---|
| Pre-hybridization: | at least 2 h at 50-55° C. | | |
| Hybridization: | over night at 55-60° C. | | |
| Washing: | 05 min | 2x SSC/0.1% SDS | |
| | Hybridization temperature | | |
| | 30 min | 2x SSC/0.1% SDS | |
| | Hybridization temperature | | |
| | 30 min | 1x SSC/0.1% SDS | |
| | Hybridization temperature | | |
| | 45 min | 0.2x SSC/0.1% SDS | 65° C. |
| | 5 min | 0.1x SSC | room temperature |

It is furthermore to be noted that the inhibitory molecules comprising a recombinant nucleic acid molecule comprising a complementary nucleic acid sequence may not only be complementary to the coding sequence or parts thereof LEREPO4 or its functional homologues, but also to the complete mRNA or parts thereof of GliPR or functional homologues thereof.

The person skilled in the art will, of course, understand that the nucleic acid sequence conferring complementarity with the complete coding sequence or parts thereof of LEREPO4 or its functional homologues must have a certain minimum length in order to ensure that the complementary sequence is indeed specific for LEREPO4 or its functional homologues.

Therefore, the nucleic acid sequence within the inhibitory molecule which confers complementarity to LEREPO4 or its functional homologues should be at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 50, at least 75, at least 100, at least 150, at least 200, at least 300 or at least 400 nucleotides in length and forming a contiguous stretch.

The person skilled in the art will of course also be aware that depending on the length of the nucleic acid sequence conferring complementarity to the coding sequences or parts thereof of LEREPO4 or its functional homologues, the minimum degree of complementarity which is necessary to ensure that the complementarity-conferring nucleic acid sequence indeed only specifically targets LEREPO4 or its functional homologues will vary. Thus, if a rather long nucleic acid molecule of 30, 50, 60, 75, 100 nucleotides is used, a lower degree of complementarity may be used and still ensure specificity between the inhibitory molecule and the coding sequence or parts thereof of LEREPO4 or its functional homologues, while of course a higher degree of complementarity will be necessary if the complementarity-conferring nucleic acid sequence within the inhibitory molecule comprises e.g. only a stretch of at least 14, 15, 16, 17, or 19 nucleotides.

Similarly, as in the case of homology (mentioned/described) above, complementarity may be defined or described in terms of percentages. 100% complementarity thus refers to a situation where two nucleic acid molecules are able to hybridize over the entire length of both molecules without any mismatch under stringent conditions as set out above. Lower degrees of complementarity will relate to situations where the two nucleic acid molecules may still hybridize under stringent conditions, even though some mismatches may occur. If a nucleic acid sequence X and a nucleic acid sequence Y are found to be 90% homologous, this, as described above for amino acid sequences, refers to a situation where both molecules over their entire length share 90% identical nucleotides if both (are) aligned in 5′-3′ direction. As for proteins, such homology comparisons between nucleic acid sequences may be performed using the aforementioned software programs.

Accordingly, if sequence X is found to be 50% homologous to sequence Y, this also means that sequence X will share 50% complementarity with the sequence that is 100% complementary to sequence Y. The interrelation between homology and complementarity of two nucleotide sequences and their 100% complements is a direct result of the above-described specific interaction between nucleotide bases.

As a consequence, for the purposes of the present invention two nucleotide sequences are considered to be complementary if they are able to hybridize under stringent conditions and have a complementarity of at least 30%, at least 35%, at least 40%, at least 45%, at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% with a target sequence such as SEQ ID No. 3 in the case of LEREPO4 or SEQ ID No. 5 in the case of GliPR over a contiguous stretch of at least 10, 11, 12, 13, 14, 15, 16, 17, 18, 19, 20, 21, 22, 23, 24, 25, 26, 27, 28, 29, 30, 50, 70, 100, 150, 200, 300 or more nucleotides.

The above-described inhibitory molecules of LEREPO4 function which comprise a recombinant nucleic acid molecule comprising a nucleic acid sequence being complementary to the complete coding sequence or parts thereof of LEREPO4 or functional homologues thereof, may preferably comprise SEQ ID No. 1 or parts thereof. In a preferred embodiment, the nucleic acid sequence may show a degree of identity of at least 30%, at least 35%, at least 40%, at least 45%, at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% with SEQ ID No. 1 over a contiguous stretch of at least 10, 11, 12, 13, 14, 15, 16, 17, 18, 19, 20, 21, 22, 23, 24, 25, 26, 27, 28, 29, 30, 50, 70, 100, 150, 200, 300 or more nucleotides.

In a preferred embodiment, the present invention relates to recombinant nucleic acid molecules comprising SEQ ID Nos. 14, 15 or 16 if repression of LEREPO4 is envisaged.

As already indicated, another object of the present invention relates to inhibitor molecules which are capable of interfering with the function of GliPR or functional homologues thereof, meaning that these inhibitor molecules are able to down-regulate the expression of GliPR or its functional homologues, thereby inducing a reduced infectivity of the respective cells for HIV, measurable by a reduced replication of the virus within a cellular system.

One class of such inhibitor molecules may therefore be molecules comprising a recombinant nucleic acid molecule having a nucleic acid sequence which is complementary to the complete coding sequences or parts thereof of GliPR or its functional homologues. The above explanations in the context of LEREPO4 as to the required complementarity grade, stringent hybridization conditions equally apply to the case of GliPR.

The inhibitory molecules of GliPR function which comprise a recombinant nucleic acid molecule comprising a nucleic acid sequence being complementary to the complete coding sequence or parts thereof of GliPR or functional homologues thereof, may preferably comprise SEQ ID No. 2 or parts thereof. In a preferred embodiment, the nucleic acid sequence may show a degree of identity of at least 30%, at least 35%, at least 40%, at least 45%, at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% with SEQ ID No. 2 over a contiguous stretch of at least 10, 11, 12, 13, 14, 15, 16, 17, 18, 19, 20, 21, 22, 23, 24, 25, 26, 27, 28, 29, 30, 50, 70, 100, 150, 200, 300 or more nucleotides.

In a preferred embodiment, the present invention relates to recombinant nucleic acid molecules comprising SEQ ID Nos. 17, 18 or 19 if repression of GliPR is envisaged.

In both cases, LEREPO4 or GliPR, the nucleic acid sequence molecules may be made of DNA, RNA or other nucleic acid based molecule, which, however, instead of nucleotides may comprise at least partially nucleotide analogues as long as the resulting molecule is capable of specifically hybridizing with the complete coding sequence or parts thereof of LEREPO4 or GliPR and their respective functional homologues.

In a preferred embodiment, the above described inhibitory molecules preferably comprise antisense RNA, shRNA, siRNA, miRNA or other nucleic acid molecules of comparable function to LEREPO4 or GliPR or their respective functional homologues.

These terms clearly indicate to the person skilled in the art how these molecules should be synthesized, what degree of complementarity should be considered, etc. It is to be understood that antisense RNAs as well shRNAs and siRNAs in accordance with the invention will fulfill the above mentioned criteria as to the length of these nucleic acid molecules, their degree of complementarity etc. with SEQ ID No. 3 (LEREPO4) or SEQ ID No. 5 (GliPR) or parts of these sequences as well as functional homologues thereof

An antisense molecule will typically comprise a length of 10, 11, 12, 13, 14, 15, 16, 17, 18, 19, 20, 21, 22, 23, 24, 25, 26, 27, 28, 29, 30, 50, 70, 100, 150, 200, 300 or more nucleotides.

For the purposes of RNA interference so-called short hairpin RNA or synthetic double-stranded siRNA oligonucleotides may be used.

An siRNA molecule will typically comprise a length of 20, 21, 22, 23, 24 or 25 nucleotides

Typically, as shRNA molecules are expressed, single-stranded RNA molecules that form an intramolecular hairpin structure. By intracellular processing through the enzyme Dicer, siRNA molecules are generated therefrom.

An intracellular transcription of shRNA molecules can be achieved if both strands of an shRNA duplex are expressed using an expression vector in the form of a single RNA molecule or DNA molecule. For this purpose, the transcribed RNA strand should ideally comprise 19 to 21 nucleotides of the sense shRNA sequence and ideally 19 to 21 nucleotides of the corresponding complementary sequence. Both sequences would ideally be separated e.g. by a six nucleotide long spacer.

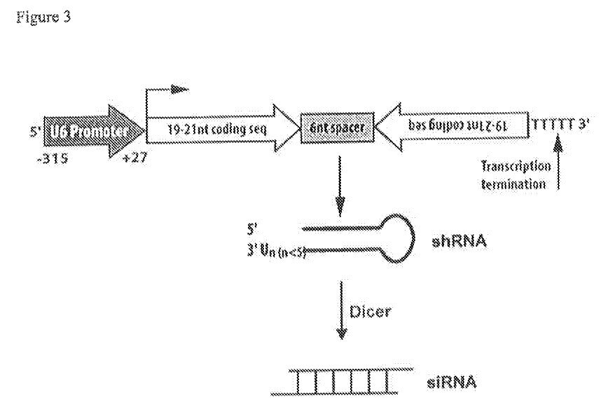

An expression vector which, upon introduction into a host cell ensures expression and transcription of such RNA molecules is indicated in FIG. 3. In an embodiment, transcription of the shRNA occurs from a U6 promoter belonging to the class of Pol III promoters. The termination signal may be defined by a sequence of five thymidines.

It is well known that efficiency of siRNA mediated repression of cellular target transcripts is dependent on the choice of suitable siRNA sequences. Guidelines have been developed for the design of effective siRNA molecules. These guidelines have been typically derived for synthetic siRNA oligonucleotides, but should also apply for the processed form of shRNA molecules. Examples are given below as to how such guidelines may be used for the design of effective shRNA molecules or synthetic siRNA molecules.

Other than shRNA molecules which are only obtained after expression and processing within the cells, synthetic siRNA oligonucleotides consist of double-stranded RNA molecules which are typically between 19 to 21 nucleotides long. These siRNA molecules may e.g. be transfected into cellular systems and will thus initiate a RNAi process.

Determination of the targeted sequence and siRNA sequence motif may e.g. be determined according to well known publications such as those by Tuschl et al. Thus, one may use the coding region of a target mRNA for identification of suitable siRNA target sequences only. While it is preferred that the selected siRNA sequence motif is directed to the coding region of the target mRNA, it may also be designed against the regulatory regions of a gene such as the 5′ and 3′ untranslated regions of the LEREPO4 or GliPR genes.

If coding sequences of a messenger RNA are used as target sequences, one may typically use sequences starting 70 nucleotides downstream of the start codon and ending 50 nucleotides upstream of the stop codon.

This sequence area may then be searched for the sequence motive AA (N19) in which N designates any nucleotide. The resulting siRNA sequence will then comprise 19 nucleotides following the motive AA and preferably two additionally added uridine or thymidine residues. In the case of synthetic siRNA oligonucleotides, the uridine residues may preferably be replaced by thymidine.

In another approach, guidelines may be used according to Reynolds et al.

Reynolds et al. have suggested the following criteria for selecting potential shRNA or synthetic siRNA target sequences:
1. the 30-50% guanine-cytosine content
2. at least three adenines or uracils at positions 15 to 19 of the sense strand
3. absence of intermolecular hairpin structures
4. adenine at position 19 of the sense strand
5. adenine at position 3 of the sense strand
6. uracil at position 10 of the sense strand
7. no guanine or cytosine at position 19 of the sense strand
8. no guanine at position 13 of the sense strand.

These eight criteria may be weighed according to the following scheme:
(i) 1 point for criteria 1, 3, 4, 5 and 6
(ii) 1 point for each adenine or uridine at position 15 to 19, at least 3 corresponding bases (criterion 2)
(iii) non-fulfillment of criteria 7-8 result in -1 point each.

According to Reynolds, only siRNA or shRNA sequences should be considered which according to this scheme have a point value of at least 6. Such siRNA sequences may then be used for a homology search using the BLAST program. Following this approach, siRNAs may be excluded that as a matter of their homology with other coding mRNA sequences would lead to non-specific repression of target structures.

If siRNA or shRNA sequences have been identified this way, they may be cloned into an expression plasmid. Thus, the RNA sequences may be cloned into the plasmid pSuppressor (pSHH, Imgenex, San Diego, Calif., USA). For cloning the siRNA sequences into the pSHH constructs, hybridised DNA oligonucleotides can be used that comprise the siRNA sense sequence, a spacer, the corresponding antisense sequence and the termination sequence.

In order to have a detectable or selectable marker gene, GFP may be used to monitor the shRNA expression plasmid for determining transfection efficiency using FACS. A further strategy would be the construction of retroviral shRNA expression vectors in order to transduce hardly transfectable cells such as e.g. T-cells or primary cells with an increased efficiency. As a starting vector one may use plasmids such as pCMS-EGFP (Clontech) or the retroviral vectors pRevTRE (Clontech) and L1. In case of plasmids pCMS-EGFP and pRevTRE, CMV-promoters may be removed by restriction digest and replaced with the shRNA expression cassette from plasmid pSHH.

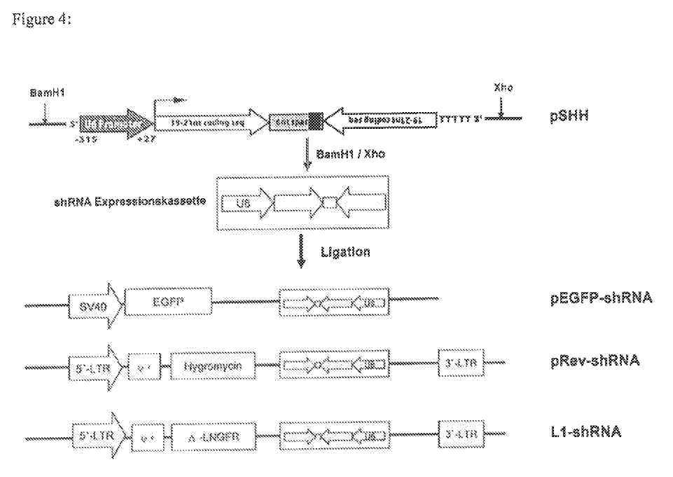

The shRNA expression cassette may also be cloned directly into plasmid L1, as this vector only comprises the 5′-LTR as eukaryotic promoter. The restriction sites which are necessary for cloning may be introduced into the plasmids in advance using suitable DNA linkers. The resulting plasmids may be designated as pEGFP-shRNA, pRev-shRNA and L1-shRNA. They are schematically depicted in FIG. 4.

Use of the plasmid pEGFP-shRNA allows determination of transfection efficiency using FACS analysis. For example, the retroviral vector pRev-shRNA carries the selection marker hygromycine. It also has the advantage that cells which are difficult to transfect can be transduced with great efficiency. The second retroviral vector L-shRNA comprises the selection marker of a truncated version of the low affinity nerve growth factor receptor (ΔLNGF) under the control of the 5′-LTR. This truncated version possesses a shortened cytoplasmic domain and thus cannot contribute to signal transduction. Using this vector has the advantage that it allows determination of transduction efficiency using FACS analysis by determining ΔLNGF expression.

If synthetic siRNA oligonucleotides are used, these may be transferred into cells using techniques well known to the person skilled in the art, such as typical transfection protocols.

Besides lipofection, further approaches for transducing synthetic siRNAs into a host cell comprise injection procedures, electroporation, protein transduction and other techniques.

Yet another embodiment of the invention relates to recombinant nucleic acid molecules in which an antisense sequence as described above of e.g. at least 10, 11, 12, 13, 14, 15, 17, 18, 19, 20, 25, 30 or 35 nucleotide length is fused to a Ribonuclease P sequence. This type of molecule will be directed by means of the antisense sequence in vivo e.g. to the mRNA of LEREPO4 or GliPR. After targeting, Ribonuclease P will destroy the mRNA.

In another approach, RNA based aptamers may be developed which specifically recognise the mRNA of LEREPO4 or GliPR. Depending on whether the aptamer has ribonuclease activity or not, it may not only specifically target the aforementioned mRNAs but also destroy them. Of course, such aptamers can also be coupled to Ribonuclease P. The expression of aptamers is usually achieved by vector-based overexpression and is, as well as the design and the selection of aptamers, well known to the person skilled in the art (Famulok et al., (1999) Curr Top Microbiol Immunol., 243, 123-36).

In one embodiment the invention relates to nucleic acid molecules which encode any of the aforementioned nucleic acid molecules. Thus this embodiment relates e.g. to a vector which upon transcription gives rise to the aforementioned antisense RNAs, siRNAs, shRNAs, ribozymes, aptamers and fusion constructs in vivo.

These vectors can among others include virus based vectors as they are used for gene therapy approaches. A typical embodiment of these latter inhibitory molecules are e.g. virus-based vectors that can e.g. be used to transfect cellular systems or individuals in order to ensure an in vivo expression of an antisense RNA or an siRNA in the cells of e.g. a human individual for silencing endogeneous LEREPO4 or GliPR expression or expression of their respective functional homologues. There is a large variety of viral vectors that have been investigated preclinically, some of which have already been employed in clinical studies. The most important groups of viral vectors are based on RNA viruses, especially lentiviruses and in particular retroviruses, and DNA viruses, especially adeno viruses, adeno-associated viruses, poxviruses and others [Verma I M, Weitzman M D Annu Rev Biochem 2005; 74:711-38]. Such vectors can be used e.g. in a gene therapy approach to downregulate expression of LEREPO4 and/or GliPR and to thereby interfere with HIV replication.

In one embodiment the invention relates to a recombinant nucleic acid molecule comprising an expression vector for transfection of a mammalian cell comprising at least one of the afore mentioned nucleic acid molecules, i.e. antisense RNA, siRNAs, shRNA, ribozymes, aptamers etc. and regulatory sequences operatively linked to these latter nucleic acid molecules to allow transcription of said nucleic acid molecules in said cell.

In one embodiment the invention thus relates to a recombinant nucleic acid molecule, wherein said recombinant nucleic acid molecule is a vector comprising:
a promoter being functional in mammalian cells,
operatively linked thereto a nucleic acid sequence coding at least for an antisense RNA to the complete coding sequence or parts thereof of LEREPO4 or GliPR or their respective functional homologues, and
a termination sequence.

In another embodiment the invention thus relates to a recombinant nucleic acid molecule, wherein said recombinant nucleic acid molecule is a vector comprising:
a promoter being functional in mammalian cells,
operatively linked thereto a nucleic acid sequence coding at least for a first nucleic acid sequence being complementary to the complete coding sequence or parts thereof of LEREPO4 or GliPR or their respective functional homologues, optionally a spacer nucleic acid sequence and a second nucleic acid sequence being complementary to said first nucleic acid sequence, and
a termination sequence.

This latter embodiment thus relates to a vector encoding for shRNA inhibitors of LEREPO4 or GliPR function. The sequences of the shRNA molecules may be determined as described above and embodied for siRNA inhibitors in Examples 2 and 3. Thus one may first design an siRNA molecule and test its effects in e.g. a cell culture system and then use the identified suitable sequence for the above vector.

Yet another embodiment of the invention thus relates to a recombinant nucleic acid molecule, wherein said recombinant nucleic acid molecule is a vector comprising:
a promoter being functional in mammalian cells,
operatively linked thereto a nucleic acid sequence coding at least for a nucleic acid sequence being complementary and/or specific to the complete coding sequence or parts thereof of LEREPO4 or GliPR or their respective functional homologues,
a nucleic acid sequence encoding ribonuclease P, and
a termination sequence.

In another embodiment, the invention relates to recombinant nucleic acid molecules such as vectors that can be used to disrupt the function of endogenous LEREPO4 or GliPR by interfering with the function of these genes by homologous recombination. Thus, this embodiment of the invention relates so called “knock outs” of the genes encoding LEREPO4 or GliPR or their respective functional homologues. Such vectors can be used in gene therapy approaches to permanently down regulate expression of LEREPO4 or GliPR or their respective functional homologues and thus to render cells and individuals less susceptible to HIV infections and AIDS. These vectors may also be used to manipulate cells such as pluripotent stem cells, hematopoietic stem cells, bone marrow derived hematopietic precursor cells, peripheral mononuclear cells, lymphocytes, monocytes, dendritic cells, astrocytes (incl. precursors) and all other potential target and bystander cells for HIV infection, which have been retrieved from HIV positive individuals or individuals at risk for HIV infection, but which have not yet been infected by the virus. These cells can then be manipulated in vitro by homologous recombination to shut of expression of LEREPO4 or GliPR or their respective functional homologues. Thereafter these cells are expanded in vitro and reintroduced into the individual. This autologous replacement therapy is one way of attenuating propagation of the virus.

Thus, one embodiment of the invention relates to a recombinant nucleic acid molecule,

wherein said recombinant nucleic acid molecule is a vector comprising:
a promoter being functional in mammalian cells,
a nucleic sequence being identical or homologous to the sequence encoding the 5′ end of LEREPO4 or GliPR or their respective functional homologues;
a nucleic acid sequence encoding a selectable marker;
a nucleic sequence being identical or homologous to the sequence encoding the 3′ end of LEREPO4 or GliPR or their respective functional homologues; and
a termination sequence.

The commonly used selectable markers are known to the person skilled in the art and selecting a suitable marker does not pose a problem. Common selection markers are such, which confer resistance against a biocide or an antibiotic like kanamycin, G418, bleomycin, hygromycin, methotrexate, glyphosate, streptomycin, sulfonyl urea, gentamycin or phosphinotricin.

A particularly preferred embodiment of the present invention relates to the use of the aforementioned inhibitory molecules for inhibiting the function of LEREPO4 or its functional homologues in cells. These cells are preferably of mammalian origin and particularly preferably are of human origin. In a preferred embodiment these cells form a part of the human or animal body including but not limited to the immune system, bone marrow, blood, central nervous system etc. In particular pluripotent stem cells, hematopoietic stem cells, bone marrow derived hematopoietic precursor cells, peripheral mononuclear cells, lymphocytes, monocytes, dendritic cells and all other potential target and bystander cells for HIV infection are included.

Yet another particularly preferred embodiment of the present invention relates to the use of the aforementioned inhibitory molecules for inhibiting the function of GliPR or its functional homologues in cells. These cells are again preferably of mammalian origin and particularly preferably are of human origin. In a preferred embodiment these cells form a part of the human or animal body including but not limited to the immune system, bone marrow, blood, central nervous system etc. In particular pluripotent stem cells, hematopoietic stem cells, bone marrow derived hematopoietic precursor cells, peripheral mononuclear cells, lymphocytes, monocytes, dendritic cells and all other potential target and bystander cells for HIV infection are included.

Another embodiment of the present invention relates to a molecule comprising a recombinant nucleic acid molecule encoding a dominant-negative proteinaceous mutant of LEREPO4 or functional homologues thereof.

One embodiment of the present invention also relates to a molecule comprising a recombinant nucleic acid molecule encoding a dominant-negative proteinaceous mutant of GliPR or functional homologues thereof.

The term “dominant-negative proteinaceous mutant” refers to a protein which differs from e.g. SEQ ID No. 4 or SEQ ID No. 6 in that it comprises amino acid substitutions, deletions or insertions which lead to a loss of function of wild-type LEREPO4 (in case of SEQ ID No. 4) or GliPR (in case of SEQ ID No. 6), if wild-type LEREPO4 or wild type GliPR respectively and the respective dominant-negative proteinaceous mutant are simultaneously present in a ratio of at least 1:1, 1:2, 1:3, 1:4, etc.

Thus, a dominant-negative proteinaceous mutant of LEREPO4 or GliPR or accordingly of one of their respective functional homologues is capable of cross-competing with wild-type LEREPO4 or wild-type GliPR or their respective wild-type functional homologues for interaction with the respective physiological cellular binding partners.

As a consequence of this cross-competition for binding, a reduced amount of functional wild-type LEREPO4 or GliPR is available for the interactions within the cell, leading to an effect similar to a reduced expression of LEREPO4 or GliPR or of their respective functional homologues.

A further consequence of this cross-competition of the interaction between LEREPO4 or its functional homologues (and the same in the case of GliPR) and their physiological endogenous or viral binding partners is a reduced infectivity of these cells by HIV as measurable in the form of reduced HIV replication.

Dominant-negative mutants of LEREPO4 or GliPR may be identified by a process well known to the person skilled in the art. One may thus carry out homology searches in order to identify domains, amino acid regions or conserved amino acid positions within the amino acid sequence of LEREPO4 or GliPR and their respective functional homologues within one single organism or between different organisms. As such conserved parts of the LEREPO4 or GliPR amino acid sequences are likely to be essential for the proper functioning of LEREPO4 or GliPR in vivo, deleting (parts of) such regions or amending conserved amino acid positions within such regions may lead to versions of LEREPO4 or GliPR that may no longer be able to function properly and thus constitute bona fide candidates for dominant-negative mutants.

By introducing amino acid substitutions, inserting additional amino acids or deleting amino acids in these conserved regions, one can obtain mutants of LEREPO4 and its functional homologues that are likely to interfere with the function of wild-type LEREPO4 or of wild-type functional homologues. The same applies again in the case of GliPR and its functional homologues.

If, for example, a search for conserved protein motifes is performed in the databases PROSITE [Hulo N et al. The PROSITE database; Nucleic Acids Res (2006), 34:D227-D230; Sigrist C J A et al. PROSITE: a documented database using patterns and profiles as motif descriptors. Brief Bioinform (2002), 3:265-274] and PFAM [Bateman A et al. Nucleic Acids Research, (2004) Database Issue 32:D138-D141] under standard parameters, it can be shown that LEREPO4 comprises two putative zinc finger motifs of the CCCH type. Zinc finger motives of the CCCH type are commonly found in proteins involved in protein-protein and protein-nucleic acid interactions. They typically constitute a DNA-binding or RNA-binding domain.

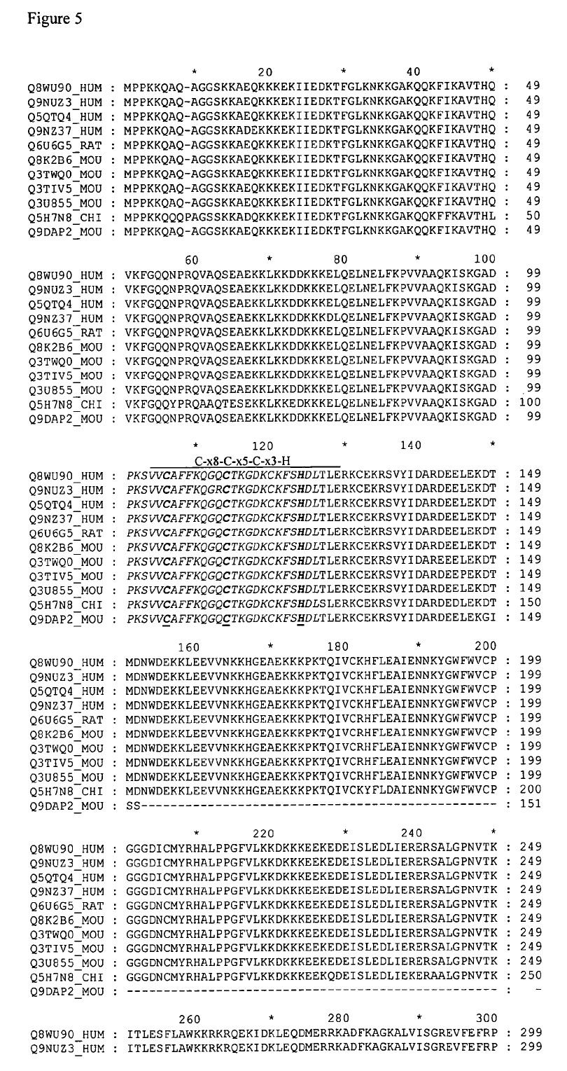

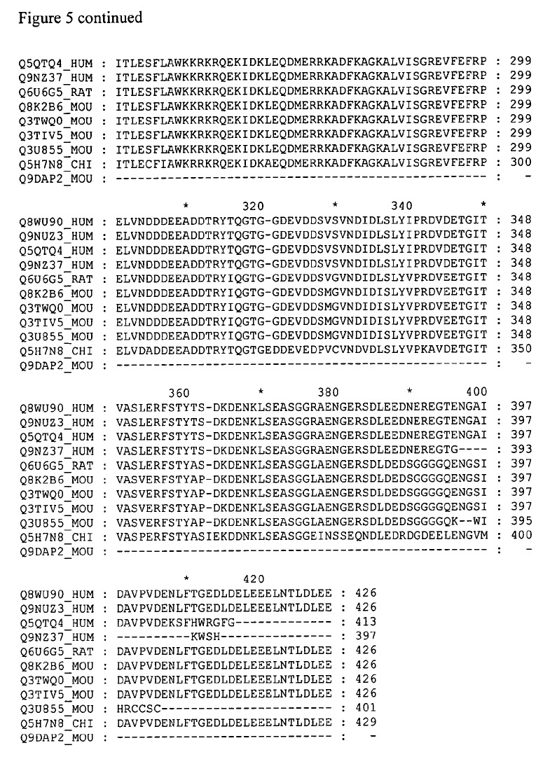

As an example, FIG. 5 shows the sequence alignment of LEREPO4 and its putative functional homologues. As can be deduced from FIG. 5, the CCCH type motive is clearly conserved. There are other domains that are conserved among the homologous sequences in FIG. 5, in particular amino acids (aa) 18-43, 79-100, 127-142, 151-181 with amino acid numbering being referenced to SEQ ID No. 4.

In the case of human LEREPO4, the zinc finger motif is thus found at amino acid positions 100-125 of SEQ ID No. 4.

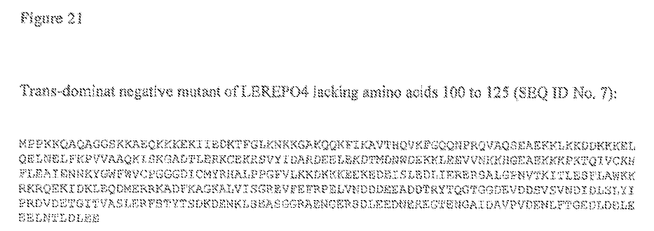

If a recombinant version of human LEREPO4 is produced that lacks the zinc finger motif at this position or comprises non-conserved amino acid substitutions within this conserved region, the resulting protein is likely to be a dominant-negative mutant of LEREPO4. Thus SEQ ID No. 7 in which amino acids 100 to 125 are deleted is a putative trans-dominant mutant of LEREPO4 (see also FIG. 21).

Similarly, point mutants of LEREPO4 which encode for an Arginine, Alanin or Cysteine at position 105 of SEQ ID No. 4 instead of Cysteine are putative dominant negative mutants of LEREPO4.

Point mutants of LEREPO4 which encode for an Aspartate or Glutamate at position 123 of SEQ ID No. 4 instead of Cysteine are putative dominant negative mutants of LEREPO4.

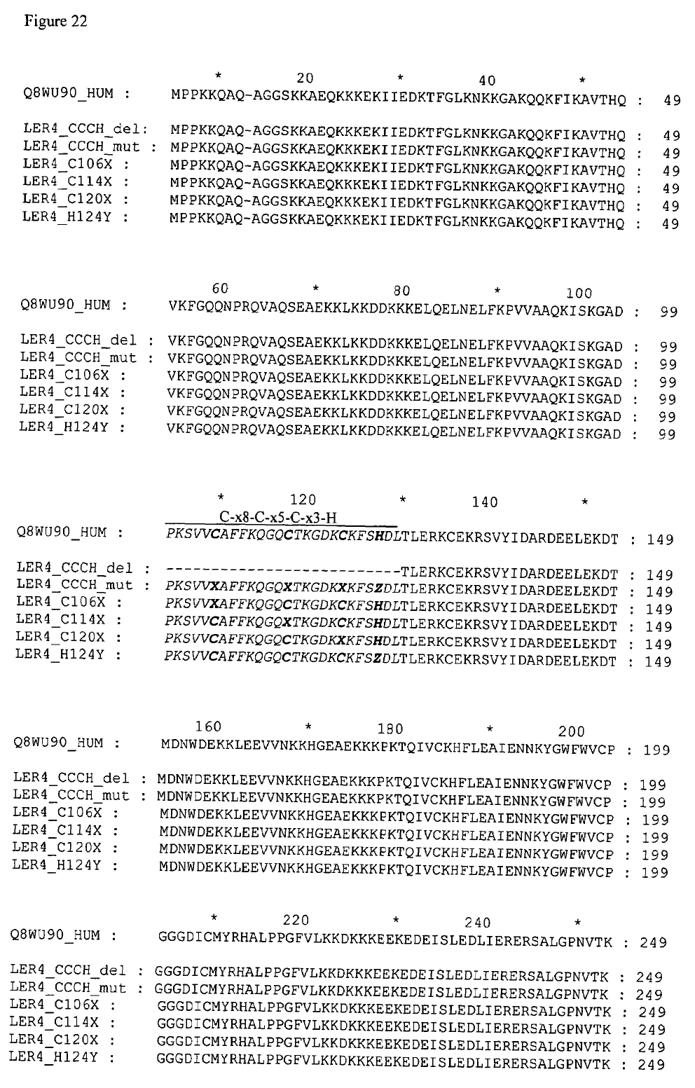

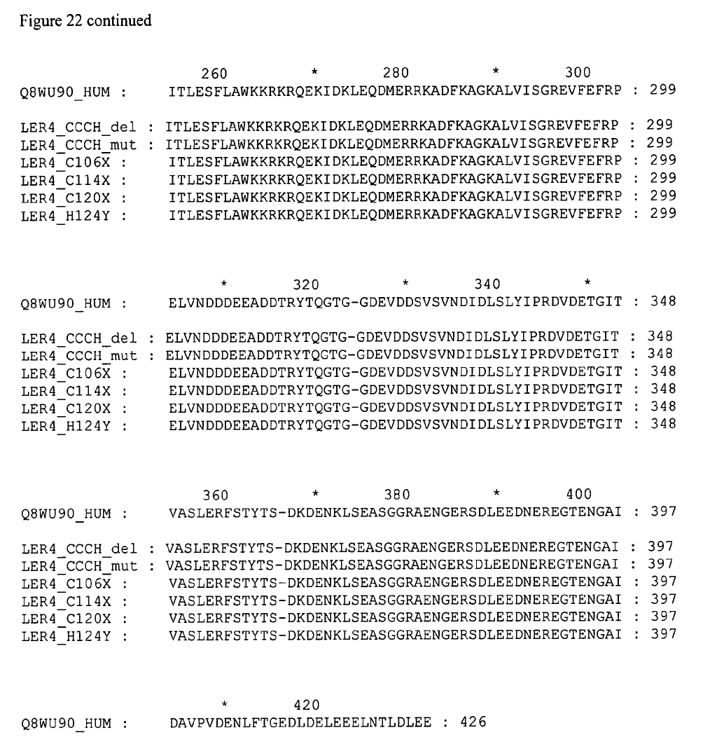

These sequences can be found inter alia in FIG. 22.

The person skilled in the art is well aware how to replace an amino acid in a conserved position in order to obtain a mutant that is likely to result in a loss of function. Such amino acid substitutions may be of conservative or non-conservative nature. Conservative amino acid substitutions relate to the situation where an amino acid is replaced with another amino acid of comparable physico-chemical characteristics. Examples are replacement of glycine for alanin, aspartate for glutamate, Lysine for Asparagine etc.

A non-conserved amino acid substitution refers to the situation where an amino acid is replaced with another amino acid of distinct, if not contrasting physico-chemical properties. Thus, a positively charged amino acid may be replaced by a negatively charged or a hydrophilic amino acid may be replaced by a hydrophobic amino acid. Typical examples constitute the replacement of aspartate by lysine etc.

The person skilled in the art is well aware of how to produce such dominant-negative mutants, in which either parts of the wild-type human LEREPO4 sequence are deleted, or replaced with other amino acid substitutions, since this belongs to standard knowledge in molecular biology.

A protein of SEQ ID No. 7 which differs from wild-type LEREPO4 of SEQ ID No. 4 in amino acids 100 to 125 is thus likely to constitute a dominant-negative proteinaceous mutant of LEREPO4 being capable of interfering with LEREPO4's function in vivo, and thus leading to effects similar to repression of LEREPO4 expression. This applies equally to the functional homologues of LEREPO4.

Further, in case of LEREPO4, it has been shown that TRAF-2 and TRAF-6 interact with the mouse homologue of LEREPO4, namely TCIF (WO02/064786). The binding site of LEREPO4 to these factors can easily be identified using deletion constructs and e.g. surface plasmon resonance analysis. Following the identification of the binding site, non-conservative point mutations within the binding site or LEREPO4 having deletions of this binding site are bona fide dominant negative mutants of LEREPO4.

The above considerations also apply to GliPR and its functional homologues.

Thus, a protein motive search in the above-mentioned databases PROSITE and PFAM revealed that GliPR belongs to the cysteine-rich secretary proteins (CRISP). These proteins comprise the characteristic CRISP domain which is also designated as SCP domain.

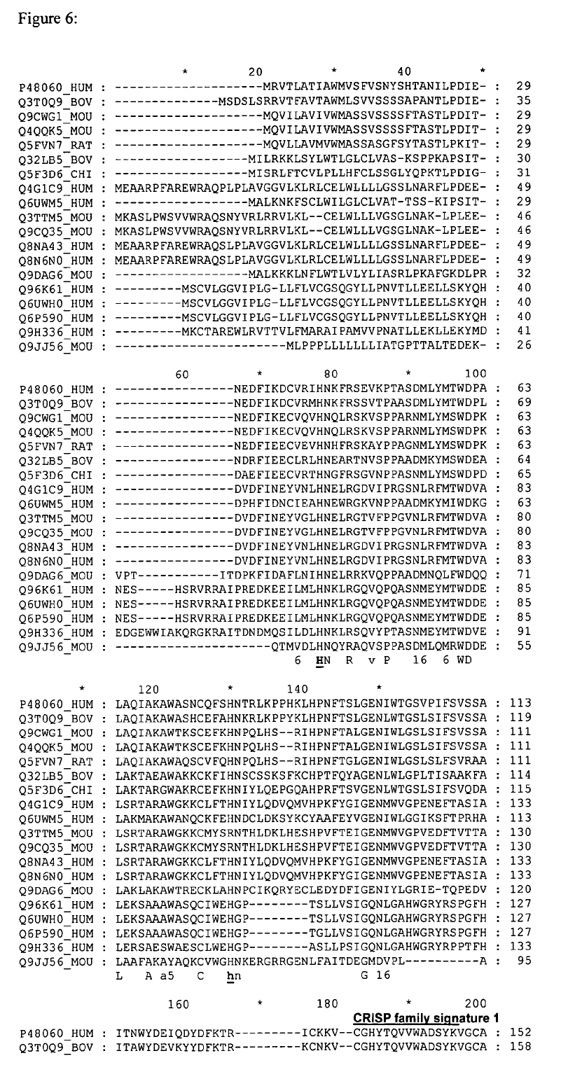

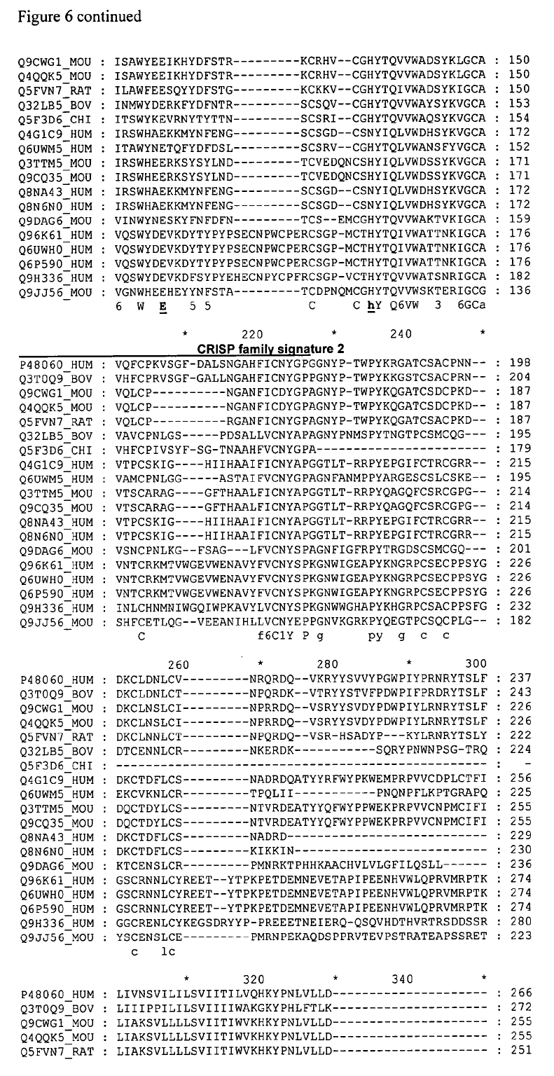

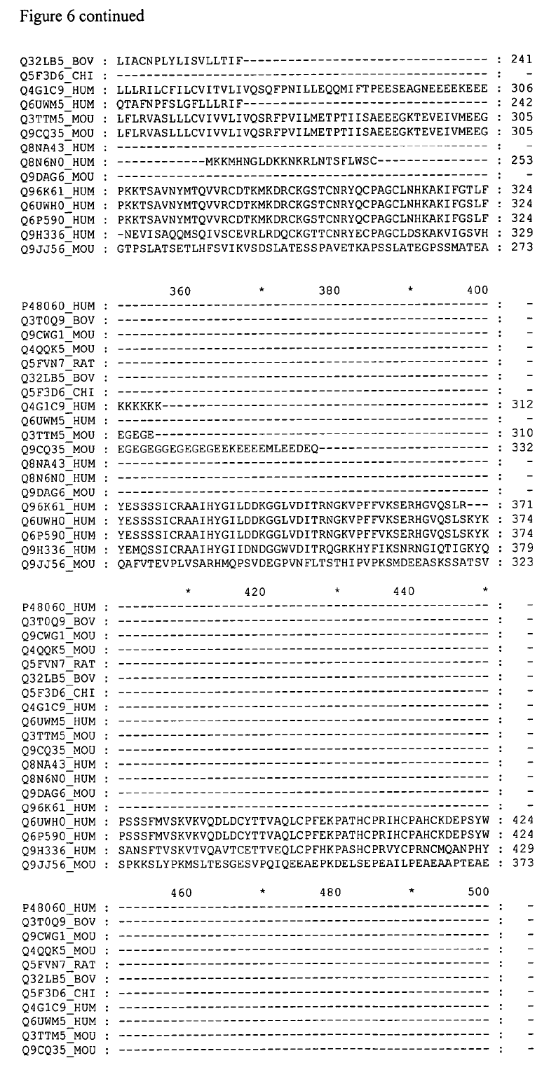

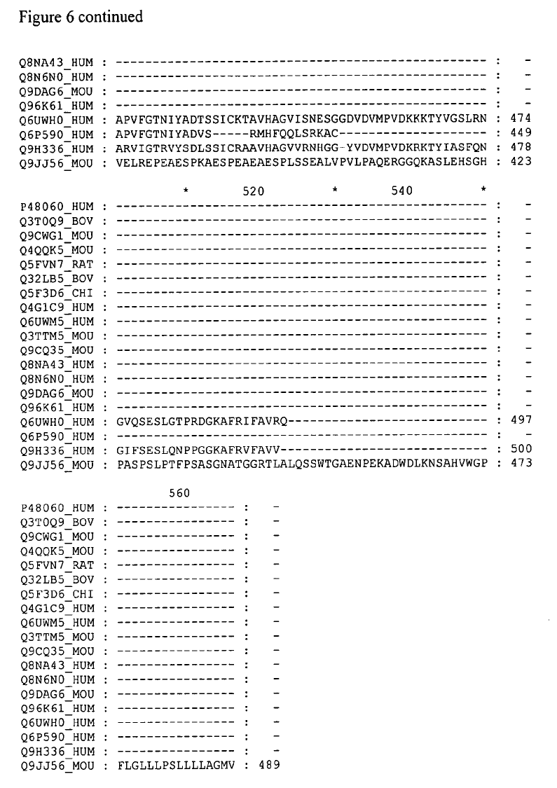

The CRISP family signature 1 in GliPR is given below as SEQ ID No. 8 and the CRISP family signature 2 in GliPR is given as SEQ ID No. 9 Both can be deduced from FIG. 6:

| | |
|---|---|
| SEQ ID No.8: | GHYTQVVWADS |
| SEQ ID No.9: | HFICNYGPGGNY |

Thus, when the amino acid stretch of SEQ ID No. 8 is deleted within SEQ ID No. 6, the resulting sequence may be a putative trans-dominant mutant of GliPR. Similarly, when the amino acid stretch of SEQ ID No. 9 is deleted within SEQ ID No. 6, the resulting sequence is probably a putative trans-dominant mutant of GliPR.

The consensus motif of the CRISP family signature 1 is SEQ ID No. 10:

| | |
|---|---|
| SEQ ID No.10: | [GDER]-[HR]-[FYWH]-[TVS]-[QA]-[LIVM]- |
| | [LIVMA]-W-x(2)-[STN]. |

The consensus motif of the CRISP family signature 2 is SEQ ID No. 11:

| | |
|---|---|
| SEQ ID No.11: | [LIVMFYH]-[LIVMFY]-x-C-[NQRHS]-Y-x- |
| | [PARH]-x-[GL]-N-[LIVMFYWDN]. |

The amino acids in the bracket indicate which amino acids may be present at the respective position. Within GliPR, the CRISP family signature 1 is thus found in amino acids 136-146 and the CRISP family signature 2 is found in amino acids 170-181 of SEQ ID No. 6.

Additionally, using the aforementioned databases, an N-terminal signal sequence (SEQ ID No. 12) as well as a putative transmembrane domain (SEQ ID No. 13) was identified:

| | |
|---|---|
| SEQ ID No.12: | MRVTLATIAWMVSFVSNYSHT |
| SEQ ID No.13: | YTSLFLIVNSVILILSVIITILV |

Thus, when the amino acid stretch of SEQ ID No. 12 is deleted within SEQ ID No. 6, the resulting sequence is a putative trans-dominant mutant of GliPR. Similarly, when the amino acid stretch of SEQ ID No. 13 is deleted within SEQ ID No. 6, the resulting sequence is a putative trans-dominant mutant of GliPR.

In the GliPR protein the signal sequence is located at amino acid positions 1-21 of SEQ ID No. 6, while the transmembrane region is located at amino acid positions 233-255 of SEQ ID No. 6.

Within GliPR, the histidine residue at positions 41, 79 and 137 is conserved. Similarly, the residue glutamate at position 120 is also conserved. The amino acid positions refer to SEQ ID No. 6. If one of these amino acids is substituted in a non-conservative manner, dominant-negative mutants of GliPR may be obtained.

Similarly, point mutants of GliPR which encode for a Lysine or Arginine at position 120 of SEQ ID No. 6 instead of Glutamate are putative dominant negative mutants of GliPR.

Point mutants of GliPR which encode for an Tyrosine, Aspartate or Arginine at position 137 of SEQ ID No. 6 instead of Histidine are putative dominant negative mutants of GliPR. The Histidines at positions 41 and 79 of SEQ ID No. 6 may be mutated the same way.

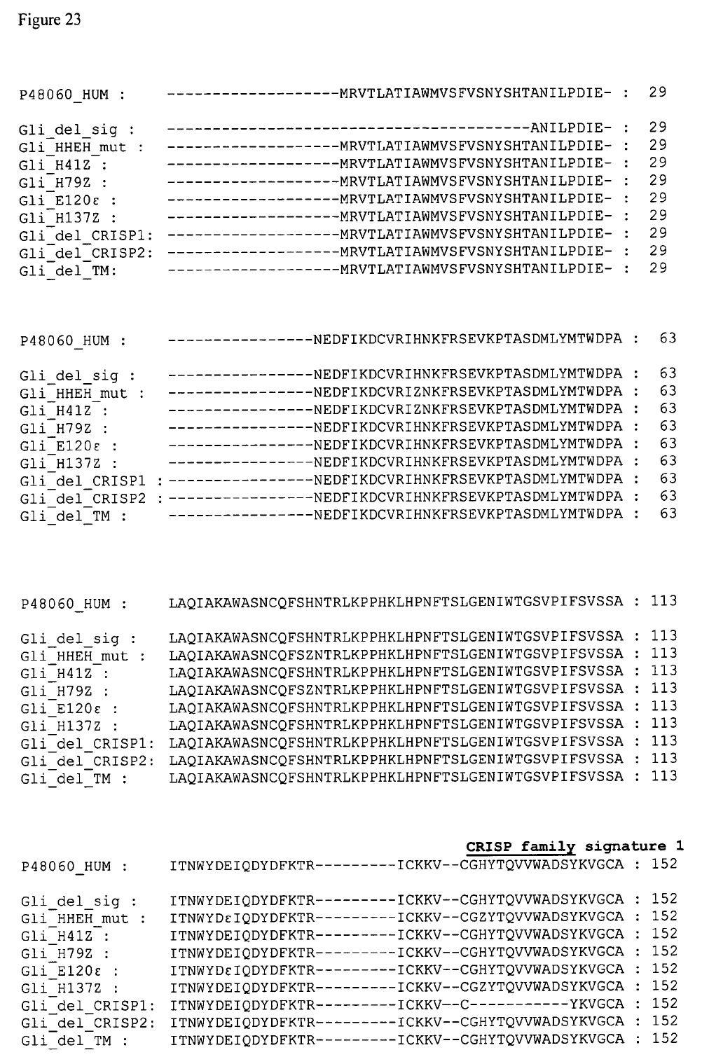

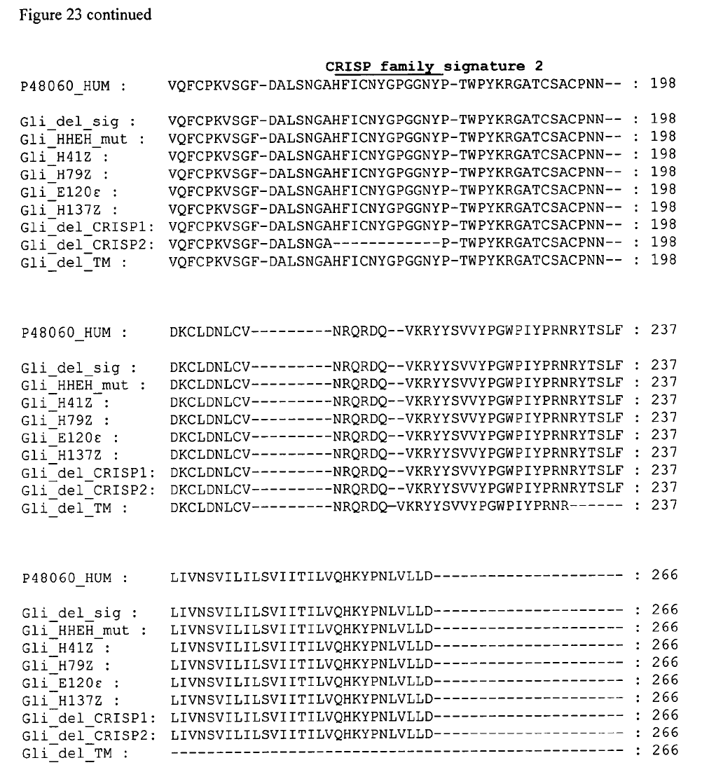

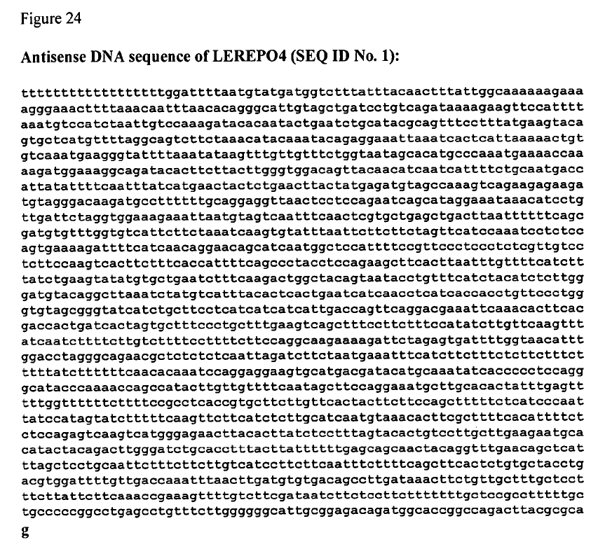

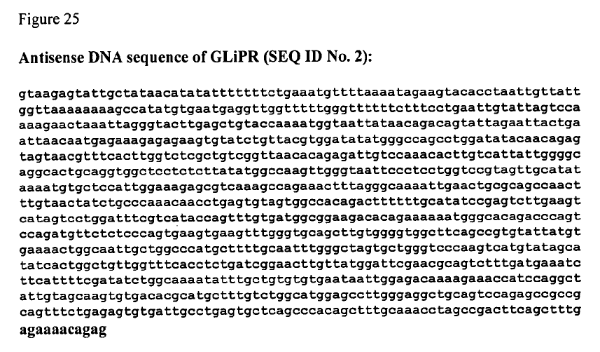

In FIG. 23, various putatively dominant negative mutants are depicted. Thus, Gli_del_sig depicts a sequence in which SEQ ID No. 12 has been deleted in SEQ ID No. 6. Gli_del_CRISP1 depicts a sequence in which SEQ ID No. 10 has been deleted in SEQ ID No. 6. Gli_del_CRISP2 depicts a sequence in which SEQ ID No. 11 has been deleted in SEQ ID No. 6. Gli_del_TM depicts a sequence in which SEQ ID No. 13 has been deleted in SEQ ID No. 6.

Of course, one may also use combinations of the above described sequences to produce dominant negative mutants of GliPR. In FIG. 23, sequences . Gli_HEHH_mut depicts a mutant in which His41, His79, Glu120 and His137 of SEQ ID No. 6 are mutated as described above.

A further embodiment of the present invention relates to recombinant nucleic acid molecules which encode dominant-negative proteinaceous mutants of LEREPO4 or GliPR or their respective functional homologues as described above.

Of course, use of such inhibitory molecules, be it in the form of the dominant-negative proteinaceous mutant or the recombinant nucleic acid molecules encoding therefore for inhibiting LEREPO4 or GliPR function in cellular systems also forms part of the invention.

Yet another object of the present invention relates to pharmaceutical compositions which comprise an inhibitor molecule of LEREPO4 or GliPR function as described above.

The person skilled in the art is well familiar with pharmaceutical formulation technology as far as antisenseRNA, siRNA, vector-based and Protein-based pharmaceutically active compounds are concerned.

Thus, one embodiment of the present invention is directed to pharmaceutical composition comprising at least one inhibitor molecule of LEREPO4's or GliPR's or their respective homologues' function and optionally pharmaceutically acceptable excipients.

Such pharmaceutically acceptable excipients may comprise fillers, anti-stacking agents, lubricants, plasticizers, buffers, stabilizing amino acids, preservatives etc. The precise nature of the excipients will depend on the specific pharmaceutical dosage form comprising the pharmaceutical composition and its way of administration.

The pharmaceutical compositions in accordance with the present invention may be suitable e.g. for rectal, oral, percutaneous, intravenous, intramuscular, inhalative administration or may be administered in the form of an implant. Depending on the dosage forms other embodiments will be considered by the skilled person such as sustained release compositions in the case of oral dosage forms or implantable depot dosage forms. Sustained release dosage forms may be e.g. of the matrix type which may be formed from acrylic polymers, cellulose derivatives etc., of the coating type or the osmotic driven type etc.

In a preferred embodiment the inhibitory molecules of the present invention, particularly in case of antisense RNAs, siRNAs, shRNAs, ribozymes, antisense DNA or vector encoding these RNAs, will be formulated as described in recent reviews (Behlke M A Molecular Therapy (2006), 13: 644-70; Xie F Y et al. Drug Discovery Today. (2006), 11:67-73).

Yet another embodiment of the present invention relates to methods of attenuating, reducing or preventing the transmission or infection of HIV into a cell, wherein the methods comprise the step of applying the aforementioned inhibitory molecules of LEREPO4 or GliPR or their respective functional homologues to a cell or a living individual of human or non-human origin.

Likewise forming part of the invention are methods of attenuating, reducing or preventing the transmission or infection of HIV into a cell comprising the step of providing a pharmaceutical composition as described above to such cells or individuals of human or non-human origin.

The cells may be of human or non-human origin. In one embodiment the cells are isolated and contacted with the inhibitory molecules and pharmaceutical compositions outside the human body.

For the purposes of these methods, the inhibitory molecules or pharmaceutical preparations take a form that allow them to be efficiently delivered into a cell. Once this has been achieved, depending on what type of inhibitory molecule is administered, expression or function of LEREPO4 or GliPR or their respective functional homologues will either be repressed by the applied molecule itself or the applied molecule, e.g. in the case of a virus-based vector will ensure expression of the inhibitor molecule within the cell and thus subsequently lead to repression of LEREPO4 expression or its functional homologues.

Yet another embodiment of the present invention relates to a method of treating or preventing AIDS in an individual of human origin comprising the step of providing to said individual pharmaceutically active amounts of the above-described inhibitory molecules or pharmaceutical preparations, and depending on whether or not this individual has already been infected with HIV or not, prevention or treatment will be achieved.

If the individual has not yet encountered HIV, repression of LEREPO4 or GliPR expression (or of their respective functional homologues) may prevent or at least significantly reduce the likelihood for development of AIDS, as HIV will probably not replicate sufficiently within cells as a consequence of the reduced expression level of LEREPO4 or GliPR or their respective functional homologues.

Where the individual has already been infected with HIV, administration of the inhibitory molecules or the pharmaceutical preparations in accordance with the invention will inhibit, reduce or attenuate further replication of the virus allowing the immune system to gather pace and to actively fight the HIV infection, leading to an improvement of the clinical conditions caused by the HIV infection or by AIDS.

Therefore, one aspect of the present invention relates to the use of the aforementioned inhibitory molecules or for the manufacture of a medicament in the treatment or prevention of HIV infections or the treatment or prevention of AIDS.

Another embodiment of the invention relates to a method of diagnosing an HIV infection and/or AIDS in an individual, comprising the steps of:
obtaining a cellular sample from an individual potentially afflicted with AIDS and/or an HIV infection;
determining the expression level of LEREPO4 and/or GliPR or of their respective functional homologues in said cellular sample, with determination taking place outside the individual's body;
comparing said expression level of LEREPO4 and/or GliPR and/or their respective functional homologues with the expression level of LEREPO4 and/or GliPR and/or their respective functional homologues in a cellular sample obtained from an individual known to be not afflicted with AIDS or a HIV infection;
determining the occurrence of AIDS or HIV infection by observing an increased expression level of LEREPO4 and/or GliPR and/or their respective functional homologues in the individual potentially afflicted with AIDS or an HIV infection compared to the individual known not to have an HIV infection.

The above method of diagnosis may also be used to determine the susceptibility of an individual to the aforementioned methods of treating and preventing HIV infection/AIDS with said inhibitory molecules of pharmaceutical compositions specific to LEREPO4 or GliPR.

The above method of diagnosis may also be used to determine the susceptibility of a specific HIV type, strain or isolate to the aforementioned methods of treating and preventing HIV infection/AIDS with said inhibitory molecules of pharmaceutical compositions specific to LEREPO4 or GliPR.

The above method of diagnosis may also be used to determine the susceptibility of a specific HIV isolate from a specific individual to the aforementioned methods of treating and preventing HIV infection/AIDS with said inhibitory molecules of pharmaceutical compositions specific to LEREPO4 or GliPR.

The above method of diagnosis may also be used to assess the prognosis or prediction of the therapeutic outcome to the aforementioned methods of treating and preventing HIV infection/AIDS with said inhibitory molecules of pharmaceutical compositions specific to LEREPO4 or GliPR by determining LEREPO4 or GliPR expression or by testing HIV isolate, strain, susceptibility to said therapy in vitro or in vivo.

In the context of this method of diagnosis it will be clear to the person skilled in the art that for comparing the different expression levels, normalization between the samples has to be made. Such normalization may, e.g. be made in referencing a standard that is known to have the same expression level in both types of individual, such as actin expression. Furthermore, the person skilled in the art will determine expression of LEREPO4 and/or GliPR and/or their respective functional homologues in the same cell types by the same protocols and methods for the individual which is potentially afflicted with HIV/AIDS and the individual which is known to be not afflicted with HIV/AIDS.

Yet another embodiment of the invention relates to a method of data acquisition comprising the steps of:
determining the expression level of LEREPO4 and/or GliPR and/or their respective functional homologues in the cells of an individual potentially afflicted with AIDS and/or a HIV infection, and optionally
comparing said expression level of LEREPO4 and/or GliPR and/or their respective functional homologues with the expression level of LEREPO4 and/or GliPR and/or their respective functional homologues in a cellular sample obtained from an individual known to be not afflicted with AIDS or HIV infection.

Of course, for the method of data acquisition it will also be crucial to normalize the expression level of LEREPO4 and/or GliPR and/or their respective functional homologues between the different samples investigated by e.g. using such actin expression and use the same cell types, protocols and methods for LEREPO4 and/or GliPR and/or their respective functional homologues for the potentially afflicted vs. the non-afflicted individual.

The person skilled in the art is familiar with the measurement of the expression level of proteins in cellular samples. This may e.g. be done by using antibodies specific for LEREPO4 and/or GliPR and/or their respective functional homologues on the basis of e.g. Western Blotting approaches. Alternatively, real time PCR may be used to measure expression levels as is described in Examples 1, 2 and 3 and the materials and methods section.

Therefore, one aspect of the present invention relates to the use of the expression level of LEREPO4 and/or GliPR and/or their respective functional homologues as an indication of AIDS or an HIV infection in an individual or of the susceptibility to the aforementioned therapies or as a prognostic or predictive biomarker.

A further embodiment of the present invention relates to a method of identifying further inhibitory molecules of LEREPO4's and/or GliPR's and/or their respective functional homologues' function, which are preferably small molecule inhibitors. Such a method may comprise the following steps:
identifying physiological binding partners of LEREPO4 and/or GliPR or at least one of their respective functional homologues;
providing at least one complex between LEREPO4 and/or GliPR or at least one of their respective functional homologues and at least one of its physiological binding partners;
screening said at least one complex against a multitude of compounds and
identifying compounds that disrupt the interactions between LEREPO4 and at least one of its physiological binding partners, GliPR and at least one of its physiological binding partners or between at least one of the functional homologues of LEREPO4 or GliPR and at least one of its physiological binding partners.

Identification of physiological binding partners of LEREPO4 or GliPR and their respective functional homologues may be undertaken using conventional methods. Thus, the person skilled in the art may e.g. obtain an immobilized sample of LEREPO4 or GliPR and their respective functional homologues and incubate this immobilized sample with cellular extracts of mammalian cells such as HeLa cells, NIH3 cells, Jurkat cells etc. Afterwards the immobilized sample may be washed using buffer conditions known to remove non specifically bound factors such as proteins, DNAses lipids, membranes, etc. In a further step, factors that have been specifically interacted with LEREPO4 or GliPR and their respective functional homologues may be eluted from the immobilized sample using e.g. high stringency buffers, i.e. buffers containing high salt concentrations such as 1 M MgCl₂ or chaotropic salts.

In another embodiment, classical two hybrid approaches are undertaken to identify partners of LEREPO4/GliPR or their respective functional homologues.

Once these binding partners have been identified, they are cloned, expressed and a complex between LEREPO4 or GliPR or their respective functional homologues and the newly identified binding partners is formed.

The provision of a complex between LEREPO4 or GliPR and their respective functional homologues and the identified physiological binding partners may again be carried out using conventional methods. Thus, LEREPO4 or GliPR or their respective functional homologues and one of its physiological binding partners may be expressed in e.g. E. coli or eukaryotic cells such as yeast, mammalian cells, etc. and then incubated to obtain a binding complex between the two factors. This complex is subsequently screened against the small molecule library and the ability of the small molecules to interfere with the formation of the complex is measured.

LEREPO4 or GliPR or their respective functional homologues and one of its physiological binding partners may be expressed in e.g. prokaryotic or eukaryotic cells in combination, respectively. Subsequently, LEREPO4 or GliPR or their respective functional homologues may be pulled down in complex with their respective physiological binding partners out of the protein extract by monoclonal antibodies specific to LEREPO4 or GliPR or their functional homologues. Cells with combined expression of these complex forming proteins will be subjected to a screening with small molecules to select such compounds that interfere with the complex formation in cell cultures.

Complexes can then be screened e.g. in a common High Throughput Screen Setup against a multitude of molecules such as small molecules, SELEX libraries, Phage libraries expressing peptides, nucleic acid libraries etc.

A potential inhibitor of LEREPO4 or GliPR or their respective functional homologues will be identified by its ability to disrupt or at least negatively influence the aforementioned complex formation. This may be monitored using conventional methods including inter alia a FRET approach. In this latter approach LEREPO4 or GliPR and their respective functional homologues are labeled with a fluorescent detectable marker. The binding partner is also labeled with a fluorescent detectable marker. Interaction between LEREPO4 or GliPR and their respective functional homologues and their binding partner will bring the markers in close proximity and lead to a specific detectable fluorescent signal. Disruption of the complex by screening in the above described manner will alter this signal. Of course, the skilled person is well familiar with other approaches to detect the effect of a screened compound on the complexed binding partners. Other methods for monitoring binding behaviour of the aforementioned complexes in the presence of potential inhibitors include surface plasmon resonance, HPLC, mass spectroscopy, Seldi-TOF or Maldi-TOF.

A compound that has been identified as being disruptive for the interaction between LEREPO4 or GliPR or their respective functional homologues and one of its physiological binding partners will therefore constitute a good candidate for an inhibitor of LEREPO4 or GliPR or their respective functional homologues function in vivo.

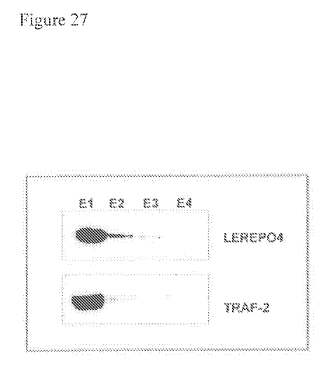

Further, for LEREPO4, a direct interaction with TRAF-2 has been shown (FIG. 27), while it has been reported, that the LEREPO4-homologue in mouse, TCIF, directly interacts with TRAF-2 and TRAF-6 (WO02/064786). Thus, these factors are bona fide binding partners of LEREPO4 and the complex thereof can be used in the above-described High Throughput Screens to identify potential inhibitors of LEREPO4.

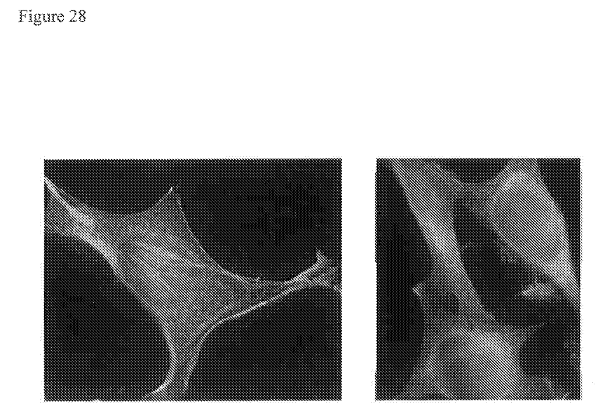

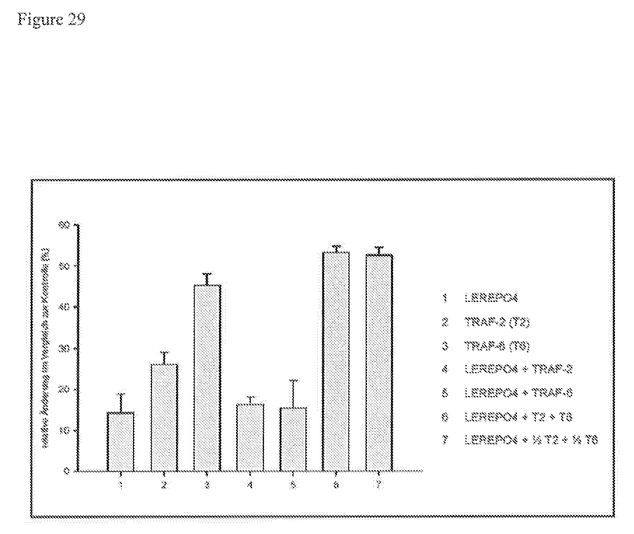

It has also been demonstrated that LEREPO4 is localized to the cytoplasm and functionally interacts with TRAF-2 and -6 to induce the activation of the transcription factor NF-kB (FIGS. 28, 29), which plays a central role in infectious diseases and inflammatory disorders, while it also regulates apoptosis, i.e. activation-induced cell death (AICD) which can lead to ‘immune exhaustion’ during infections.

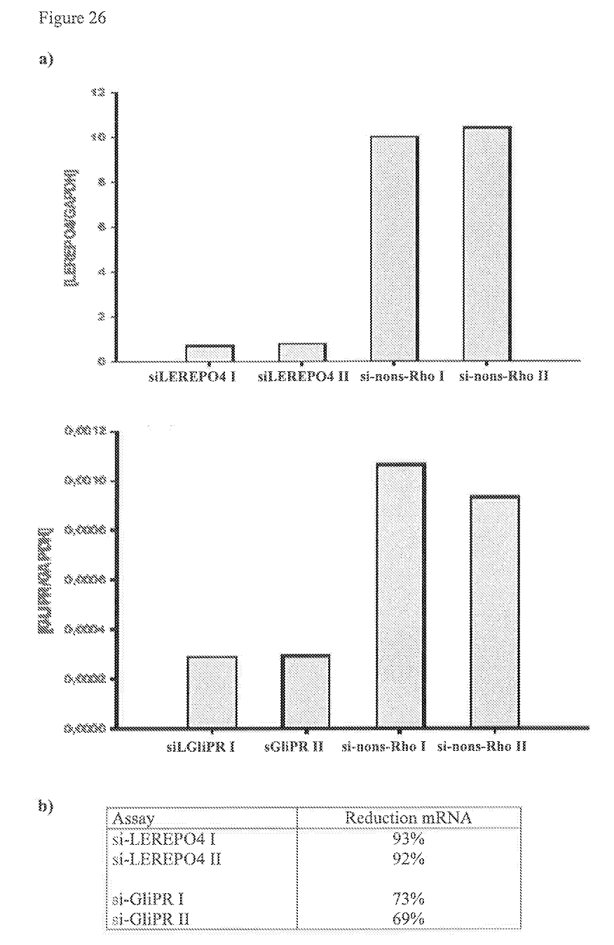

Suppressing the expression of LEREPO4 (FIG. 26) leads to the differential transcriptional regulation of genes as has been demonstrated by a microarray experiment.

TABLE 1
Seleceted genes that are differentially expressed
upon siRNA-mediated knockdown of LEREPO4

| GenBank | | | Gene | fold |
|---|---|---|---|---|
| Acc No. | Probe Set ID | Gene Title | Symbol | induction |
| NM_003246 | 201109_s_at | Thrombospondin 1 | THBS1 | 3.48 |
| NM_000657 | 203685_at | B-cell CLL/ | BCL2 | 3.25 |
| NM_000633 | | lymphoma 2 | | |
| NM_013290 | 213951_s_at | TBP-1 interacting | TBPIP | 3.02 |
| NM_016556 | | protein | | |
| NM_014393 | 204226_at | Staufen homolog 2 | STAU2 | −5.77 |
| NM_000215 | 207187_at | Janus Kinase 3 | JAK3 | −3.19 |
| NM_002675 | 210362_x_at | Promyelocytic | PML | −3.21 |
| NM_033238 | | leukemia | | |
| NM_033239 | | | | |
| NM_033240 | | | | |
| NM_033244 | | | | |
| NM_033246 | | | | |
| NM_033247 | | | | |
| NM_033249 | | | | |
| NM_033250 | | | | |
| NM_002998 | 212157_at | Syndecan 2 | SDC2 | −5.61 |

Following reduction of LEREPO4-expression, the THBS1-gene is transcriptionally upregulated. THBS1 has been shown to play a role during the attachment of HIV-virion, i.e. the binding of viral gp120 to the CD4 surface marker of the target cell, while it has also been shown that THBS1 has anti-angiogenic functions with possible implications for oncogenesis and rheumatoid arthritis.

The BCL2-mRNA is found upregulated following knockdown of LEREPO4. The role of BCL2 has been shown to include the regulation of HIV-replication and the inhibition of apoptosis.

Also the TBPIP-gene appears to become transcriptionally activated upon reduced expression of LEREPO4. It has been demonstrated that TBPIP inhibits the replication of HIV.

The STAU2-specific mRNA is diminished following the reduction of LEREPO4 expression while it has been shown that STAU2 serves as a Cofactor during HIV virion assembly.

The gene encoding JAK3 is transcriptionally repressed upon knockdown of LEREPO4-expression. JAK3 is implicated in a variety of immunity-related phenomena, i.e. retroviral replication, inflammatory disorders (i.e. Psoriasis, rheumatoid arthritis, Spondylarthritis, Lupus erythematodis, Morbus Crohn, Colitis ulcerosa) graft-versus-host-disease, autoimmune diseases (i.e. Diabetis mellitus), rejection of allogenic transplants, allergies, (i.e. Asthma), and also has been shown to play a role in oncogenesis (i.e. acute megakaryoblastic leukemia, acute lymphatic leukemia), and blood coagulation.

The mRNA specific for PML is reduced upon siRNA-mediated knockdown of LEREPO4-expression. It has been shown that PML restricts the replication of HIV.

Also the SDC2-mRNA is found downregulated following knockdown of LEREPO4-expression. It has been shown that SDC2 plays a role in membrane binding and cellular uptake of HIV, but SDC2 is also involved in tumorangiogenesis, i.e. gastrointestinal tumors, colon carcinoma and chronic inflammation, i.e. rheumatoid arthritis, psoriatic arthritis and osteoarthritis.

Yet another embodiment of the present invention relates to methods of attenuating, reducing or preventing infections by viruses, retroviruses, human T cell leukemia virus, Epstein-Barr virus, bacteria, Helicobacter pylori, fungi, parasites, Schistosoma haematobium and/or attenuating, reducing or preventing diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia, wherein the methods comprise the step of applying the aforementioned inhibitory molecules of LEREPO4 or its respective functional homologues to a cell or a living individual of human or non-human origin.

Likewise forming part of the invention are methods of attenuating, reducing or preventing infections by viruses, retroviruses, human T cell leukemia virus, Epstein-Barr virus, bacteria, Helicobacter pylori, fungi, parasites, Schistosoma haematobium and/or attenuating, reducing or preventing diseases, such as, graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia, comprising the step of providing a pharmaceutical composition as described above to such cells or individuals of human or non-human origin.

The cells may be of human or non-human origin. In one embodiment the cells are isolated and contacted with the inhibitory molecules and pharmaceutical compositions outside the human body.

For the purposes of these methods, the inhibitory molecules or pharmaceutical preparations take a form that allow them to be efficiently delivered into a cell. Once this has been achieved, depending on what type of inhibitory molecule is administered, expression or function of LEREPO4 or its respective functional homologues will either be repressed by the applied molecule itself or the applied molecule, e.g. in the case of a virus-based vector will ensure expression of the inhibitor molecule within the cell and thus subsequently lead to repression of LEREPO4 expression or its functional homologues.

Yet another embodiment of the present invention relates to a method of treating or preventing infections by viruses, retroviruses, human T cell leukemia virus, Epstein-Barr virus, bacteria, Helicobacter pylori, fungi, parasites, Schistosoma haematobium, and/or treating or preventing diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia, in an individual of human origin comprising the step of providing to said individual pharmaceutically active amounts of the above-described inhibitory molecules or pharmaceutical preparations, and depending on whether or not this individual has already been symptomatic and/or infected or not, prevention or treatment will be achieved.

If the individual is not symptomatic for graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia, and/or has not yet encountered viruses, retroviruses, human T cell leukemia virus, Epstein-Barr virus, bacteria, Helicobacter pylori, fungi, parasites, Schistosoma haematobium, repression of LEREPO4-expression (or of its respective functional homologues) may prevent or at least significantly reduce the likelihood for development of graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia, and/or may prevent or at least significantly reduce the likelihood for infection by viruses, retroviruses, human T cell leukemia virus, Epstein-Barr virus, bacteria, Helicobacter pylori, fungi, parasites, Schistosoma haematobium and/or may prevent or at least significantly reduce the likelihood for pathologies due to infections by viruses, retroviruses, human T cell leukemia virus, Epstein-Barr virus, bacteria, Helicobacter pylori, fungi, parasites, Schistosoma haematobium, as a consequence of the reduced expression level of LEREPO4 or its respective functional homologues.

Where the individual has already been symptomatic for infections by viruses, retroviruses, human T cell leukemia virus, Epstein-Barr virus, bacteria, Helicobacter pylori, fungi, parasites, Schistosoma haematobium, and/or has been symptomatic for diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia, administration of the inhibitory molecules or the pharmaceutical preparations in accordance with the invention will inhibit, reduce or attenuate further development of symptoms of infections by viruses, retroviruses, human T cell leukemia virus, Epstein-Barr virus, bacteria, Helicobacter pylori, fungi, parasites, Schistosoma haematobium, and/or will inhibit, reduce or attenuate further development of symptoms of diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia, leading to an improvement of the clinical conditions caused by infections by viruses, retroviruses, human T cell leukemia virus, Epstein-Barr virus, bacteria, Helicobacter pylori, fungi, parasites, Schistosoma haematobium, and/or leading to an improvement of the clinical conditions caused by diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia.

Therefore, one aspect of the present invention relates to the use of the aforementioned inhibitory molecules for the manufacture of a medicament in the treatment or prevention of infections by viruses, retroviruses, human T cell leukemia virus, Epstein-Barr virus, bacteria, Helicobacter pylori, fungi, parasites, Schistosoma haematobium, and/or the treatment or prevention of diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia

Another embodiment of the invention relates to a method of diagnosing infections by viruses, retroviruses, human T cell leukemia virus, Epstein-Barr virus, bacteria, Helicobacter pylori, fungi, parasites, Schistosoma haematobium, and/or diagnosing diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia in an individual, comprising the steps of:
obtaining a cellular sample from an individual potentially afflicted with infections by viruses, retroviruses, human T cell leukemia virus, Epstein-Barr virus, bacteria, Helicobacter pylori, fungi, parasites, Schistosoma haematobium, and/or potentially afflicted with diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia;
determining the expression level of LEREPO4 or of its respective functional homologues in said cellular sample, with determination taking place outside the individual's body;
comparing said expression level of LEREPO4 and/or its respective functional homologues with the expression level of LEREPO4 and/or its respective functional homologues in a cellular sample obtained from an individual known to be not afflicted with infections by viruses, retroviruses, human T cell leukemia virus, Epstein-Barr virus, bacteria, Helicobacter pylori, fungi, parasites, Schistosoma haematobium, and/or known to be not afflicted with diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia;
determining the occurrence of infections by viruses, retroviruses, human T cell leukemia virus, Epstein-Barr virus, bacteria, Helicobacter pylori, fungi, parasites, Schistosoma haematobium, and/or the occurrence of diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia by observing an increased expression level of LEREPO4 and/or its respective functional homologues in the individual potentially afflicted with infections by viruses, retroviruses, human T cell leukemia virus, Epstein-Barr virus, bacteria, Helicobacter pylori, fungi, parasites, Schistosoma haematobium, and/or potentially afflicted with diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia compared to the individual known not to have infections by viruses, retroviruses, human T cell leukemia virus, Epstein-Barr virus, bacteria, Helicobacter pylori, fungi, parasites, Schistosoma haematobium, and known not to be symptomatic for diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia.

The above method of diagnosis may also be used to determine the susceptibility of an individual to the aforementioned methods of treating and preventing infections by viruses, retroviruses, human T cell leukemia virus, Epstein-Barr virus, bacteria, Helicobacter pylori, fungi, parasites, Schistosoma haematobium, and/or to determine the susceptibility of an individual to the aforementioned methods of treating and preventing diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia with said inhibitory molecules of pharmaceutical compositions specific to LEREPO4.

The above method of diagnosis may also be used to assess the prognosis or prediction of the therapeutic outcome to the aforementioned methods of treating and preventing infections by viruses, retroviruses, human T cell leukemia virus, Epstein-Barr virus, bacteria, Helicobacter pylori, fungi, parasites, Schistosoma haematobium, and/or to assess the prognosis or prediction of the therapeutic outcome to the aforementioned methods of treating and preventing diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia with said inhibitory molecules of pharmaceutical compositions specific to LEREPO4 by determining LEREPO4 expression in vitro or in vivo.

In the context of this method of diagnosis it will be clear to the person skilled in the art that for comparing the different expression levels, normalization between the samples has to be made. Such normalization may, e.g. be made in referencing a standard that is known to have the same expression level in both types of individual, such as actin expression. Furthermore, the person skilled in the art will determine expression of LEREPO4 and/or its respective functional homologues in the same cell types by the same protocols and methods for the individual which is potentially afflicted with infections by viruses, retroviruses, human T cell leukemia virus, Epstein-Barr virus, bacteria, Helicobacter pylori, fungi, parasites, Schistosoma haematobium, and/or potentially afflicted with diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia and the individual which is known to be not afflicted with viruses, retroviruses, human T cell leukemia virus, Epstein-Barr virus, bacteria, Helicobacter pylori, fungi, parasites, Schistosoma haematobium, and/or known to be not afflicted with diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia.

Yet another embodiment of the invention relates to a method of data acquisition comprising the steps of:
determining the expression level of LEREPO4 and/or its respective functional homologues in the cells of an individual potentially afflicted with infections by viruses, retroviruses, human T cell leukemia virus, Epstein-Barr virus, bacteria, Helicobacter pylori, fungi, parasites, Schistosoma haematobium, diseases and/or potentially afflicted with diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia, and optionally
comparing said expression level of LEREPO4 and/or its respective functional homologues with the expression level of LEREPO4 and/or its respective functional homologues in a cellular sample obtained from an individual known to be not afflicted with infections by viruses, retroviruses, human T cell leukemia virus, Epstein-Barr virus, bacteria, Helicobacter pylori, fungi, parasites, Schistosoma haematobium, and/or known to be not afflicted with diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia.

Of course, for the method of data acquisition it will also be crucial to normalize the expression level of LEREPO4 and/or its respective functional homologues between the different samples investigated by e.g. using such actin expression and use the same cell types, protocols and methods for LEREPO4 and/or its respective functional homologues for the potentially afflicted vs. the non-afflicted individual.

The person skilled in the art is familiar with the measurement of the expression level of proteins in cellular samples. This may e.g. be done by using antibodies specific for LEREPO4 and/or its respective functional homologues on the basis of e.g. Western Blotting approaches. Alternatively, real time PCR may be used to measure expression levels as is described in Examples 1, 2 and 3 and the materials and methods section.

Therefore, one aspect of the present invention relates to the use of the expression level of LEREPO4 and/or its respective functional homologues as an indication of infections by viruses, retroviruses, human T cell leukemia virus, Epstein-Barr virus, bacteria, Helicobacter pylori, fungi, parasites, Schistosoma haematobium, and/or as an indication of diseases, such as graft-vs-host reactions, inflammatory disorders, chronic inflammatory diseases, Psoriasis, rheumatoid arthritis, psoriatic arthritis, osteoarthritis, Spondylarthritis, Lupus erythematodis, Morbus Crohn, Colitis ulcerosa, autoimmune diseases, Diabetis mellitus, rejection of allogenic transplants, blood coagulation disorders, allergies, Asthma, diseases caused by activation induced cell death (AICD), HIV-associated neoplasias, retrovirus-associated neoplasias, virus-associated neoplasias, bacteria-associated neoplasias, parasite-associated neoplasias, neoplasias, glioblastoma, taxan-resistant tumors, gastrointestinal tumors, colon carcinoma, bladder carcinoma, MALT lymphoma, Burkitt lymphoma, adult T cell leukemia, adult T cell lymphoma, acute megakaryoblastic leukemia, acute lymphatic leukemia in an individual or of the susceptibility to the aforementioned therapies or as a prognostic or predictive biomarker.

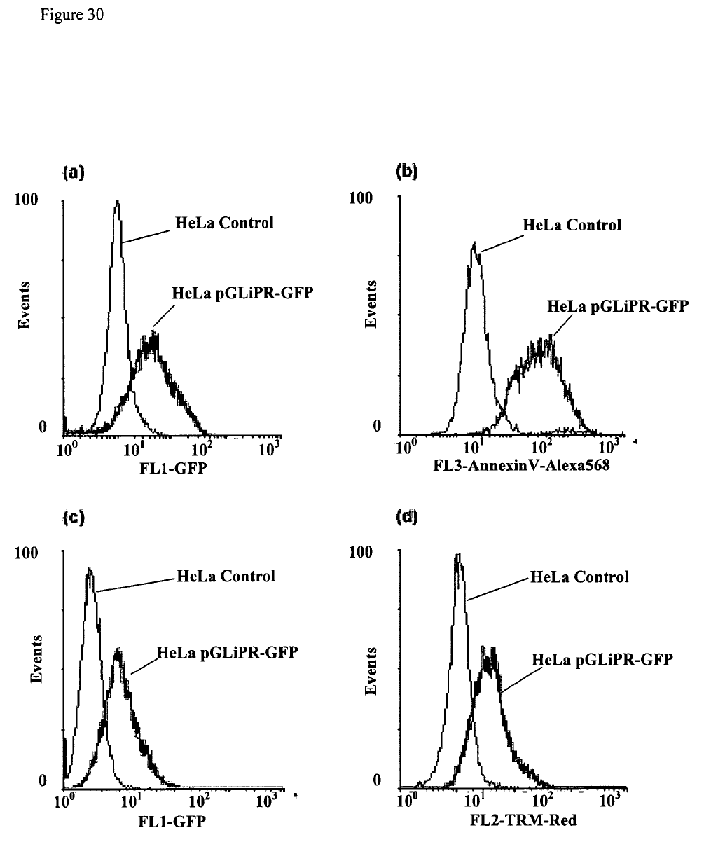

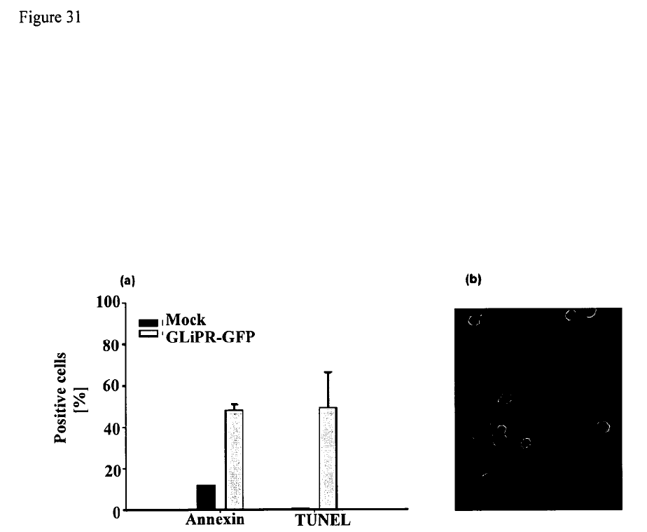

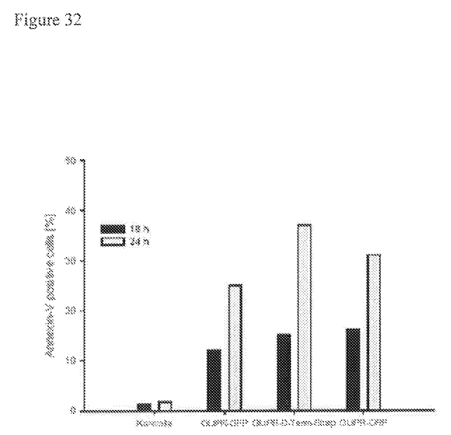

Further, for GliPR, belonging to the superfamily of evolutionary conserved pathogenesis-related (PR−) proteins with a role in viral, bacterial, fungal and parasitical infections, it has been demonstrated that ectopic expression elicits apoptotic cell death (FIG. 30-32), which is a common pathology during infections, i.e. bystander-apoptosis during HIV infections, and can lead to exhaustion of immune cells. An excessive, pathological apoptosis-rate is also associated with acute and chronic neurodegenerative disorders such as trauma of the central nervous system, ischemia of the central nervous system, Morbus Parkinson, Morbus Alzheimer, neuromuscular diseases such as amyotrophic lateral sclerosis, degeneration of the retina, cardiovascular disorders, and autoimmune diseases, i.e. autoimmune-hepatitis.

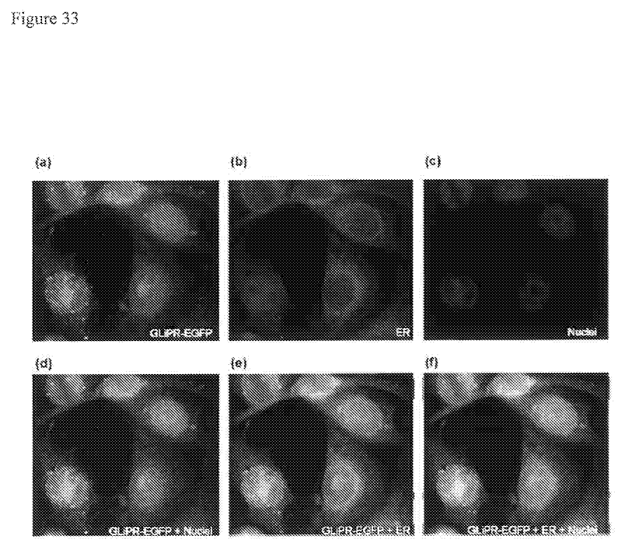

Also, it has been shown that GliPR has a perinuclear localization (FIG. 33) and is a member of the ER/Golgi-resident CRISP-protein family, who play a role in pathologies associated with transmissible spongiform encephalopathies i.e. autophagy-induced neurodegeneration.

Furthermore, it has been demonstrated that GliPR is strongly expressed in monocytes/macrophages and glioma cells, and that inhibition of GliPR-expression leads to inhibition of tumor-growth, -survival and -invasiveness, which points to a central role of GliPR for oncogenesis, i.e. in monoblastic/monocytic acute myelocytic leukemia and glioma.

Suppressing the expression of GliPR leads to the differential transcriptional regulation of genes as has been demonstrated by a microarray experiment.

TABLE 2
Seleceted genes that are differentially expressed
upon siRNA-mediated knockdown of GliPR.

| GenBank | | | Gene | fold |
|---|---|---|---|---|
| Acc No. | Probe Set ID | Gene Title | Symbol | induction |
| NM_002731 | 202741_at | Protein kinase, | PRKACB | −3.52 |
| NM_182948 | | cAMP dependent, | | |
| NM_207578 | | catalytic, β | | |
| NM_006534 | 207700_s_at | Nuclear receptor | NCOA3 | −6.53 |
| NM_181659 | | coactivator 3 | | |
| NM_002998 | 212157_at | Syndecan 2 | SDC2 | −20.23 |
| | 212154_at | | | −3.05 |
| | 212158_at | | | −16.54 |
| NM_000611 | 212463_at | CD59 antigen | CD59 | −3.11 |
| NM_203329 | | | | |
| NM_203330 | | | | |
| NM_203331 | | | | |
| NM_002737 | 215195_at | Protein kinase | PRKCA2 | −4.09 |
| | 213093_at | C, alpha | | −3.55 |

Following reduction of GliPR-expression, the genes encoding for the β-catalytic subunit of the cAMP-dependent proteinkinase (PRKACB) and for proteinkinase C alpha (PRKCA) are transcriptionally downregulated. Both kinases phosphorylate HIV-polypeptides, i.e. pr55, p17, Tat, p24 and play a role during HIV-infection and -pathogenesis.

The expression of SDC2-specific mRNA is reduced upon knockdown of GliPR-expression. The role of SDC2 in membrane binding and cellular uptake of HIV, but also in tumorangiogenesis, i.e. gastrointestinal tumors, colon carcinoma and chronic inflammation, i.e. rheumatoid arthritis, psoriatic arthritis and osteoarthritis has been discussed above.

The NCOA3-gene appears to become transcriptionally silenced following reduction of GliPR-expression and NCOA3 is considered to function as an oncogene in a variety of neoplastic disorders.

Also the mRNA specific for the CD59-antigen is diminished upon knockdown of GliPR-expression. CD59 plays a role in the resistance of neoplasias towards treatment with monoclonal antibodies, and is also involved in the pathological activation of T cells.

Yet another embodiment of the present invention relates to methods of attenuating, reducing or preventing infections by viruses, retroviruses, human T cell leukemia virus, Epstein-Barr virus, bacteria, Helicobacter pylori, fungi, parasites, Schistosoma haematobium, prions and/or attenuating, reducing or preventing diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies, wherein the methods comprise the step of applying the aforementioned inhibitory molecules of GliPR or its respective functional homologues to a cell or a living individual of human or non-human origin.

Likewise forming part of the invention are methods of attenuating, reducing or preventing infections by viruses, retroviruses, human T cell leukemia virus, Epstein-Barr virus, bacteria, Helicobacter pylori, fungi, parasites, Schistosoma haematobium, prions and/or attenuating, reducing or preventing diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies, comprising the step of providing a pharmaceutical composition as described above to such cells or individuals of human or non-human origin.

The cells may be of human or non-human origin. In one embodiment the cells are isolated and contacted with the inhibitory molecules and pharmaceutical compositions outside the human body.

For the purposes of these methods, the inhibitory molecules or pharmaceutical preparations take a form that allow them to be efficiently delivered into a cell. Once this has been achieved, depending on what type of inhibitory molecule is administered, expression or function of GliPR or its respective functional homologues will either be repressed by the applied molecule itself or the applied molecule, e.g. in the case of a virus-based vector will ensure expression of the inhibitor molecule within the cell and thus subsequently lead to repression of GliPR expression or its functional homologues.

Yet another embodiment of the present invention relates to a method of treating or preventing infections by viruses, retroviruses, human T cell leukemia virus, Epstein-Barr virus, bacteria, Helicobacter pylori, fungi, parasites, Schistosoma haematobium, prions and/or treating or preventing diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies, in an individual of human origin comprising the step of providing to said individual pharmaceutically active amounts of the above-described inhibitory molecules or pharmaceutical preparations, and depending on whether or not this individual has already been symptomatic and/or infected or not, prevention or treatment will be achieved.

If the individual is not symptomatic for diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies, and/or has not yet encountered viruses, retroviruses, human T cell leukemia virus, Epstein-Barr virus, bacteria, Helicobacter pylori, fungi, parasites, Schistosoma haematobium, prions, repression of GliPR-expression (or of its respective functional homologues) may prevent or at least significantly reduce the likelihood for development of diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies, and/or may prevent or at least significantly reduce the likelihood for infection by viruses, retroviruses, human T cell leukemia virus, Epstein-Barr virus, bacteria, Helicobacter pylori, fungi, parasites, Schistosoma haematobium, prions and/or may prevent or at least significantly reduce the likelihood for pathologies due to infections by viruses, retroviruses, human T cell leukemia virus, Epstein-Barr virus, bacteria, Helicobacter pylori, fungi, parasites, Schistosoma haematobium, prions as a consequence of the reduced expression level of GliPR or its respective functional homologues.

Where the individual has already been symptomatic for infections by viruses, retroviruses, human T cell leukemia virus, Epstein-Barr virus, bacteria, Helicobacter pylori, fungi, parasites, Schistosoma haematobium, prions, and/or has already been symptomatic for diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies, administration of the inhibitory molecules or the pharmaceutical preparations in accordance with the invention will inhibit, reduce or attenuate further development of symptoms of infections by viruses, retroviruses, human T cell leukemia virus, Epstein-Barr virus, bacteria, Helicobacter pylori, fungi, parasites, Schistosoma haematobium, prions, and/or will inhibit, reduce or attenuate further development of symptoms of diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies, leading to an improvement of the clinical conditions caused by infections by viruses, retroviruses, human T cell leukemia virus, Epstein-Barr virus, bacteria, Helicobacter pylori, fungi, parasites, Schistosoma haematobium, prions, and/or leading to an improvement of the clinical conditions caused by diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies.

Therefore, one aspect of the present invention relates to the use of the aforementioned inhibitory molecules for the manufacture of a medicament in the treatment or prevention of infections by viruses, retroviruses, human T cell leukemia virus, Epstein-Barr virus, bacteria, Helicobacter pylori, fungi, parasites, Schistosoma haematobium, prions, and/or the use of the aforementioned inhibitory molecules for the manufacture of a medicament in the treatment or prevention of diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies.

Another embodiment of the invention relates to a method of diagnosing infections by viruses, retroviruses, human T cell leukemia virus, Epstein-Barr virus, bacteria, Helicobacter pylori, fungi, parasites, Schistosoma haematobium, prions and/or diagnosing diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies in an individual, comprising the steps of:
obtaining a cellular sample from an individual potentially afflicted with infections by viruses, retroviruses, human T cell leukemia virus, Epstein-Barr virus, bacteria, Helicobacter pylori, fungi, parasites, Schistosoma haematobium, prions, and/or potentially afflicted with diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies;
determining the expression level of GliPR or of its respective functional homologues in said cellular sample, with determination taking place outside the individual's body;
comparing said expression level of GliPR and/or its respective functional homologues with the expression level of GliPR and/or its respective functional homologues in a cellular sample obtained from an individual known to be not afflicted with infections by viruses, retroviruses, human T cell leukemia virus, Epstein-Barr virus, bacteria, Helicobacter pylori, fungi, parasites, Schistosoma haematobium, prions and/or known to be not afflicted by diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies;
determining the occurrence of infections by viruses, retroviruses, human T cell leukemia virus, Epstein-Barr virus, bacteria, Helicobacter pylori, fungi, parasites, Schistosoma haematobium, prions and the occurrence of diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies by observing an increased expression level of GliPR and/or its respective functional homologues in the individual potentially afflicted with infections by viruses, retroviruses, human T cell leukemia virus, Epstein-Barr virus, bacteria, Helicobacter pylori, fungi, parasites, Schistosoma haematobium, prions and/or potentially afflicted with, diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies compared to the individual known not to have infections by viruses, retroviruses, human T cell leukemia virus, Epstein-Barr virus, bacteria, Helicobacter pylori, fungi, parasites, Schistosoma haematobium, prions and/or known not to be symptomatic for diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies.

The above method of diagnosis may also be used to determine the susceptibility of an individual to the aforementioned methods of treating and preventing infections by viruses, retroviruses, human T cell leukemia virus, Epstein-Barr virus, bacteria, Helicobacter pylori, fungi, parasites, Schistosoma haematobium, prions and/or to determine the susceptibility of an individual to the aforementioned methods of treating and preventing diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies with said inhibitory molecules of pharmaceutical compositions specific to GliPR.

The above method of diagnosis may also be used to assess the prognosis or prediction of the therapeutic outcome to the aforementioned methods of treating and preventing infections by viruses, retroviruses, human T cell leukemia virus, Epstein-Barr virus, bacteria, Helicobacter pylori, fungi, parasites, Schistosoma haematobium, prions and/or assess the prognosis or prediction of the therapeutic outcome to the aforementioned methods of treating and preventing diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies with said inhibitory molecules of pharmaceutical compositions specific to GliPR by determining GliPR expression in vitro or in vivo.

In the context of this method of diagnosis it will be clear to the person skilled in the art that for comparing the different expression levels, normalization between the samples has to be made. Such normalization may, e.g. be made in referencing a standard that is known to have the same expression level in both types of individual, such as actin expression. Furthermore, the person skilled in the art will determine expression of GliPR and/or its respective functional homologues in the same cell types by the same protocols and methods for the individual which is potentially afflicted with infections by viruses, retroviruses, human T cell leukemia virus, Epstein-Barr virus, bacteria, Helicobacter pylori, fungi, parasites, Schistosoma haematobium, prions and/or potentially afflicted with diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies and the individual which is known to be not afflicted with infections by viruses, retroviruses, human T cell leukemia virus, Epstein-Barr virus, bacteria, Helicobacter pylori, fungi, parasites, Schistosoma haematobium, prions and/or known to be not afflicted with diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies.

Yet another embodiment of the invention relates to a method of data acquisition comprising the steps of:
determining the expression level of GliPR and/or its respective functional homologues in the cells of an individual potentially afflicted with infections by viruses, retroviruses, human T cell leukemia virus, Epstein-Barr virus, bacteria, Helicobacter pylori, fungi, parasites, Schistosoma haematobium, prions and/or potentially afflicted with diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies, and optionally
comparing said expression level of GliPR and/or its respective functional homologues with the expression level of GliPR and/or its respective functional homologues in a cellular sample obtained from an individual known to be not afflicted with infections by viruses, retroviruses, human T cell leukemia virus, Epstein-Barr virus, bacteria, Helicobacter pylori, fungi, parasites, Schistosoma haematobium, prions and/or known to be not afflicted with diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia transmissible spongiform encephalopathies.

Of course, for the method of data acquisition it will also be crucial to normalize the expression level of GliPR and/or its respective functional homologues between the different samples investigated by e.g. using such actin expression and use the same cell types, protocols and methods for GliPR and/or its respective functional homologues for the potentially afflicted vs. the non-afflicted individual.

The person skilled in the art is familiar with the measurement of the expression level of proteins in cellular samples. This may e.g. be done by using antibodies specific for GliPR and/or its respective functional homologues on the basis of e.g. Western Blotting approaches. Alternatively, real time PCR may be used to measure expression levels as is described in Examples 1, 2 and 3 and the materials and methods section.

Therefore, one aspect of the present invention relates to the use of the expression level of GliPR and/or its respective functional homologues as an indication of infections by viruses, retroviruses, human T cell leukemia virus, Epstein-Barr virus, bacteria, Helicobacter pylori, fungi, parasites, Schistosoma haematobium, prions and/or an indication of diseases, such as diseases caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies in an individual or of the susceptibility to the aforementioned therapies or as a prognostic or predictive biomarker.

While the objects of the present invention have been set out in general in the above section, specific examples will be given below how to identify e.g. inhibitory siRNA molecules of LEREPO4 function and GliPR function. Based on the experiments provided, clear guidance is given to the person skilled in the art how corresponding e.g. antisense molecules may be isolated. Therefore, the examples attempt to generally illustrate that inhibition of LEREPO4 or GliPR function is indeed indicative of a reduced infectivity/susceptibility of cells, particularly mammalian or human cells, by HIV.

It will be set out below how LEREPO4 and GliPR were identified, how their involvement in replication of HIV was surprisingly identified and confirmed and how inhibitory molecules were designed that are complementary to the complete coding sequence or part thereof of LEREPO4 or GliPR and how reduced replication of HIV as a consequence of administration of these inhibitory molecules to human cells was measured.

However, these examples are illustrative and should not be construed as limiting the invention to the examples only.

### EXPERIMENTS

### Materials and Methods

### Cell Culture and Preparation of Viral Stocks

HeLa cells and P4-CCR5 [Charneau P. et al., J Virol (1992), 66:2814-2820.] cells (HeLa CD4⁺ CCR5 long terminal repeat-LacZ) were cultured in Dulbecco's modified Eagle's medium (Invitrogen, Karlsruhe, Germany). H9 cells, Jurkat cells and C8166 cells were cultured in RPMI 1640 medium (Invitrogen).

All media were supplemented with 10% fetal calf serum (Gibco-BRL, Karlsruhe, Germany), 1% glutamine (Gibco-BRL) and 1% antibiotic solution (penicillin and streptomycin; Gibco-BRL). P4-CCR5 cells were cultured in the presence of 100 μg/ml G418 (PAA Laboratories, Coelbe, Germany) and 1 μg/ml Puromycin (PAA Laboratories).

Transfection and infection of the cells were carried out in the absence of any antibiotics.

The HIV-1 strain Bru was taken from the supernatant fluid of freshly infected H9 cells. Viral titer (TCID₅₀ units/ml) was determined by titration on C8166 cells as described [Osborne R et al., J Virol Methods (1992), 39:15-26.].

Transfection of Synthetic siRNA Oligonucleotides

In order to inhibit expression of LEREPO4 or GliPR, 3 siRNA oligonucleotides were synthesized, respectively (by Ambion, Austin, Tex., USA) as listed in Table 1 below.

As a negative control regarding specific gene suppression and as a control for transfection efficiency of siRNA's, a non-silencing siRNA with no known homology to mammalian genes, which was 5-prime labeled with rhodamine (si-nons-Rho) was used as listed in Table 1 (Qiagen, Hilden, Germany).

As a positive control for suppression of HIV-1 replication, siRNA oligonucleotides with the following sense and antisense sequences specific to HIV-1 p24 were used [Novina C D et al., Nat Med (2002), 8:681-686.]: 5′-GAUUGUACUGAGAGACAGGdtdt-3′ (sense, SEQ ID No. 22), 5′-CCUGUCUCUCAGUACAAUCdtdt-3′ (antisense, SEQ ID No. 21). All siRNAs were purchased as annealed RNA-duplexes.

TABLE 3

| | | |
|---|---|---|
| siRNA Oligonucleotide | siRNA (sense strand) | siRNA (antisense strand) |

| | | |
|---|---|---|
| si-LEREPO4-1 | 5′-GGAGCAAAGCAACAGAAGUtt-3′ | 5′-ACUUCUGUUGCUUUGCUCCtt-3′ |
| | (SEQ ID No.23 | (SEQ ID No.14) |
| si-LEREPO4-2 | 5′-GGCUGUCACACAUCAAGUUtt-3′ | 5′-AACUUGAUGUGUGACAGCCtt-3′ |
| | (SEQ ID No.24) | (SEQ ID No.15) |
| si-LEREPO4-3 | 5′-GGAUGACAAGAAGAAAGAUtt-3′ | 5′-AUCUUUCUUCUUGUCAUCCtt-3′ |
| | (SEQ ID No.25) | (SEQ ID No.16) |
| si-GliPR-1 | 5′-GGUGAAACCAACAGCCAGUtt-3′ | 5′-ACUGGCUGUUGGUUUCACCtc-3′ |
| | (SEQ ID No.26) | (SEQ ID No.17) |
| si-GliPR-2 | 5′-GGACUAUGACUUCAAGACUtt-3′ | 5′-AGUCUUGAAGUCAUAGUCCtg-3′ |
| | (SEQ ID No.27) | (SEQ ID No.18) |
| si-GliPR-3 | 5′-GGUUUGUUUGGGCAGAUAGUtt-3′ | 5′-ACUAUCUGCCCAAACAACCtg-3′ |
| | SEQ ID No.28) | (SEQ ID No.19) |
| si-nons-Rho | 5′-UUCUUCGAACGUGUCACGUtt-3′ | 5′-ACGUGACACGUUCGAAGAAtt-3′ |
| | (SEQ ID No.29) | (SEQ ID No.20) |

24 h before transfection HeLa cells and P4-CCR5 cells were plated in 24-well plates (Corning, Kaiserslautern, Germany) at 5×10⁴ cell per well in Dulbecco's minimal essential medium containing 10% FBS with no antibiotics. Transfections were performed with Lipofectamine 2000 transfection reagent (Invitrogen) with siRNA at a final concentration of 30 nM according to the manufacturer's recommendations.

After incubating for 6 h, the lipid/siRNA complexes were removed and replaced with fresh medium. For further analyses, cells were removed from the culture dish by trypsinization with 100 μl of 0.25% trypsin/0.02% EDTA in PBS (Cambrex, Verviers, Belgium) for 5 min at 37° C., at different time points after transfection. Transfection efficiency was analysed by flow cytometry 24 h after transfection. Data were acquired and analyzed on FACScalibur with Cell Quest software (Becton Dickinson, Heidelberg, Germany). Effects on cellular viability after siRNA treatment were measured using the cell proliferation reagent WST-1 according to the manufacturer's instructions (Roche, Penzberg, Germany).

### HIV-1 Infection

24 h after siRNA transfection P4-CCR5 cells were infected with HIV-1_{Bru}. Cells were infected in triplicate at a multiplicity of infection (MOI) of 0.01 in the presence of 50 μg/ml DEAE-Dextran (Sigma, Taufkirchen, Germany). After incubation for 4 h the cells were washed with PBS and re-fed with fresh medium. Cells and supernatant samples were collected for quantitative PCR analysis, β-galactosidase enzyme assay and HIV-1 p24 antigen ELISA at indicated time points.

### Real Time PCR Quantitation of Viral and Cellular RNA

RNA was extracted using the RNeasy mini kit including treatment with RNase-free DNase I per supplier's instructions (Qiagen, Hilden, Germany). Synthesis of cDNA was carried out using random hexamer primers and Superscript-II RNaseH-reverse transcriptase according to the manufacturer's specifications (Invitrogen, Karlsruhe, Germany).

Total RNA was reversely transcribed into cDNA using the enzyme SuperScript-II (Invitrogen, Karlsruhe, Germany) in a total volume of 25 μl according to the following conditions:

| | |
|---|---|
| total RNA | 5 μg pDN₆ [25 μM] 0.2 μl (random hexamer Primer |
| | pDN₆; Amersham, FreiburgGermany) |
| H₂O | ad 12.5 μl (Fluka, Buchs, CH) |

reaction mix:

| | |
|---|---|
| 5X RT-buffer | 5 μl (Gibco-BRL, Paisley, UK) |
| DTT [0.1 M] | 2.5 μl (Gibco-BRL, Paisley, UK) |
| dNTP-Mix [10 mM] | 1 μl (Gibco-BRL, Paisley, UK) |
| SuperScript-II | 1 μl (Gibco-BRL, Paisley, UK) |
| H₂O | 3 μl (Fluka, Buchs, CH) |

The RNA sample mixed with the random hexamer primers was denatured for 5 min at 70° C. Subsequently, the reaction mix was added and the reverse transcription was carried out for 1 h at 42° C. with subsequent inactivation of the enzyme for 10 min at 70° C. The reaction was performed by a Primus 96 Thermocycler (MWG-Biotech, Ebersberg, Germany).

Real-time PCR was performed in duplicate reactions employing ABI PRISM 7700 (Applied Biosystems, Darmstadt, Germany) with standard conditions (50° C. for 2 min, 95° C. for 10 min and 40 cycles at 95° C. for 15 s and 60° C. for 1 min). The 25 μl PCR included 2.5 μl cDNA, 1× TaqMan® Universal PCR Master Mix (Applied Biosystems), 0.2 μM TaqMan® probe, 0.2 μM forward primer and 0.2 μM reverse primer. Primers and probes were designed using Primer Express v.1.0 software (Applied Biosystems) and were synthesized by Thermohybaid (Ulm, Germany) In order quantitate LEREPO4, GliPR, HIV-pol, GAPDH in cDNA the following primers and probe were used:

TABLE 4

| | | | |
|---|---|---|---|
| Gene | Forward-Primer | Backward-Primer | TaqMan-Sonde |

| | | | |
|---|---|---|---|
| LEREPO4 | GGTGCCATCTGTCTCCGC | TCTGTTGCTTTGCTCCTTTCTTATT | TGCCCCCCAAGAAACAGGCTCA |
| | (SEQ No.30) | (SEQ No.31) | (SEQ No.32) |
| GliPR | TGCCAGACAAAGCATGCGT | GCTGTGTGTGAATAATTGGAGACAA | TCACACTTGCTACAATAGCCTGGATGGTTTC |
| | (SEQ ID No.33) | (SEQ ID No.34) | (SEQ ID No.35) |
| HIV-pol | AATTTCACCAGTACTACGGTTAAGGC | CTTTAATTCTTTATTCATAGA | TGTTGGTGGGCGGGAATCAAGC |
| | (SEQ ID No.36) | TTCTACTACTCCTTG | (SEQ ID No.38) |
| | | (SEQ ID No.37) | |
| GAPDH | GAAGGTGAAGGTCGGAGTC | GAAGATGGTGATGGGATTTC | CAAGCTTCCCGTTCTCAGCC |
| | (SEQ ID No.39) | (SEQ ID No.40) | (SEQ ID No.41) |

All TaqMan probes are labeled with 6-FAM (6-carboxy fluorescein) at the 5′ end, and with TAMRA (6-carboxy tetramethylrhodamine) at the 3′end.

HIV-1-pol cDNA was amplified to quantitate HIV replication. For normalization of all determinations of gene expressions, GAPDH housekeeping gene expression was analyzed. Copy numbers of the several transcripts were calculated by plasmid standard curves, normalized by GAPDH housekeeping gene transcripts. Standard curves were obtained after amplification of 10 to 106 copies of purified plasmids carrying the amplicons of human LEREPO4, GliPR, HIV-1-pol (modified form of plasmid pLAI.2) or human GAPDH.

The real-time quantitative PCR reaction was conducted with the ABI Prism 7000 Sequence Detection System using the ABI Prism 7000 SDS Software (Applied Biosystems, Darmstadt, Germany) and the following reaction conditions:

| | |
|---|---|
| Universal Master Mix | 12.5 μl |
| Forward-Primer [10 μM] | 0.5 μl |
| Backward-Primer [10 μM] | 0.5 μl |
| TaqMan-Sonde [10 μM] | 0.5 μl |
| cDNA | 2.5 μl |
| H₂O | 8.5 μl |

The following temperature cycle scheme was programmed:

| | | | | |
|---|---|---|---|---|
| initial phase | 50° C. | 2 min | | |
| initial denaturing phase | 95° C. | 10 min | | |
| Cyclic denaturing phase | 95° C. | 15 sec | | |
| | | | {close oversize brace} | 45 cycles |
| Hybridisation/elongation phase | 60° C. | 1 min | | |

### β-Gal Staining of Cells

At indicated time points after HIV-1_{Bru} infection P4-CCR5 cells were washed twice with PBS and fixed for 5 min in fixative (0.25% glutaraldehyde in PBS) at room temperature. After two washes with PBS, cells were covered with staining solution (PBS containing 4 mM potassium ferrocyanide, 4 mM potassium ferricyanide, 2 mM MgCl₂, and 0.4 mg/ml of X-Gal [5-bromo-4-chloro-3-indolyl-β-D-galactopyranoside]) and incubated at 37° C. Subsequently plates were washed twice with PBS and numbers of β-Gal-positive (blue) cells were examined microscopically.

### β-Galactosidase Enzyme Assay

Cell lysates were prepared by using Reporter Lysis Buffer (Promega, Mannheim, Germany). To perform 96-well plate β-galactosidase assays 50 μl of cell lysates and 50 μl of 2×β-galactosidase assay buffer (Promega) were mixed and incubated at 37° C. for 30 min. To stop the reaction, 150 μl of 1 M sodium carbonate was added to the mixture and mixed well by vortexing briefly.

Absorbance of the reaction mixture was read immediately at 420 nm. A standard curve was created, using standards between 0 and 6.0×10⁻³ units of Galactosidase (Promega). Reporter assay results were normalized according to relative cell numbers, which were estimated by using the cell proliferation reagent WST-1 according to the manufacturer's instructions (Roche). WST-1 proliferation assay In order to determine the viability, proliferation, metabolic activity and toxic affliction of cells, the WST-1 assay was applied (Roche, Penzberg, Germany). WST-1 is a tetrazolium salt, which is reduced to a water-soluble stain (formazan) in viable cells.

Formazan is formed by mitochondrial dehydrogenases and can be measured by photonetry. The quantity of dye directly correlates with the number of metabolically active, viable cells. Cytotoxic effects can be detected through decreased cell proliferation using WST-1 reagent. The reagent was used according to manufacterer's instructions.

After incubation with WST-1 for 1 h at 37° C., the medium was sampled and the concentration of the dye was measured in an ELISA reader (Microplate reader 550; BioRad, München).

### HIV-1 p24 Antigen ELISA

HIV-1 p24 ELISA was performed using a commercially available kit (Beckmann Coulter, Krefeld, Germany) according to the manufacturer's instructions. For measuring p24 in the supernatants, 100-fold dilutions of the supernatants were used. All ELISA measurements were done in triplicate.

Cloning of Expression pGliPR-EGFP

In order to reveal the subcellular localisation of GliPR, GliPR was expressed as an EGFP fusion protein. Since GliPR contains a putative signal peptide (for secretion) at its N terminus, the fusion with EGFP was designed at its C terminus.

The plasmid p53-EGFP was employed (Clontech, Heidelberg, Germany), from which the p53 gene was removed by restriction endonuclease cuts by BamHI and SacII. The insert of GliPR was generated by PCR without GliPR's stop codon but additional BamHI- and SacII restriction sites using cDNA from total RNA extracted from HeLa cells.

F-SacII-GliPR-EGFP

(SEQ ID No.42)

5′-CCGCGGATGCGTGTCACACTTGCTACAATAG-3′
B-BamHI-GliPR-EGFP

(SEQ ID No.43)

5′-GGATCCGTCCAAAAGAACTAAATTAGGGTACTTGAG-3′

### Generation of a Polyclonal Antibody Against LEREPO4

A computer based analysis of the secondary structure of the protein LEREPO4 was used to identify a suitable peptide sequence for immunization [Janin 1979; Karplus & Schulz 1985; Parker et al. 1986]. Sequence parts with high probability to be exposed, carrying many charged amino acids, which were unique compared to Swissprot, were considered and the following peptide was selected:

| | |
|---|---|
| LEREPO4-C-Term | |
| NH₂-CDELEEELNTLDLEE-COOH | (SEQ ID No.44) |

This peptide was N-terminally connected via a cystein rich linker with the protein carrier Keyhole Limpet Haemocyanin (KLH). This protein was used to immunize 3 rabbits according to the following protocol:

| | | | | |
|---|---|---|---|---|
| 1. day | Primary | intradermal | Freund's complete adjuvant | |
| | immunization | | | |
| 20. day | 1. boost | subcutanous | Freund's incomplete adjuvant | |
| 30. day | 2. boost | subcutanous | Freund's incomplete adjuvant | |
| 40. day | 3. boost | subcutanous | Freund's incomplete adjuvant | |
| 61. day | 4. boost | subcutanous | Freund's incomplete adjuvant | Blood collection |
| 75. day | 5. boost | subcutanous | Freund's incomplete adjuvant | |
| 90. day | — | — | — | Blood collection |

The generation of the antiserum was performed by Pineda Antikörper-Service (Berlin, Germany). The specificity and titer of the antiserum was determined by antigen capture ELISA using the immunization peptide and by Western blot with a recombinant Strep-Tag-LEREPO4 as a positive control.

### Co-Immunoprecipitation

The identification of putative interaction partners of LEREPO4 was achieved by co-immunoprecipitation assays, using the ProFound Co-Immunoprecipitation System according to the suggestions of the manufacturer (Pierce, Rockford, Ill., USA) Polyclonal antibodies directed against LEREPO4 (see above) were covalently coupled to an aldehyde-activated matrix using Sodiumcyanoborhydride (AminoLink Plus Gel, Pierce) and the matrix was loaded into centrifugable columns (Handee Spin Cup Columns, Pierce). Total protein extracts form Jurkat cells were prepared using the commercially available CelLytic-M-buffer due to the specifications of the manufacturer (Sigma-Aldrich, Taufstein). Then, lysates were loaded onto the columns and incubated on a rotating wheel for 3 h at room temperature. Unspecifically bound proteins were removed by washing the columns four times with Modified Dulbecco's PBS (Pierce). Specifically bound protein complexes were eluted by a shift to pH 3 (ImmunoPure IgG Elution Buffer, Pierce) and following neutralization (1 M Tris, pH 9.5; Ambion) of the obtained fraction. The eluted samples were run on SDS-PAGE gels, and specific detection of LEREPO4 and TRAF2 was achieved by immunoblotting, using the appropriate antibodies (see below).

### SDS-Polyacrylamidegelelectrophoresis (SDS-PAGE)

Protein samples were separated using discontinuing SDS-PAGE. Depending on the size of the desired protein the acrylamide-percentage of the separation gel varied between 10% bis 15%. The samples were mixed with SDS-sample buffer (ImmunoPure; Pierce, Rockford, Ill., USA) and heated at 95° C. for 5 min. Routinely, the See Blue Plus2 Pre-Stained (Invitrogen, Karlsruhe) size standard was employed. For gel-casting and electrophoresis the Mini-Protean 3 Electrophoresis System (Bio-Rad, Munich) was used. Gels were run at 30 mA.

Separation gel

| | |
|---|---|
| 30% Acrylamid/0.8% Bisacrylamid | 10-15% (Roth, Karlsruhe) |
| Tris-HCl, pH 8.8 | 385 mM |
| SDS | 0.1% (Sigma-Aldrich, Taufstein) |
| APS | 0.05% (Roth, Karlsruhe) |
| TEMED | 0.035% (Roth, Karlsruhe) |

Stacking gel

| | |
|---|---|
| 30% Acrylamid/0.8% Bisacrylamid | 4% (Roth, Karlsruhe) |
| Tris-HCl, pH 6.8 | 60 mM |
| SDS | 0.1% (Sigma-Aldrich, Taufstein) |
| APS | 0.05% (Roth, Karlsruhe) |
| TEMED | 0.1% (Roth, Karlsruhe) |

SDS-running buffer, pH 8.9

| | |
|---|---|
| Tris-Base | 3% (Sigma-Aldrich, Taufstein) |
| Glycin | 14.4% (Merck, Darmstadt) |
| SDS | 1% (Sigma-Aldrich, Taufstein) |

### Immuno-Blotting

For immuno-blotting, proteins were transferred onto nitrocellulose membranes (Bio-Rad, Munich) using a semi-dry transfer system (OWL Separation Systems, Portsmouth; NH, USA). The electrotransfer was conducted for 60 min bei 1 mA/cm2 membrane. Membranes were blocked for 30 min with 3% (w/v) BSA in PBST (Sigma-Aldrich, Taufstein). Then, membranes were incubated with primary antibody in 3% (w/v) BSA in PBST and secondary antibody in 3% (w/v) BSA in PBST for 60 and 30 min, respectively. Membranes were washed three times with PBST and once with PBS and ECL detection was monitored by autoradiography using X_ray films (AGFA, Mortsel, Belgium). Removal of bound antibody-complexes for another round of detection was performed using Restore Western Blot Stripping Buffer (Pierce, Rockford, Ill., USA) following the suggestions of the manufacturer.

Transfer buffer

| | |
|---|---|
| TrisHCl, pH 8 | 25 mM |
| Glycin | 150 mM (Merck, Darmstadt) |
| Methanol | 20% (v/v) (Riedel-de Haen, Seelze) |

PBST, pH 7.5

| | |
|---|---|
| Na2HPO4 | 8 mM |
| KH2PO4 | 2 mM |
| NaCl | 150 mM |
| Tween-20 | 0.05% (v/v) (Roth, Karlsruhe) |

ECL-Solutions

| | |
|---|---|
| Solution I | |
| TrisHCl, pH 8.5 | 100 mM |
| Luminol | 2.5 mM (Sigma-Aldrich, Taufstein) |
| p-Coumaric acid | 0.4 mM (Sigma-Aldrich, Taufstein) |
| Solution II | |
| TrisHCl, pH 8.5 | 100 mM |
| H2O2 | 0.02% (v/v) (Sigma-Aldrich, |
| Taufstein) | |

Antibodies

| | Species/ | |
|---|---|---|
| Primary antibodies | Isotype | Reference |
| LEREPO4 (polyc.). | rabbit IgG | see above |
| TRAF-2 (polyc.) | rabbit IgG | Santa Cruz |
| Secondary anitbodies | Species | Reference |
| anti-rabbit-IgG-HRP | mouse | Sigma-Aldrich |
| anti-mouse-IgG-HRP | rabbit | Sigma-Aldrich |

Gene-Expression Analysis with Microarrays

DNA-Microarrays were used to determine the effect of siRNA-mediated knockdown of LEREPO4 and GliPR on the gene expression profile of HeLa cells. To this end, total RNA was extracted using the RNeasy mini kit including treatment with RNase-free DNase I per supplier's instructions (Qiagen, Hilden, Germany).

Microarray analyses were a conducted at the University Hospital Frankfurt University in form of a payable service order. Total RNA was reverse transcribed into double stranded cDNA by Oligo dT-priming followed by an in vitro-transcription assay to produce biotinylated cRNA, which was then fragmented by size. The biotinylated cRNAs were hybridized to HG-U133 Plus 2.0 Arrays (Affymetrix, Santa Clara, USA) and signals were visualized by staining with a Streptavidin-Phycoerythrin-conjugate and subsequent fluorescence-detection by laser-scanning.

### Data Analysis of Microarrays

The arrays used, feature 47,000 transcripts, and for each feature 11 probeset-pairs are spotted onto the array. Each pair consists of a perfect match (PM) and a mismatch (MM) Oligonucleotide, that harbors one missense-mutation. A probe set is considered to be ‘positive’, in the sense of specific hybridization, if the PM-fluorescence is higher than the MM-fluorescence, and is considered ‘negative’ if the MM-fluorescence is higher than the PM-fluorescencence. The total number of positive and negative probesets was employed to determine the ‘absolute call’ for a certain transcript. A transcript can therefore be, ‘present’, ‘absent’ or ‘marginal’ in the sense of detection. The average fluorescence signal of a probeset is used also as a measure for the relative expression level, or the ‘average difference’ of a transcript. In general, values for two different experimental conditions (+/−siRNA knockdown of LEREPO4/GliPR) were determined in the microarray experiments. Untreated cells were used as a ‘baseline’ while siRNA treated samples yielded the ‘experimental array’. To use the ‘baseline’ as a reference, normalization procedures had to be employed, including the calculation of overall differences of fluorescence intensities of the two array-sets. Comparison-algorithms were than used to determine, if, after normalization, a given probeset had a stronger or weaker fluorescence-intensity when comparing ‘experiment’- with ‘baseline’-datasets to obtain ‘difference calls’.

Only candidate genes were further analyzed, that had a ‘present’ absolute call and that had a ‘difference call’ representing a fold change of at least ±3.0.

### NF-kB Transcription Factor Assay

An ELISA-based method was used to determine the activity of the transcription factor NF-κB. To this end, 96 well plates containing immobilised DNA-oligonucleotides, harboring the NF-κB-consensus site were used. Nuclear protein extracts were then transferred into the wells. The DNA-bound form of NF-κB can then be detected using a p65-specific antibody and a HRP-conjugated secondary antibody. HeLa cells were transfected with the according expression plasmids (see below) and nuclear protein extracts were prepared using the Nuclear Extract Kit (Active Motif, Belgien) according to the suggestions of the manufacturer. 2.5 μg of extract was used per well. The quantification of NF-κB-activity was performed with the TransAM NF-κB-Transcription Factor Assay (Active Motif, Belgien). As a positive control, nuclear extract from Jurkat cells who had been treated with the phorbolester TPA (50 ng/ml) and the calciumionophor A23187 (0.5 μM) were used. All assays were prepared in duplicate. The layout of the experiment, whose data are depicted in FIG. 29, is given below

| Assay | LEREPO4 | TRAF-2 | TRAF-6 | Vector |
|---|---|---|---|---|
| 1 | 0.8 μg | | | 0.8 μg |
| 2 | | 0.4 μg | | 1.2 μg |
| 3 | | | 0.4 μg | 1.2 μg |
| 4 | 0.8 μg | 0.4 μg | | 0.4 μg |
| 5 | 0.8 μg | | 0.4 μg | 0.4 μg |
| 6 | 0.8 μg | 0.4 μg | 0.4 μg | |
| 7 | 0.8 μg | 0.2 μg | 0.2 μg | 0.4 μg |
| K | | | | 1.6 μg |

The efficiency of transfection was measured after 24 h and 48 h by flow cytometry.

### Flow Cytometry (FACS)

FACS was used to detect the expression of surface markers, i.e., of the transfection marker gene ΔLNGF. Also FACS was used to assay the expression of EGFP-fusion proteins, and to determine the efficiency of siRNA-transfection experiments. As described below, also Annexin V-staining and TUNEL-assays for the detection of apoptosis were analyzed by FACS.

For antibody staining, cells were resuspended in FACS-buffer (1% FCS in PBS). A fluorescein-labelled anti-NGFR-antibody (B D, Heidelberg) in PBS was added to the resuspended cells for 15-30 min at 4° C. Cells were then washed twice with FACS-buffer and fixated using 3% (v/v) Formaldehyde. Fixed cells were kept at 4° C. until the analysis. For detection of EGFP-fusion proteins and fluorescent siRNA oligonucleotides cells were directly resuspended in cold PBS and measured by FACS. A FACScan instrument (BD) equipped with the CellQuest-Software was employed for all analyses.

### Annexin V Staining

One of the earliest detectable alterations of apoptotic cells is the presence of phosphatidylserine (PS) on the cell surface. PS can be stained with the protein Annexin V. Here the Annexin-V-Staining Kit (Roche, Penzberg) was used as suggested by the manufacturer. The kit contains Annexin V coupled to the fluorescent dye Alexa 568. Also necrotic cells take up the Annexin V-conjugate, due to membrane disintegration. To control for the presence of necrotic cells, a staining with propidiumiodide was employed, a dye that is only retained in necrotic cells. To this end, cells were incubated 5 min at room temperature with 1 μg/ml propidiumiodide in PBS. Samples were analyzed by flow cytometry and gating strategies were employed to exclude necrotic cells from the analyses.

### TUNEL-Assay

Another method for cell death detection is the TUNEL-Assay (TdT-mediated x-dUTP nick end labeling), which detects DNA-degradation, a hallmark of early apoptosis. Because of nicking of DNA free 3′-OH-ends are generated which can be labelled with fluorescent nucleotides using the terminale deoxynukleotidyl Transferase (TdT) enzyme. For TUNEL-assays the In Situ Cell Death Detection Kit TMR red (Roche, Penzberg) was employed in accordance with the suggestions of the manufacturer. Cells were then analyzed by flow cytometry.

### Fluorescence Microscopy

Fluorescence microscopy was used to determine the intracellular localization of proteins, and to determine the efficiency of siRNA-transfections. To this end 1×10⁴ to 5×10⁴ adherent growing cells were seeded onto epoxy-coated coverslips (Roth, Karlsruhe) und cultured overnight. Prior to staining, cells were washed with warm PBS and then fixed with for 10 min at room temperature with 3% (v/v) Formaldehyde in PBS, followed by a 10 min incubation with 0.1% (v/v) Triton X-100 in PBS to obtain permeabilization. When necessary, the blocking reagent Image-iT FX Signal Enhancer (Molecular Probes, Eugene, Oreg., USA) was added for 30 min and then cells were incubated at room temperature for 90 min with primary antibody in 3% (w/v) BSA in PBS). Following a washing step with PBS, fluorophore-conjugated secondary antibody was added for 90 min at room temperature. Nuclei were stained with DAPI (4′,6-Diamidino-2-Phenylindol; Molecular Probes) for 5 min at room temperature and the cells were then overlayed with ProLong mounting medium (Molecular Probes). Images were obtained using an Axioplan II fluorescence microscope (Zeiss, Gottingen) equipped with the Axiovision Software (Zeiss).

### Results

### 1. Transcriptional Regulation of Cellular Gene Expression by HIV-1 Infection.

As already set out above, interactions between viral and cellular factors are necessary in order to ensure productive HIV replication within cells. In the context of the present invention, quantitative RT-PCR was used to determine whether infection of P4-CCR5 cells or Jurkat cells with HIV-1_{Bru} (Moi 0.1) leads to a modification of expression of cellular genes such as LEREPO4 and GliPR.

### Quantitative Real Time PCR System

For quantification of LEREPO4 and GliPR specific gene primers and TaqMan probes were designed and applied. The resulting amplified construct was used in each case to carry out a homology comparison by the BLAST program package of the NCBI in order to ensure that the chosen primers and probes would amplify the desired genes only.

Plasmids in which DNA fragments of the corresponding genes had been cloned before were used as amplification standard. For that purpose defined amounts of molecules in dilution steps of 1×10¹ to 1×10⁶ were used for a TaqMan PCR. A standard curve was calculated from these values and used for the subsequent determination of the transcript amount of the gene to be analysed.

For determination of the cellular HIV-RNA-copy number after infection, the TaqMan system was used with a primer pair being specific for pol-region of HIV-1. The transcript of a housekeeping gene (glycerine aldehyde-3-phosphate-dehydrogenase, GAPDH) was used for normalization of the obtained values in each sample. By referencing the copy number of the gene to be analysed to the copy number of GAPDH, the relative transcript amount was determined.

### Modification of Cellular Gene Expression by HIV Infection

Using this TaqMan system it was possible to determine the differential transcriptional regulation of LEREPO4 and GliPR as induced by HIV infection.

P4-CCR5 cells were used for that purpose. These cells as a matter of exogenous expression of the CD4-receptors and the co-receptor CCR5 are injectable with HIV-1. The cells were infected at an MOI of 0.01 with the virus strain HIV-1_{Bru}. Four days after infection, cellular total RNA was isolated and after cDNA synthesis used for TaqMan PCR.

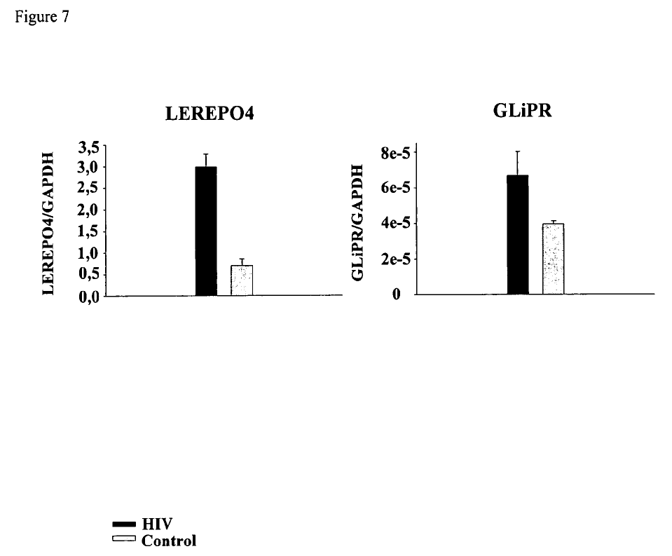

The results are shown in FIG. 7.

Determination of relative transcription of the two genes revealed that as a matter of HIV infection, transcription of LEREPO4 was increased by a factor of 4. In the case of GliPR HIV infection resulted in an induction of transcription by approximately 70%.

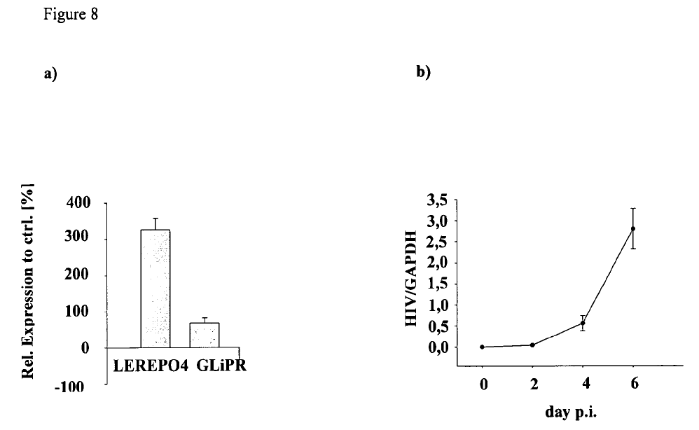

The copy number of HIV RNA during the course of infection was also determined using TaqMan PCR in order to ensure that productive HIV replication was ensured under the experimental conditions. The results are shown in FIG. 8.

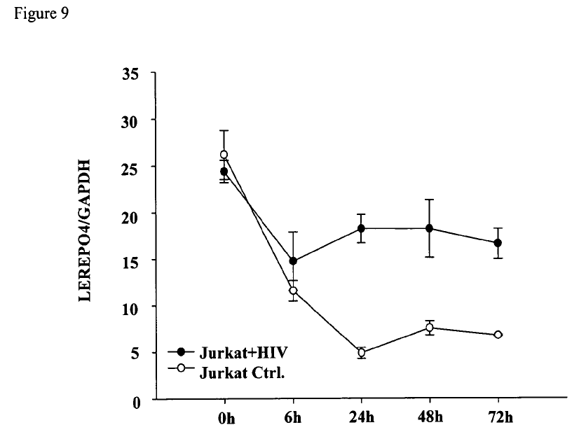

In order to exclude the possibility that induction of LEREPO4 (or GliPR) transcription is a cell-type specific artefact, transcriptional regulation was also determined for the Jurkat CD4⁺-T-cell line. For that purpose, Jurkat cells were infected at an MOI of 0.2 with HIV-1_{Bru}. Total cellular RNA was isolated after 6, 24, 48 and 72 hours and used for TaqMan PCR. The results are shown in FIG. 9.

The results confirmed that an induction of LEREPO4 transcription is also observed if different cell types are used.

In summary, transcription of LEREPO4 and GliPR as determined by real time PCR is increased as a consequence of HIV infection.

### 2. Post-Transcriptional Gene Suppression Using RNA Interference

In order to down regulate LEREPO4 and GliPR function in vivo, an RNA interference (RNAi) approach was taken.

Short hairpin RNA (shRNA) and synthetic double-stranded RNA oligonucleotides (synthetic siRNA) were used for that purpose. The effect of this gene repression by using these RNAs on HIV replication was determined using known HIV infection tests.

In the present case, synthetic siRNA oligonucleotides were used to silence, or at least reduce expression of LEREPO4 and GliPR.

For this purpose, siRNA sequences were identified, as set out in the specification. In detail, the following sequences were selected for LEREPO4:

| | |
|---|---|
| si-LEREPO4-1: | |
| antisense | |
| 5′-ACUUCUGUUGCUUUGCUCCtt-3′: | (SEQ ID No.14) |
| sense | |
| 5′-GGAGCAAAGCAACAGAAGUtt-3′ | (SEQ ID No.23) |
| si-LEREPO4-2: | |
| antisense | |
| 5′-AACUUGAUGUGUGACAGCCtt-3′: | (SEQ ID No.15) |
| sense | |
| 5′-GGCUGUCACACAUCAAGUUtt-3′: | (SEQ ID No.24) |
| si-LEREPO4-3: | |
| antisense | |
| 5′-AUCUUUCUUCUUGUCAUCCtt-3′ | (SEQ ID No.16) |
| sense | |
| 5′-GGAUGACAAGAAGAAAGAUtt-3′ | (SEQ ID No.25) |
| si-GliPR-1: | |
| antisense | |
| 5′-ACUGGCUGUUGGUUUCACCtc-3′ | (SEQ ID No.17) |
| sense | |
| 5′-GGUGAAACCAACAGCCAGUtt-3′ | (SEQ ID No.26) |
| si-GliPR-2: | |
| antisense | |
| 5′-AGUCUUGAAGUCAUAGUCCtg-3′ | (SEQ ID No.18) |
| sense | |
| 5′-GGACUAUGACUUCAAGACUtt-3′ | (SEQ ID No.27) |
| si-GliPR-3: | |
| antisense | |
| 5′-ACUAUCUGCCCAAACAACCtg-3′ | (SEQ ID No.19) |
| sense | |
| 5′-GGUUGUUUGGGCAGAUAGUtt-3′ | (SEQ ID No.28) |
| si-nons-Rho:: | |
| antisense | |
| 5′-ACGUGACACGUUCGAAGAAtt-3′ | (SEQ ID No.20) |
| sense | |
| 5′-UUCUUCGAACGUGUCACGUtt-3′ | (SEQ ID No.29) |

Sequences of SEQ ID No. 14 to 16 and 23 to 25 were specific for LEREPO4, sequences of SEQ ID No. 17 to 19 and 26 to 28 were specific for GliPR. SEQ ID Nos 20 and 29 were used as the negative control. In each case the 100% complementary sequences which were used as antisense sequences hybridized to the sense strand as siRNA (see also Table 3). Each antisense sequence had also a 3′overhang with tt or tc or tg (see also Table 3).

In order to determine the effect of repression of LEREPO4 and GliPR on HIV infectivity, the above-described double stranded siRNAs were transfected into cells.

### Transfection Conditions

First, optimised transfection conditions were established. Furthermore, an si concentration was chosen, for which off-target effects were reduced as far as possible. An off-target effect is an unspecific gene suppression as a consequence of siRNA transfection.

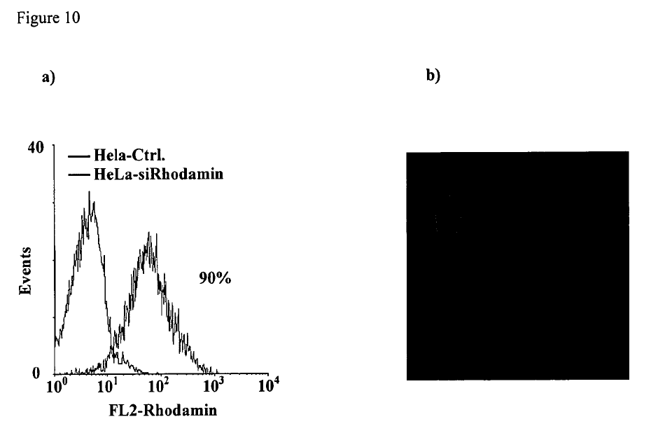

The optimal transfection conditions were determined using Rhodamin-labelled non-silencing siRNA (si-nons-Rho). Different transfection reagents and different transfection conditions were tested. Analysis of the achieved transfection efficiency was determined using FACS analysis or fluorescence microscopy. The results are shown in FIG. 10.

Optimal transfection was achieved using Lipofectamine 2000 (Invitrogen) in the presence of OptiMem medium (Invitrogen). The concentration of siRNAs during transfection was generally approximately 30 nM. Using these conditions transfection efficiencies of up to 90% were achieved (see FIG. 10).

### Proliferation Rate

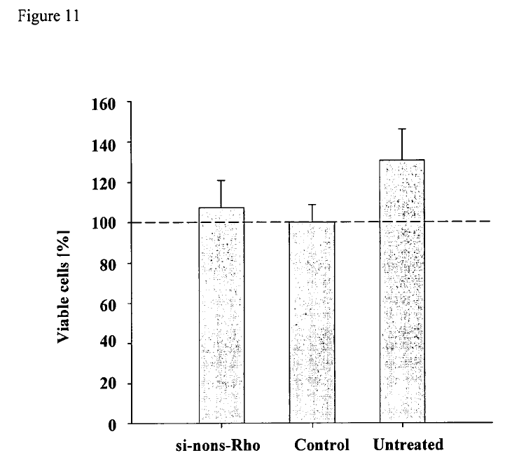

It was also investigated whether such transfection of siRNA molecules would be cytotoxic for the target cells. For that purpose, the proliferation rate of the cells was determined after transfection. In order to determine the proliferation rate, the WST-1 cell proliferation reagent (Roche, Penzberg) was used. Cell proliferation of HeLa cells was determined immediately before an siRNA transfection. This value was used for normalization of the cell viability (which was) calculated later. In order to determine cell viability, proliferation was determined 24 hours after transfection. Cells which had been transfected with transfection reagent, but without siRNA molecules, were used as control. The resulting value obtained was taken as 100% viable cells. In comparison, the WST-1 turnover was determined after transfection with si-nons-Rho molecules. Furthermore, non-treated cells were used as a control. The results are shown in FIG. 11.

No significant difference was determined for the cell proliferation between transfected and control cells.

### Quantitative Real Time PCR

After optimisation of siRNA transfection, the inhibitory effect of the specific siRNA oligonucleotides directed against LEREPO4 and GliPR was determined.

For that purpose HeLa cells were transfected with the respective siRNA oligonucleotides. Rhodamin-labelled non-silencing siRNA molecules were used as a control. Furthermore, this latter siRNA was used as a control of transfection efficiency, as it was reasonable to assume that comparable transfection efficiency was achieved with the LEREPO4 or GliPR siRNA oligonucleotides.

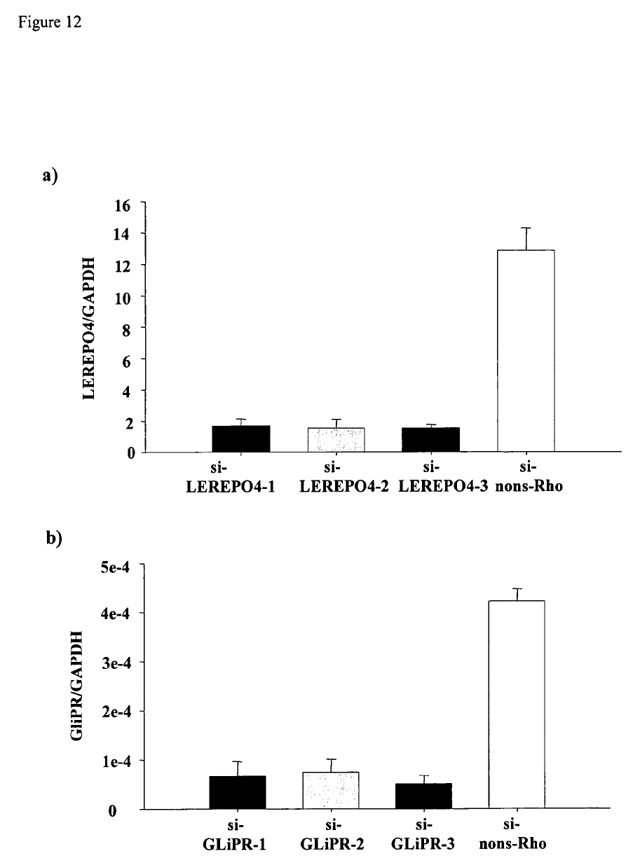

As described in the above, cellular RNA was isolated 24 hours after transfection and used for cDNA synthesis. Then real time TaqMan PCR was performed which allowed determination of the extent of repression of LEREPO4 and GliPR. The results are shown in FIG. 12.

and are further summarized in Table 5 below.

| SiRNA | Reduction mRNA (%) |
|---|---|
| si-LEREPO4-1 | 87 |
| si-LEREPO4-2 | 88 |
| si-LEREPO4-3 | 88 |
| si-GliPR-1 | 84 |
| si-GliPR-2 | 82 |
| si-GliPR-3 | 88 |

It becomes clear from FIG. 12 that the above-described synthetic siRNAs led to a significant reduction of the respective target mRNAs. All three siRNA molecules directed against LEREPO4 led to a reduction of the target RNA of about 88% in comparison to the control. Furthermore, the siRNAs directed against GliPR led to a suppression of the target mRNA of about 85%.

Effect of siRNA-Mediated Gene Suppression on Protein Level

To further confirm that transfection of LEREPO4- and GliPR-specific siRNAs not only led to suppression of the respective target mRNAs, but also to reduced expression of the protein, FACS analysis and Western Blot were performed.

To this end, HeLa cells were transfected with LEREPO4-specific siRNA molecules as described above. HeLa cells transfected with the Rhodamin-labelled non-silencing siRNA were used as a control. 48 and 72 hours after transfection, whole protein was isolated and LEREPO4 expression determined in a Western Blot. To this end, the antibodies as described above were used. In order to normalise the results, Tubulin was also detected by Western Blots.

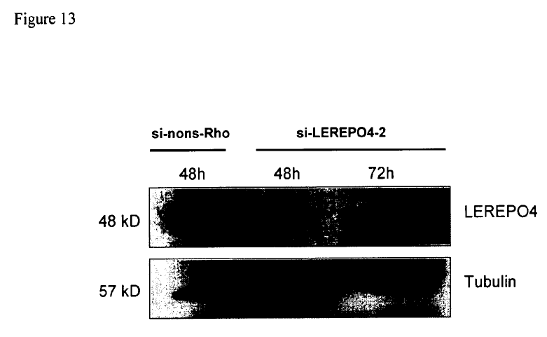

From FIG. 13 it is clear that use of LEREPO4-specific siRNA molecules indeed led to a significant reduction of expressed LEREPO4 protein.

In the case of GliPR, reduction of GliPR expression was determined by FACS analysis.

To this end, HeLa cells were transfected with plasmid coding for a GliPR-EGFP fusion protein. The GliPR-EGFP expression plasmid was co-transfected with the GliPR-specific siRNA oligonucleotides and expression of GliPR-EGFP was determined 24 hours after transfection using FACS analysis.

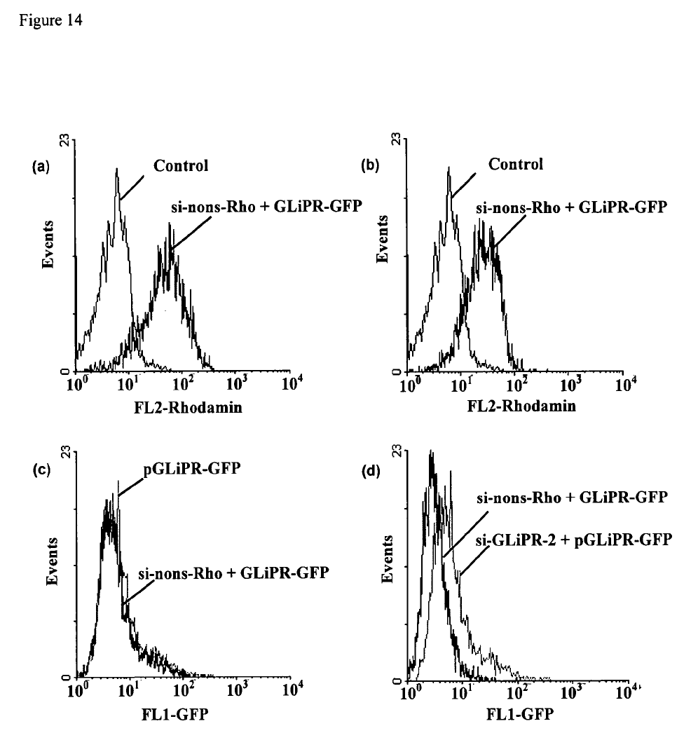

Transfection efficiency of the siRNA transfection of GliPR-specific siRNA was compared to the co-transfection of siRNA with the GliPR-EGFP expression plasmid. To that end, Rhodamin-labelled non-silencing siRNAs were transfected under standard conditions and the achieved transfection efficiency determined using FACS analysis. The same amount of Rhodamin-labelled non-silencing siRNA was co-transfected with the expression plasmid coding for the GliPR-EGFP fusion protein and transfection efficiency determined using FACS analysis. The results are shown in FIG. 14.

As can be gathered from FIGS. 14 (a) and (b), transfection efficiency was reduced for the co-transfection from 80% to approximately 60%. In contrast, transfection efficiency of the expression plasmid for GliPR-EGFP was not significantly affected by co-transfecting the Rhodamin-labelled non-silencing siRNAs.

Co-transfection of the plasmid coding for the GliPR-EGFP fusion protein with the GliPR-specific siRNA oligonucleotides led to a reduction of expression of the EGFP fusion protein from about 80% to below 50%, as can be taken from FIG. 14 (d).

This confirms that use of the above-described GliPR-specific siRNAs indeed also leads to a reduction of GliPR expression on the protein level.

Taken together, these results show that using the above-described synthetic siRNA molecule it was possible to repress expression of LEREPO4 and GliPR on the transcript and protein level.

### 3. Influence of Repression of LEREPO4 and GliPR on HIV Replication

To determine the effect of reduced expression of LEREPO4 or GliPR on HIV infectivity (as measurable by HIV replication), P4-CCR5 cells were used. These cells additionally had a β-galactosidase reporter construct for detection of productive HIV replication.

### Proliferation Test

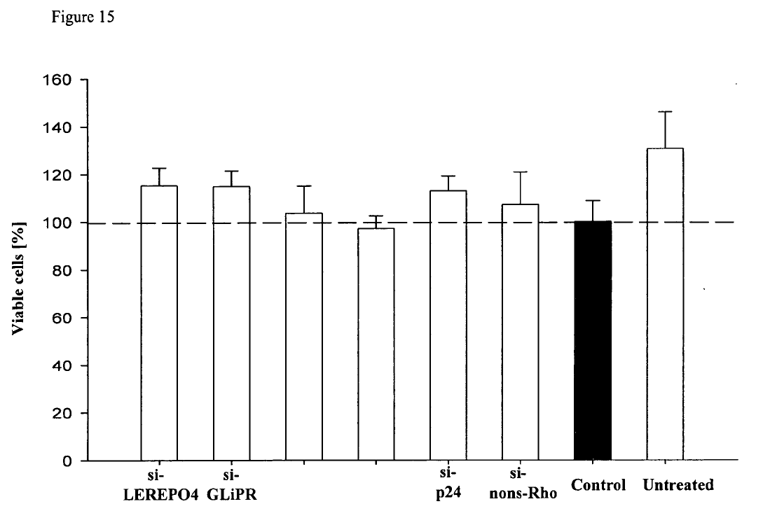

As described above, proliferation of P4-CCR5 cells after siRNA transfection was determined using WST-1 cell proliferation reagent directly before and 48 after siRNA transfection. As a control, cells which had been treated with a transfection reagent but not with siRNA were used. These latter cells were taken as 100% viable cell values. As can be taken from FIG. 15, transfection of the LEREPO4- and GliPR-specific siRNA oligonucleotides did not lead to a significantly decreased proliferation, indicating that the siRNAs do not have cytotoxic effects.

### HIV Replication in P4-CCR5 Cells After Gene Suppression

Subsequently P4-CCR5 cells were infected with the respective siRNA oligonucleotides.

As a negative control, Rhodamin-labelled non-silencing siRNAs were used. A positive control was a siRNA specific against the viral p24 gene (SEQ ID No 21). This latter antisense sequence has been shown to inhibit HIV replication in HeLa-CD4 cells (Klein S A et al. J Virol Methods. (2003), February; 107(2):169-75.) Determination of transfection efficiency showed that a transfection efficiency of at least 90% was achieved.

Infections of the cells with HIV was carried out 24 hours after transfection of the siRNAs.

To this end, the HIV strain HIV-1_{Bru} was used. Infection was carried out an MOI of 0.01 in the presence of DEAE dextrane (50 μg/ml). Samples for determining the HIV replication rate were taken on days 0, 2, 4, 6, 9 and 12 after infection. The samples were virus-containing cell culture supernatants for determination of p24 concentration via ELISA.

Additionally, total cellular RNA was isolated for determining HIV-RNA copy number using real time TaqMan PCR. It has been shown before that the intracellular HIV-RNA copy number as determined using TaqMan PCR correlates with the amount of p24 protein in the cell culture supernatant (Klein S A et al. J Virol Methods. (2003) February; 107(2): 169-75).

Further analysis of HIV replication was performed using the β-galactosidase reporter construct of the P4-CCR5 cells.

### Detection of HIV Replication Using Taqman PCR

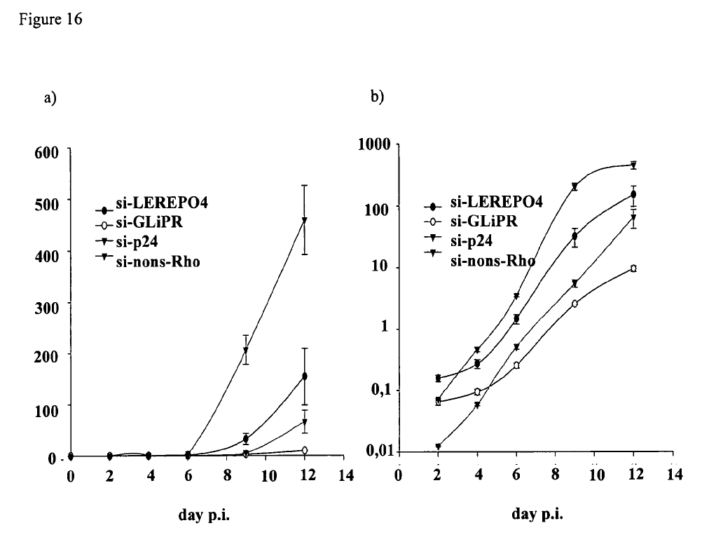

As shown in FIG. 16, repression of LEREPO4 and GliPR by way of specific siRNA oligonucleotides leads to a significant inhibition of HIV replication.

In the case of GliPR, the inhibition of HIV replication was even more pronounced than for silencing of viral p24. For LEREPO4, HIV-RNA copy number was also significantly reduced.

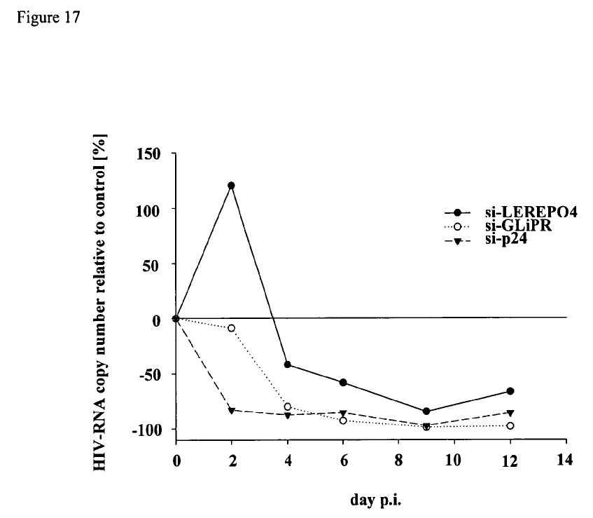

Thus, siRNA-induced repression of LEREPO4 and GliPR reduction led to a significant reduction of HIV replication. This effect was even more pronounced if the determined number of HIV-RNA copy numbers was referenced to the control, as shown in FIG. 17.

Thus, after 9 days of LEREPO4 repression, the reduction of HIV copy number was reduced by 84%. In the case of GliPR, this value was even 93%.

### Determination of HIV Replication by β-Galactosidase Activity in P4-CCR5-Cell Extracts

Results along the same lines were obtained if HIV replication was determined via β-galactosidase expression in P4-CCR5 cells.

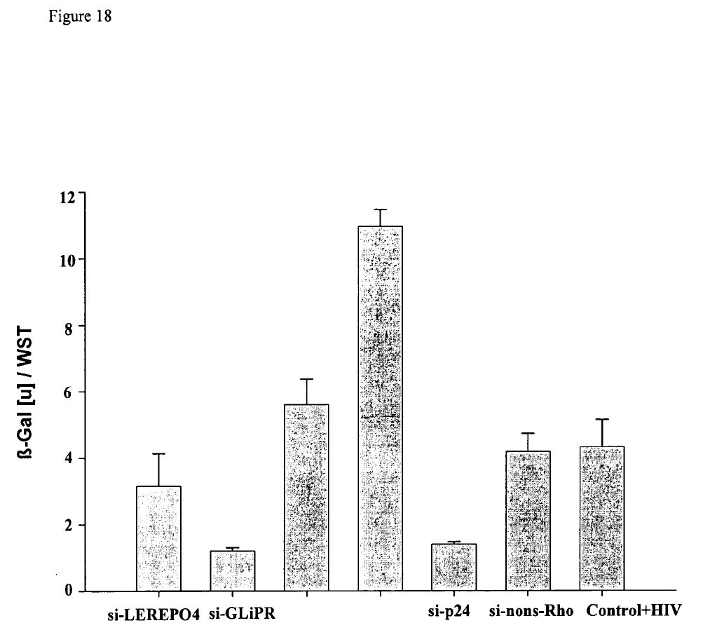

As becomes clear from FIG. 18, the β-galactosidase expression was reduced as a consequence of reduced LEREPO4 and GliPR expression.

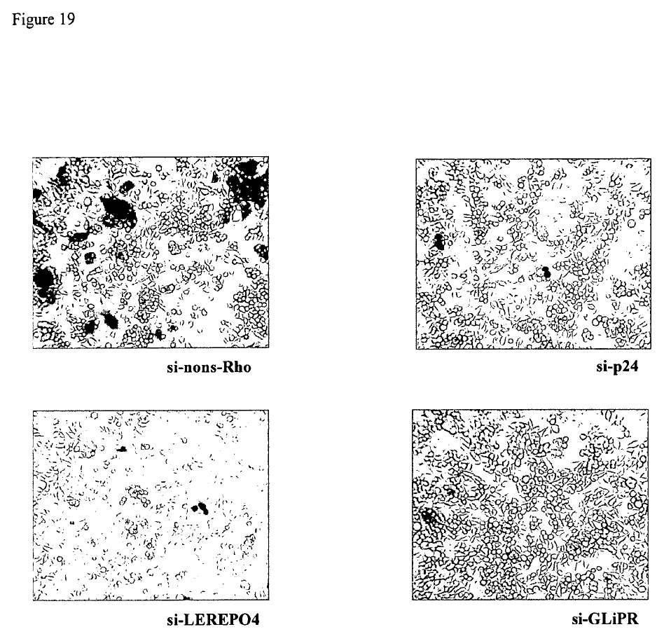

This is also confirmed by FIG. 19, which shows the staining of P4-CCR5 cells using X-Gal seven days after HIV-1 infection.

### Determination of HIV Replication by p24 ELISA

The effect of repressed LEREPO4 and GliPR expression on HIV replication was detected using a p24 ELISA.

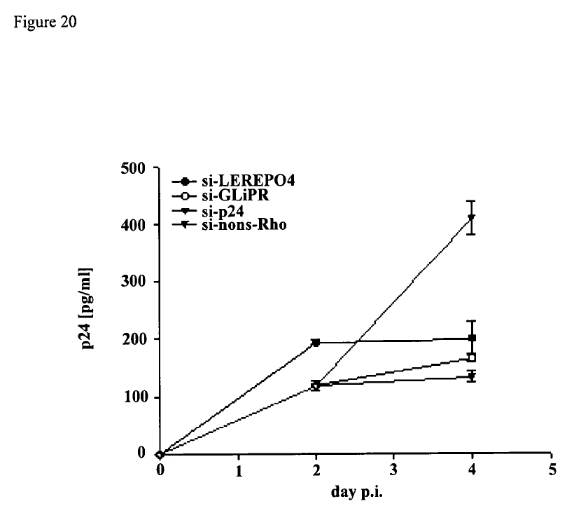

As can be taken from FIG. 20, p24 expression was reduced as a consequence of siRNA-mediated repression of LEREPO4 or GliPR.

### 4. Detection of Changes in Gene Expression by Affymetrix Microarray Analyses

Total RNA from HeLa cells transfected with siRNAs directed against LEREPO4 or GliPR (FIG. 26) was isolated using the Qiagen RNeasy Minikit, according to the suggestions of the manufacturer. The RNA quality and -concentration was determined photometrically and the RNA was subsequently used to prepare fluorescent-labelled probes for the hybridisation of Affymetrix HG-U133 Plus 2.0 Arrays.

Samples from uninfected cells served as an internal control. Analyses were performed using online resources such as NetAffx (Affymetrix), and GO Ontology. Between the ‘baseline’ (untransfected) and the ‘experimental’ (siRNA-transfected) arrays, 202 genes were differentially expressed, with 114 genes being ‘induced’ (Change of factor ≧3) and 88 genes being ‘repressed’ (Change of factor ≧−3). A selection of differentially expressed genes is given in Table 1 and -2, above.

### 5. Detection of LEREPO4-Interacting Proteins by Co-Immunoprecipitation

Immobilized LEREPO4-specific antibodies were used to isolate proteins bound to LEREPO4, using an affinity-chromatography setup (see above). As can be taken from FIG. 27, TRAF2 and LEREPO4 physically interact with each other.

6. Effect of LEREPO4 and TRAF2 and/or -6 Expression on the Activity of NF-κB

The interplay of LEREPO4 and TRAF-family proteins was further investigated by assaying the effect of LEREPO4 alone, or in combination with TRAF2 and/or -6 on the activity of the transcription factor NF-κB. As can be taken from FIG. 29 the transfection of cells with expression vectors encoding for LEREPO4, TRAF2 and TRAF6 increased the activity of NF-κB, when applied alone. The concomitant expression of all three proteins lead to an even stronger NF-κB-activation.

### 7. Subcellular Localization of LEREPO4

In an Immunofluorescence experiment, using LEREPO4-specific antibodies and a Alexa-488-conjugated sekundary antibody, LEREPO4 appears to be localized predominantly in the cytoplasm (FIG. 28).

### 8. Subcellular Localization of GliPR-EGFP

A GliPR-EGFP fusion protein in parallel with the endoplasmic reticulum-specific marker proteindisulfideisomerase (PDI, detected with a Alexa-594-coupled secondary antibody was used to investigate the subcellular localization of GliPR. From the overlay of the two fluorescence channels, it is apparent that GliPR colocalized with PDI, meaning to the ER/Golgi-compartment (FIG. 33).

### 9. Detection of Apoptosis

As can be taken form FIGS. 30-32, the expression of GliPR or fusions of GliPR with EGFP or a Strep-tag fusion elicits apotosis, characterized by the exposure of phosphatidylserine on the outer cell surface. Using the GliPR-EGFP also apoptotic DNA-degradation and characteristical morphological changes of cells were demonstrated.

In summary, the above results clearly established that down-regulation of LEREPO4 and GliPR expression being equivalent to interfering with the function of these proteins leads to decreased HIV replication in infected cells.

Thus, interfering with the function of LEREPO4 or GliPR in vivo may be a way of (i) reducing HIV replication in already infected cells and/or (ii) lowering the susceptibility of cells to infection by HIV.

Furthermore, it is apparent from the results above that LEREPO4 takes part in the signalling via TRAF2 and -6 and therefore actively participates in the control of the central immune modulator NF-κB. Also, GliPR has been characterized as an inducer of apoptosis, a phenomenon associated with a plethora of diseases and disorders.

Thus, interfering with LEREPO4 and/or GliPR-function by virtue of siRNA-mediated knockdown (see above) may be a way of reducing the occurrence of diseases relating to infection and immunity and associated disorders.

1-22. (canceled)

23. A molecule selected from the group of molecules comprising:
a) a recombinant nucleic acid molecule comprising a sequence being complementary and/or specific to the complete coding sequence or parts thereof of GLiPR (SEQ ID No.5) or functional homologues thereof;
b) a recombinant nucleic acid molecule encoding a trans-dominant proteinacious mutant of GLiPR or functional homologues thereof

24. A molecule according to claim 23a,
wherein said recombinant nucleic acid molecule of claim 23a is an shRNA, siRNA, miRNA, antisense RNA, ribozyme, antisense DNA or an aptamer to GLiPR or functional homologues thereof

25. A molecule according to claim 23a,
wherein said recombinant nucleic acid molecule of claim 23a comprises a contiguous stretch of at least 10, 11, 12, 13, 14, 15, 16, 17, 18, 19, 20, 21, 22, 23, 24, 25, 26, 27, 28, 29, 30, 50, 75, 100, 150, 200, 300 or more nucleotides.

26. A molecule according to claim 23a, wherein the degree of complementarity between said recombinant nucleic acid molecule of claim 23, a) and the complete coding sequence or parts thereof of GLiPR (SEQ ID No.5) or functional homologues thereof is at least 30%, at least 35%, at least 40%, at least 45%, at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% over a contiguous stretch of at least 10, 11, 12, 13, 14, 15, 16, 17, 18, 19, 20, 21, 22, 23, 24, 25, 26, 27, 28, 29, 30, 50, 70, 100, 150, 200, 300 or more nucleotides.

27. A molecule according to claim 23a,
wherein said recombinant nucleic acid molecule of claim 23a is selected from the group comprising SEQ ID No. 2 or parts thereof having a contiguous stretch of at least 10, 11, 12, 13, 14, 15, 16, 17, 18, 19, 20, 21, 22, 23, 24, 25, 26, 27, 28, 29, 30, 50, 70, 100, 150, 200, 300 or more nucleotides, including SEQ ID No. 17, SEQ ID No. 18 or SEQ ID No. 19.

28-33. (canceled)

34. A molecule according to claim 23b,
wherein said recombinant nucleic acid molecule of claim 23b comprises a sequence encoding one of the mutant protein sequences in FIG. 23.

35. A molecule according to claim 23b
wherein the inhibitor comprises a polypeptide sequence encoded by said recombinant nucleic acid molecule of claim 23b.

36-38. (canceled)

39. A method of inhibiting expression of GLiPR or functional homologues thereof in a mammalian cell of human or non-human origin, wherein the method comprises administering a molecule according to claim 23 to said cell.

40. (canceled)

41. A method of treating or preventing AIDS in an individual, which comprises providing to HIV infected cells of said individual, or to HIV susceptible cells of said individual a pharmaceutically active amount of a molecule or pharmaceutical composition according to claim 23.

42. (canceled)

43. A method of diagnosing AIDS and/or HIV infection in an individual and/or of determining the susceptibility of HIV strains or isolates to inhibition of GLiPR function, comprising the steps of
a) obtaining a cellular sample from an individual being potentially afflicted with AIDS and/or HIV infection;
b) determining the expression level of GLiPR in said cellular sample outside the individual's body;
c) comparing said expression level of GLiPR with expression level of GLiPR in a cellular sample obtained from an individual not afflicted with AIDS of HIV infection;
d) determining the occurrence of AIDS or HIV-1 infection by observing an increased expression level of GLiPR in b) compared to c).

44-46. (canceled)

47. A method according to claim 39, thereby attenuating, reducing or preventing infections by viruses, retroviruses, human T cell leukemia virus, Epstein-Barr virus, bacteria, Helicobacter pylori, fungi, parasites, Schistosoma haematobium, prions and/or attenuating, reducing or preventing diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies.

48. Use of a molecule or pharmaceutical composition according to claim 23 for the manufacture of a medicament in the treatment and/or prevention of infections by viruses, retroviruses, human T cell leukemia virus, Epstein-Barr virus, bacteria, Helicobacter pylori, fungi, parasites, Schistosoma haematobium, prions and/or in the treatment and/or prevention of diseases, such as disorders caused by pathological cell death rate, disorders caused by autophagy, disorders caused by pathological T cell activation, neurodegenerative disorders, Morbus Alzheimer, Morbus Parkinson, neuromuscular disorders, amyotrophic lateral sclerosis, traumata of the central nervous system, ischemic disorders of the central nervous system, degeneration of the retina, cardiovascular disorders, inflammatory disorders, chronic inflammatory diseases, rheumatoid arthritis, psoriatic arthritis, osteoarthritis, autoimmune diseases, autoimmune hepatitis, HIV-associated neoplasias, retrovirus-associated neoplasias, virus-associated neoplasias, bacteria-associated neoplasias, parasite-associated neoplasias, neoplasias, glioblastoma, drug-resistant tumors, gastrointestinal tumors, colon carcinoma, bladder carcinoma, MALT lymphoma, Burkitt lymphoma, adult T cell leukemia, adult T cell lymphoma, acute myelocytic leukemia, transmissible spongiform encephalopathies.
